(19) United States
(12) Patent Application Publication
(10) Pub. No.: US 2007/0065526 A1
(43) Pub. Date: Mar. 22, 2007

(54) Methods and compositions comprising Panax species

(76) Inventors: Robert T. Gow, Naples, FL (US); George W. Sypert, Naples, FL (US); Dan Li, Singapore (SG); Xun Yan, Nashville, TN (US)

Correspondence Address: Robert T. Gow, 1004 Collier Center Way, Suite 200, Naples, FL, 34110, US

(21) Appl. No.: 11/229,792

(22) Filed: Sep. 19, 2005

(51) Int. Cl. A61K 36/254; A61K 31/704

(52) U.S. Cl. 424/728; 514/26; 514/33

(57) ABSTRACT

The present invention comprises compositions comprising essential oils, ginsenosides, and polysaccharides. The present invention comprises methods comprising sequential solvent extraction and polymer absorbent purification to obtain fractions comprising essential oils, ginsenosides, and polysaccharides. The compositions of the present invention may be used in forms such as tablets, gel caps, or fast-dissolve tablets for use as dietary supplements or pharmaceutical compositions. The compositions of the present invention may be used for treatment of cardiovascular diseases, neurodegenerative diseases, inflammatory diseases, hepatic disorders, viral diseases, and cancer.

### FIELD OF INVENTION

The present invention relates to methods and compositions of the genus Panax (ginsengs) comprising ginsenosides, polyacetylenes, and polysaccharides and providing such compositions particularly as oral delivery formulations, and methods of use of such compositions.

### BACKGROUND OF THE INVENTION

Ginseng, the rhizome (root) of Panax ginseng (Asian ginseng, Korean ginseng) of the Araliaceae family, has been used in Oriental medicine since ancient times as a stimulant, tonic, diuretic, and digestive aid. In Europe, ginseng phytomedicines are sold over the counter and are taken to increase mental and physical performance, to provide resistance to stress and disease, and to relieve exhaustion. In 1994, European retail sales were about $50 million. Because of the continual harvest and use over thousands of years, the natural supply of P. ginseng root was exhausted long ago. Today, almost all of the P. ginseng roots are cultivated in China, Korea, and Japan.

Many congeners of ginseng are used as medicines. The root of P. quinquefolium L. (American ginseng), which originally grew wild in North America, is now cultivated for export to the Asian market where it is used medicinally for slightly different purposes than P. ginseng. P. notoginseng (also known as Sanchi ginseng) has been used as a special herb in Traditional Chinese Medicine (TCM) from ancient times to the present. Other species such as, but not limited to, P. japonicus, P. pseudo-ginseng, P. vietnamensis, Eleutherococcus senticosus (Siberian ginseng), and other species, subspecies, or varieties have also been used in Asian phytomedicine.

The constituents of the Panax rhizomes (ginseng roots) have been investigated since the beginning of the 20^{th} century. Several of the classes of compounds have been isolated and some of the individual chemical constituents have been studied for their biological effects. Some of the classes of chemical compounds that are ubiquitous to the various ginseng roots include the triterpene saponins, essential oil in which is contained the chemicals known as polyacetylenes, polysaccharides, sesquiterpenes, peptidoglycans, nitrogen-containing compounds, and others such as fatty acids, carbohydrates and phenolic compounds (2). The chemical constituents of the ginsengs that are believed to contribute to their pharmacological effects have been investigated extensively since the 1950s. The prinicipal bioactive compounds based on these investigations are the triterpene saponins, polyacetylenes, and the polysaccharides (2-7). The distribution, quantity and molecular structure of these bioactive agents vary among the Panax species and probably accounts for their different biological and medicinal activities.

All of the Panax species contain a class of triterpene saponins collectively called ginsenosides (or panoxosides). The ginsenosides contain a 4 trans-ring rigid steroid skeleton, and the individual ginsenosides differ by the number, type, and location of their sugar moieties, and the backbone structure of the triterpene or steroid moiety (3). The ginsensosides are named “ginsenosides Rₓ” wherein “x” corresponds to the sequence of R_{f} value of the spots when analyzed by thin layer chromatography. Among the known ginsenosides are R₀, Rbₐ₋₁, R_{b-2}, R_{c}, R_{d}, R_{g-3}, Rₕ₋₂, Rₑ, R_{f}, R_{g-1}, R_{g-2}, R₁, and R₂. The ginsenosides are further categorized into groups based upon the backbone structure of the steroid moiety, and include those ginsenosides based on the 20(S) protopanaxadiol backbone (collectively the the R_{b} group), the 20(S) protopanaxtriol backbone (collectively the R_{g} group), the ocotillol backbone, and the oleanane backbone. Specific ginsenosides in the R_{b} group include R_{b1}, R_{b2}, R_{c}, R_{d} and several other related compounds. Specific ginsenosides in the R_{g} group include R_{g}, Rₑ, R_{f}, R_{g2} and several other related compounds.

In view of the variable chemical constituent concentrations in the rhizomes of specific species of ginseng, the lack of selectively of the available extractions methods, and the emphasis on quantification of the ginsenoside content in current available commercial extraction processes, presently available ginseng products are suspect regarding their chemical compositions not only with respect to the ginsenoside content but also with crucial chemical constituents such as the essential oil and polysaccharides being completely absent in such products. What is needed are methods for extracting Panax and related species and Panax extraction compositions with enhanced bioactive profiles, such as, but not limited to, the triterpene saponins (e.g., ginsenosides), essential oil (e.g., polyacetylenes), and polysaccharides fractions, that can be produced with standardized and reliable amounts of these physiologically and medically beneficial bioactive Panax constituents.

### SUMMARY OF INVENTION

The present invention relates to methods for extracting and using and compositions of Panax and related species, particularly the ginsengs. In particular, the present invention comprises methods for extracting Panax compositions that have predetermined characteristics, such as, but not limited to, elevated amounts of triterpene saponins, polyacetylenes, and polysaccharides compared to the native plant material and currently available Panax extraction products. In general, such methods comprise extraction of compounds, such as triterpene saponins, polyacetylenes, and polysaccharides from extracts of native Panax plant materials or from native Panax plant material using one or more extraction steps disclosed herein.

An aspect of the invention comprises methods of selective extraction of the triterpene saponins using polymer absorbent technology different from current extraction techniques used on naturally derived material from Panax and related species.

An aspect of the invention comprises methods for extracting polyacetylene compounds and methods for extracting polysaccharide compounds from Panax and related species.

Another aspect of the invention comprises Panax and related species extraction products that have predetermined elevated concentrations of triterpene saponin, polyacetylene, and polysaccharide compounds resulting in novel profiles of these compounds in the extraction compositions unlike those found in the native plant material or in currently known extraction compositions.

The invention comprises methods of preparing extracts from Panax and related species comprising processing steps to produce compositions comprising predetermined chemical compound profiles or ratios to meet particular considerations for final products.

The compositions of the present invention may comprise pastes, resins, oils, beverages, liquid infusions or decoctions, powders, and dry flowable powders. Such products are processed for many uses, including, but not limited to, a fast dissolve tablet or other oral delivery form. The compositions taught herein can be used alone or in combination with other compounds such as other botanical materials, herbal remedies, pharmaceutical agents, food, dietary supplements, or beverages. The compositions taught herein can be used for treatment of physiological, psychological, and medical conditions.

The present invention comprises methods and compositions of formulations of oral delivery systems having the desired physiological, psychological, and medical effects that are reliable and safe. An aspect of the invention comprises extracts of Panax and related species having an elevated concentration of triterpene saponins. Another aspect of the invention comprises extracts of Panax and related species that have an elevated concentration of polyacetylene compounds. A further aspect of the invention comprises extracts of Panax and related species that have an elevated concentration of polysaccharides. Yet another aspect of the invention comprises novel profiles of the chemical constituents extracted from Panax and related species.

The compositions of the present invention are useful in providing the physiological, psychological, and medicinal effects including, but not limited to, antioxidant activity, cardiovascular protection and treatment, cytoprotection, nervous system protection, anti-neurodegenerative disease (e.g., Alzheimer's disease, Parkinson's disease, stroke), platelet aggregation inhibition, anti-cholesterol, hypoglycemia and diabetes mellitus, anti-inflammatory, immune enhancement, anti-viral (e.g., influenza), anti-pulmonary disease, hepatic protection and disease treatment, cancer prophylaxis and therapy, enhancement of male erectile function, enhancement of memory and cognition, and relief from chronic fatigue syndromes.

### BRIEF DESCRIPTION OF THE FIGURES

FIG. 1 depicts an exemplary method for the preparation of an essential oil fraction from plant feedstock.

FIG. 2 depicts an exemplary method for the preparation of ginsenoside fractions.

FIG. 3 depicts an exemplary method for the preparation of a purified ginsenoside fraction using a polymer adsorbent.

FIG. 4 depicts an exemplary method for the preparation of polysaccharide fractions.

FIG. 5 shows an HPLC chromatogram of an essential oil fraction obtained from P. notoginseng following supercritical fluid extraction.

FIG. 6 shows an HPLC chromatogram of an essential oil fraction obtained from P. quinquefolius following supercritical fluid extraction.

FIG. 7 shows an HPLC chromatogram of an essential oil fraction obtained from white ginseng (P. ginseng) following supercritical fluid extraction.

FIG. 8a shows an HPLC chromatogram of an essential oil fraction obtained from red ginseng (P. ginseng) following supercritical fluid extraction.

FIG. 8b shows an expanded portion of the HPLC chromatogram shown FIG. 8a, wherein the expanded portion corresponds to elution time of about 8.0 minutes to 12.5 minutes.

FIG. 9 shows an HPLC chromatogram of a ginsenoside fraction obtained from P. notoginseng following purification using a polymer adsorbent resin.

FIG. 10 shows an HPLC chromatogram of a ginsenoside fraction obtained from P. quinquefolius following purification using a polymer adsorbent resin.

FIG. 11 shows an HPLC chromatogram of a ginsenoside fraction obtained from white ginseng (P. ginseng) following purification using a polymer adsorbent resin.

FIG. 12 shows an HPLC chromatogram of a ginsenoside fraction obtained from red ginseng (P. ginseng) following purification using a polymer adsorbent resin.

FIG. 13 shows a GC chromatogram of an essential oil fraction obtained from P. notoginseng following supercritical fluid extraction.

FIG. 14 shows a GC chromatogram of an essential oil fraction obtained from P. quinquefolius following supercritical fluid extraction.

FIG. 15 shows a GC chromatogram of an essential oil fraction obtained from white ginseng (P. ginseng) following supercritical fluid extraction.

FIG. 16 shows a GC chromatogram of an essential oil fraction obtained from red ginseng (P. ginseng) following supercritical fluid extraction.

FIG. 17 shows an MS spectrum of a fraction eluting at 29.975 minutes in the GC chromatogram for an essential oil fraction obtained from P. notoginseng.

FIG. 18 shows an MS spectrum of a fraction eluting at 31.173 minutes in the GC chromatogram for an essential oil fraction obtained from P. notoginseng.

FIG. 19 shows an MS spectrum of a fraction eluting at 32.775 minutes in the GC chromatogram for an essential oil fraction obtained from P. notoginseng.

FIG. 20 shows an MS spectrum of a fraction eluting at 31.338 minutes in the GC chromatogram for an essential oil fraction obtained from P. quinquefolius.

FIG. 21 shows an MS spectrum of a fraction eluting at 31.852 minutes in the GC chromatogram for an essential oil fraction obtained from P. quinquefolius.

FIG. 22 shows an MS spectrum of a fraction eluting at 33.185 minutes in the GC chromatogram for an essential oil fraction obtained from P. quinquefolius.

FIG. 23 shows an MS spectrum of a fraction eluting at 29.931 minutes in the GC chromatogram for an essential oil fraction obtained from white ginseng (P. ginseng).

FIG. 24 shows an MS spectrum of a fraction eluting at 31.276 minutes in the GC chromatogram for an essential oil fraction obtained from white ginseng (P. ginseng).

FIG. 25 shows an MS spectrum of a fraction eluting at 32.903 minutes in the GC chromatogram for an essential oil fraction obtained from white ginseng (P. ginseng).

FIG. 26 shows an MS spectrum of a fraction eluting at 45.377 minutes in the GC chromatogram for an essential oil fraction obtained from white ginseng (P. ginseng).

FIG. 27 shows an MS spectrum of a fraction eluting at 7.964 minutes in the GC chromatogram for an essential oil fraction obtained from red ginseng (P. ginseng).

FIG. 28 shows an MS spectrum of a fraction eluting at 31.861 minutes in the GC chromatogram for an essential oil fraction obtained from red ginseng (P. ginseng).

FIG. 29 shows an MS spectrum of a fraction eluting at 32.888 minutes in the GC chromatogram for an essential oil fraction obtained from red ginseng (P. ginseng).

FIG. 30 shows an MS spectrum of a fraction eluting at 33.157 minutes in the GC chromatogram for an essential oil fraction obtained from red ginseng (P. ginseng).

FIG. 31 shows an MS spectrum of a fraction eluting at 35.138 minutes in the GC chromatogram for an essential oil fraction obtained from red ginseng (P. ginseng).

### DESCRIPTION OF INVENTION

The present invention comprises methods and compositions comprising Panax. The present invention comprises compositions of extracts obtained from Panax using the methods of extraction of the present invention. The compositions of the present invention comprise extract fractions from Panax comprising essential oil compositions, ginsenoside compositions, and polysaccharide compositions. The essential oil compositions comprise components detected by gas chromatography and high pressure liquid chromatography as described herein. The ginsenoside compositions comprise components detected by high pressure liquid chromatography as described herein.

In one aspect, the present invention comprises Panax compositions wherein the Panax compositions comprise elevated concentrations of triterpene saponin, polyacetylene, and polysaccharide compounds. In another aspect, the present invention comprises Panax compositions resulting in novel profiles or concentrations of these compounds in the extraction compositions unlike those found in the native plant material or in currently known extraction compositions.

The compositions of the present invention may comprise pastes, resins, oils, beverages, liquid infusions or decoctions, powders, and dry flowable powders. Such products are processed for many different uses, including, but not limited to, a fast dissolve tablet or other oral delivery form. The compositions taught herein can be used alone or in combination with other compounds such as other botanical materials, herbal remedies, pharmaceutical agents, food, dietary supplements, or beverages. The compositions taught herein can be used for treatment of physiological, psychological, and medical conditions.

In one aspect, the present invention comprises compositions comprising extracts of Panax having an elevated concentration of triterpene saponins. In another aspect, the present invention comprises compositions comprising extracts of Panax that have an elevated concentration of polyacetylene compounds. In a further aspect, the present invention comprises compositions comprising extracts of Panax that have an elevated concentration of polysaccharides.

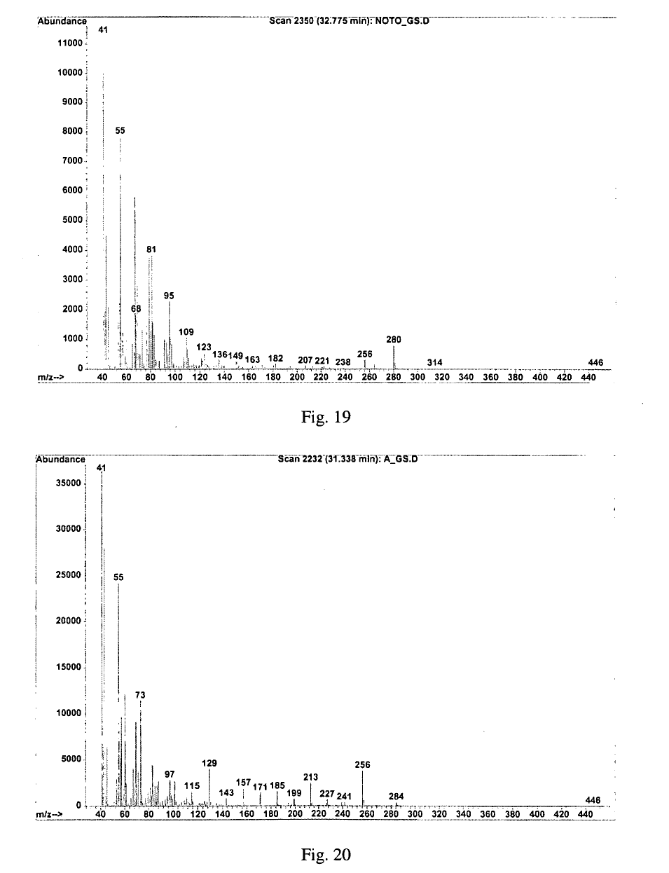

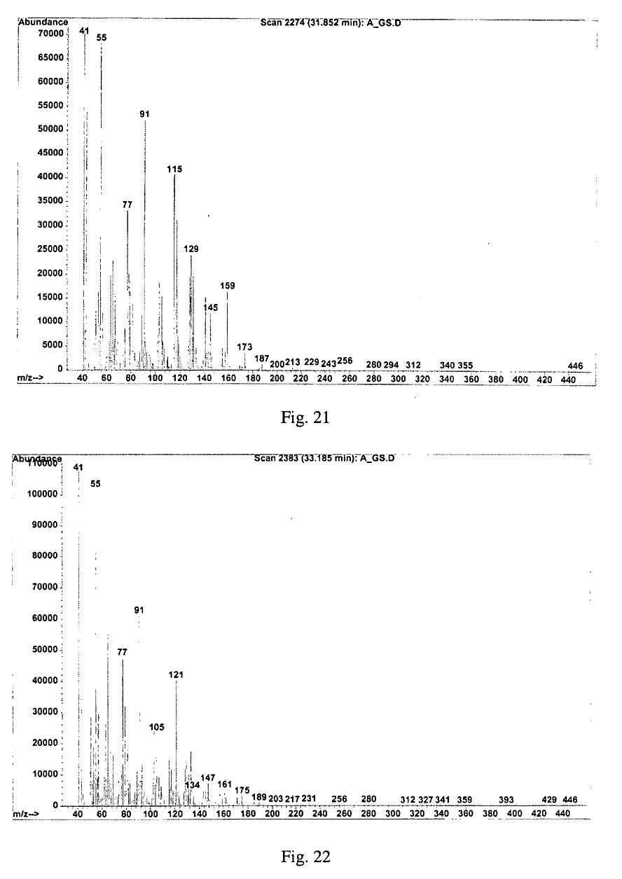

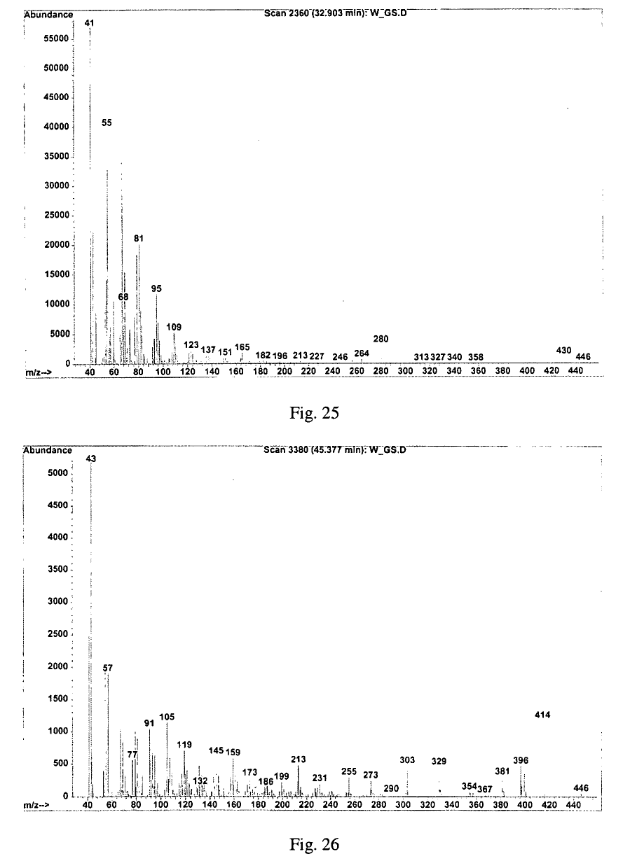

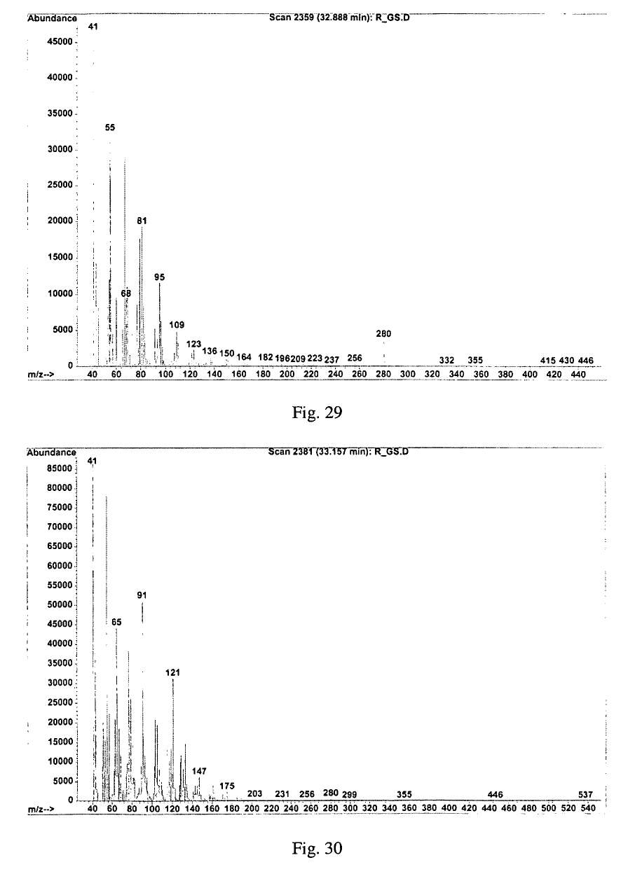

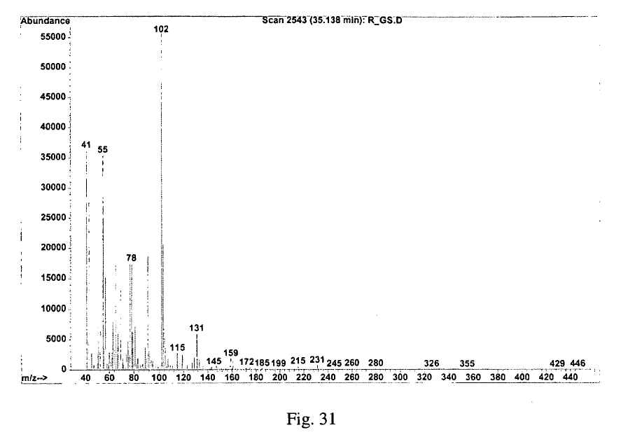

The present invention comprises compositions comprising novel profiles of the chemical constituents extracted from Panax, wherein the profile of chemical constituents may be characterized by a specific HPLC chromatogram. The present invention also comprises compositions comprising novel profiles of the chemical constituents extracted from Panax, wherein the profile of chemical constituents may be characterized by a specific MS spectrogram, or other detection system. For example, compositions of the present invention comprise compositions comprising one or compounds and combinations of compounds shown in FIGS. 13 to 31.

The present invention comprises compositions comprising formulations suitable for oral delivery of the compositions of the present invention, wherein the formulation composition provides desired physiological, psychological, and medical effects that are reliable and safe.

The present invention comprises methods for preparing the Panax compositions of the present, wherein the Panax compositions that have predetermined characteristics, such as, but not limited to, elevated amounts of triterpene saponins, polyacetylenes, and polysaccharides compared to the native plant material and currently available Panax extraction products. In general, such methods comprise extraction of compounds, such as triterpene saponins, polyacetylenes, and polysaccharides from extracts of native Panax plant materials or from native Panax plant material using one or more extraction steps disclosed herein.

The present invention comprises methods of extraction of Panax comprising supercritical fluid extraction, solvent extraction and polymer adsorbent extraction. In another aspect, the present invention comprises methods for extracting polyacetylene compounds and methods for extracting polysaccharide compounds from Panax and related species. The invention comprises methods of preparing extracts from Panax and related species comprising processing steps to produce compositions comprising predetermined chemical compound profiles or ratios to meet particular considerations for final products.

The present invention further comprises methods of preparing formulations suitable for oral delivery of the compositions of the present invention, wherein the formulations provide desired physiological, psychological, and medical effects that are reliable and safe.

As used herein, the term “essential oil fraction” comprises compounds that are volatile, water-insoluble, and extractable using non-polar solvents. As used herein, the essential oil fraction further comprises polyacetylenes obtained from Panax and related species. The essential oil fraction may further comprise one or more compounds from sesquiterpenes, azulene, patchoulene, sesquiterpene alcohols, panasinsanol A, panasinsanol B, methoxypyrazine, β-elemene, diene panaxynols, or alkylpyrazines. The polyacetylenes of the essential oil fraction may further comprise one or more compounds from pananaxynol, panaxydiol, panaxytriol, acetylpanaxydol, panaxydolchlorohydrin, panaxyne, ginsenoyne A, ginsenoyne B, ginsenoyne C, ginsenoyne D, ginsenoyne E, ginsenoyne F, ginsenoyne G, ginsenoyne H, ginsenoyne I, ginsenoyne J, ginsenoyne K, panaxacol, panaxydol, falcarinol or falcarintriol.

As used herein, the term “feedstock” refers to raw plant material, comprising whole plants alone, or in combination with one or more constituent parts of a plant comprising leaves, rhizomes, roots, including, but not limited to, main roots, tail roots, and fiber roots, stems, leaves, seeds, and flowers, wherein the plant or constituent parts may comprise material that is raw, dried, steamed, heated or otherwise subjected to physical processing to facilitate processing, which may further comprise material that is intact, chopped, diced, milled or otherwise processed to affected the size and physical integrity of the plant material.

As used herein, the term “fraction” means a composition comprising a specific group of compounds characterized by certain physical, chemical properties, or physical and chemical properties.

As used herein, the term “ginseng constituents” shall mean compounds found in each of the individual Panax and related species and shall include all such chemicals compounds identified above as well as other compounds found in each Panax and related species, including but not limited to essential oils, polyacetylenes, ginsenosides, and polysaccharides.

As used herein, the term “ginsenoside fraction” comprises triterpene saponins obtained from Panax and related species, further comprising compounds based on the protopanaxadiol backbone, the protopanaxtriol backbone, the ocotillol backbone, or the oleanane backbone, and related compounds.

As used herein, the term “increased” or “elevated” amount of a fraction, including, but not limited to, fractions such as the triterpene saponin, polyacetylene, and polysaccharide fractions, means that the weight percent of the fraction, either in toto or a single constituent of the fraction, in a mixture or sample is increased compared to the weight percent of the fraction in the native plant or plant tissue.

As used herein, the term “one or more compounds” means that at least one compound, such as panaxytriol (an essential oil polyacetylene), R_{g1}, (a ginsenoside triterpene saponin), or ginsenan PA (a water soluble ginseng polysaccharide) is intended, or that more than one compound, for example, panaxytriol and R_{g1} is intended. As known in the art, the term “compound” does not mean a single molecule, but multiples or moles of molecules. As known in the art, the term “compound” means a specific chemical entity possessing distinct chemical and physical properties, whereas “compounds” refers to one or more chemical constituents.

As used herein, the term “Panax” comprises the genus Panax and related species, including, but not limited to, Eleutherococcus senticosus. Further, as used herein, Panax refers to the plant or plant material derived from the plant Araliaceae family, wherein the species includes but is not limited to, P. ginseng, P. quinquefolius, P notoginseng, P. pseudoginseng, P. japonicum, P. vietnamensis, and E. senticosus. The term also includes all clones, cultivars, variants, and sports of Panax and related species. The term “Panax” may also be used herein interchangeably with “ginseng” and means these plants, clones, cultivars, variants, and sports.

As used herein, the term “polysaccharide fraction” comprises compounds obtained or derived from Panax and related species. The polysaccharide extract fraction of the chemical constituents of the Panax species has been defined in the scientific literature as the “ethanol insoluble-water soluble extraction fraction” (26, 31, 63, 72-74). The polysaccharide fraction may comprise one or more compounds from ginsan and panaxans A through U, including, but not limited to, the neutral polysaccharides panaxans A through E and the acidic polysaccharides panaxan Q through U. The polysaccharide fraction may further comprise saccharide polymers and oligomers comprising monomer units from glucose, arabinose, galactose, rhamnose, xylose, or uronic acid.

As used herein, the term “rhizome” refers to the constituent part of Panax and related species comprising a horizontal root stem, which may be in part or in whole, be underground, further comprising shoots above and roots below, including, but not limited to, main roots, tail roots, and fiber roots.

Overviews of the pharmacological effects of P. ginseng extracts and preparations (ginsenoside, polyacetelene and polysaccharide fractions) have been presented by many authors (2-8). The preparation and definition of products derived from ginseng is specified in various European pharmacopoeias. The Swiss pharmacopoeia, Pharmacopoea Helvetica (Commission Suisse de Pharmcopée), requires a total ginsenoside content, calculated as relative to the abundance of ginsenoside R_{g1}, of not less than 2.0%. According to the German pharmacopoeia (Herbal Medicine—Expanded Commission E Monographs, Blumenthal, M. et al., Integrative Medicine Communications, 2000, pp. 170-177), the total ginsenoside content should be not less than 1.5% using a spectrophotometric method of quantification. In contrast, the 4^{th} Edition of the European Pharmacopoeia (European Pharmacopoeia Commission, European Directorate for the Quality of Medicines—Council of Europe, 2001) requires the content of ginsenosides R_{g1} and R_{b1} to be not less than 0.3%, measured using High Performance Liquid Chromatography (HPLC) methods. However, consumers in the U.S. who purchase a ginseng product should carefully consider the source and product. No federal agency enforces quality control over the ingredients of many products. In study of 54 so-called ginseng products, it was found that 25% contained no ginsenosides at all, and 60% contained only trace amounts (8-10). These ginseng extracts are further compromised by the fact that the ginseng feedstocks (raw natural plant material) contain varying amounts of the bioactive chemicals depending on many variables during the growth cycle such as soil and air quality, rainfall or light exposure.

The principal physiological effects upon ingestion of ginseng that is documented in the older literature (2) include the following: general tonic; stimulation of immunological function; beneficial effects on the cardiovascular system including a lowering of blood pressure; reductions in serum total cholesterol, low-density lipoprotein cholesterol and triglyceride levels and increases in serum high-density lipoprotein cholesterol levels; stimulation of alcohol dehydrogenase and oxidation of alcohol in the liver; lowering of blood sugar levels; stimulation of the pituitary-adrenocortical system, anti-aging, and inhibition of tumor growth. Interestingly, Rg1 is claimed to stimulate the central nervous system and enhances protein, DNA, and RNA synthesis whereas Rb1 has tranquilizing effects and improves memory which may again account for the different biological effects associated with the different species of Panax (6).

Recent experimental and clinical studies have demonstrated the following bioactive properties of the various chemicals and chemical fractions of the Panax species: powerful antioxidant activity (Rg1, Rb1, extract) (11-17); cardiovascular protection (Rg1, Rb1, extract) (11-21); immunological enhancement and anti-viral, anti-influenza (Rg1, polysaccharides, extract) (22-34); cytoprotection (polysaccharides, extract) (17, 20, 35); neuroprotection and anti-dementia (ginsenosides, Rb1, Rg2, Rg3, extract) (36-40); platelet aggregation inhibition (ginsenosides, Ro, Rg1, Rg2, polyacetylenes, extract) (41-44); calcium channel inhibition (Rf) (45): anti-cancer (Rg3, Rh2, polyacetylenes, polysaccharides) (46-52); anti-inflammatory (polyacetylenes, extract) (53, 54); anti-cholesterol (extract) (55, 56); hypoglycemic and anti-diabetes (polysaccharide, extract) (57-60); pulmonary disease protection and therapy (extract) (34, 61); hepatic protection and disease treatment (extract) (62, 63); enhancement of erectile capacity (extract) (64-67); enhanced memory and cognition (Rb1, Rg1, polyacetylenes, extract) (37, 38, 68, 69); and chronic fatigue (extract) (70, 71).

Each member of the Panax species appears to differ in the amounts of individual chemicals present in the native plant material and these amounts can be analytically determined. The individual Panax species appear to have differing distributions of the major bioactive fractions, the essential oils, the ginsenosides, and the polysaccharides, which may contribute to the different physiological, psychological and medical effects that are attributed to the different species. The Panax species extraction products available prior to the current invention were merely reflections of the variability of the native plant materials, and for example, had widely fluctuating amounts of only ginsenosides, if any were present. In contrast, the present invention comprises compositions of isolated and purified fractions of essential oils, ginsenosides and polysaccharides from one or more Panax species. These individual fraction compositions can be combined in specific ratios (profiles) to provide beneficial combination compositions and can provide extract products that are not found in currently known extract products. For example, an essential oil fraction from one species may be combined with a ginsenosides fraction from the same or different species, and that combination composition may or may not be combined with a polysaccharide fraction from a same or different species of Panax.

Tables 1 through 4 list the principal beneficial bioactive chemical constituent fractions found in the four major Panax species rhizome feedstocks used to produce ginseng products.

TABLE 1
Chemical Constituent Fractions of Panax notoginseng Rhizome*

| Constituents | Essential Oil | Ginsenoside | Polysaccharide |
|---|---|---|---|
| (Source) | Fraction | Fraction | Fraction |
| Literature | 0.045-0.056 | 8.6-9.1 | — |
| ARS-PED** | — | 8.7 | — |
| HS Lab*** | 0.3-0.5 | 12.0 | 47.0 |

*% mass dry weight = (mass weight of fraction/mass weight of feedstock).
**USDA Agricultural Research Service
***HerbalScience Laboratory.

TABLE 2
Chemical Constituent Fractions of American
Ginseng (Panax quinquefolius L. Rhizome)

| Constituents | Essential Oil | Ginsenoside | Polysaccharide |
|---|---|---|---|
| (Source) | Fraction | Fraction | Fraction |
| Literature | — | 5-6 | — |
| ARS-PED | — | 2.4 | 4-20 |
| HS Lab | 0.1-0.3 | 2.3 | 18 |

TABLE 3
Chemical Constituent Fractions of White
Ginseng (Panax ginseng C. Rhizome)

| Constituents | Essential Oil | Ginsenoside | Polysaccharide |
|---|---|---|---|
| (Source) | Fraction | Fraction | Fraction |
| Literature | — | 2.5-3 | — |
| ARS-PED | — | 4.7 | 22 |
| HS Lab | 0.5 | 3.2 | 17.4 |

TABLE 4
Chemical Constituent Fractions of Red
Ginseng (Panax ginseng C. Rhizome)

| Constituents | Essential Oil | Ginsenoside | Polysaccharide |
|---|---|---|---|
| (Source) | Fraction | Fraction | Fraction |
| Literature | — | 1.5-2.8 | — |
| ARS-PED | — | — | — |
| HS Lab | 0.5 | 1.84 | 26.13 |

Compositions of the present invention comprise extracts of Panax plant material and related species in forms such as a paste, powder, oils, liquids, suspensions, solutions, or other forms, comprising, one or more fractions comprising polyacetylenes or essential oils, ginsenosides, or polysaccharides, to be used as dietary supplements, nutraceuticals, or pharmaceutical preparations and such compositions may be used to prevent or treat various human ailments. The extracts can be processed to produce such consumable items, for example, by mixing with them into a food product, in a capsule or tablet, or providing the paste itself for use as a dietary supplement, with sweeteners or flavors added as appropriate. Accordingly, such preparations may include, but not limited to, compositions of Panax and related species extract compositions for oral delivery in the form of tablets, capsules, lozenges, liquids, and emulsions. Other aspects of the compositions of the present invention comprise Panax species extract compositions in the form of a rapid dissolve tablet.

The present invention comprises compositions comprising one or more chemical constituent fractions found in each of the Panax and related species. The invention also comprises ingestible products that comprise the compositions comprising the Panax and related species extraction compositions taught herein. For example, the present invention comprises compositions comprising a rapid dissolve tablet, comprising a Panax or related species extract composition wherein at least one of an essential oil fraction, a ginsenoside fraction, or a polysaccharide fraction has been substantially increased in weight percent amount in relation to the weight percent amount of that found in the native plant material or to that currently found in known Panax species extract compositions. The present invention comprises compositions and methods for making and using Panax and related species compositions, wherein the compositions comprise oral delivery dosage formulations, comprising the compositions taught herein. Such compositions include compositions that have predetermined amounts of at least one of the essential oil, ginsenoside, or polysaccharide fractions. Embodiments comprise compositions of Panax and related species having at least one of an essential oil, ginsenoside, or polysaccharide concentration that is in an amount greater than that found in the native Panax and related species plant material or currently available Panax species extract products. Embodiments also comprise compositions wherein one or more of the fractions, including essential oils, ginsenosides, or polysaccharides, are found in a concentration that is greater than that found in native Panax species plant material. Embodiments also comprise compositions wherein one or more of the fractions, including essential oils, ginsenosides, or polysaccharides, are found in a concentration that is less than that found in native Panax species. Known amounts of four Panax species are shown here in Tables 1-4. For example, compositions of the present invention comprise compositions where the concentration of essential oils is from 0.001 to 200 times the concentration of native Panax species, and/or compositions where the concentration of ginsenosides is from 0.001 to 100 times the concentration of native Panax species, and/or compositions where the concentration of polysaccharides is from 0.01 to 6 times the concentration of native Panax species. Compositions of the present invention comprise compositions where the concentration of essential oils is from 0.1 to 50 times the concentration of native Panax species, and/or compositions where the concentration of ginsenosides is from 0.1 to 50 times the concentration of native Panax species, and/or compositions where the concentration of polysaccharides is from 0.01 to 6 times the concentration of native Panax species.

Methods of the present invention comprise providing novel Panax compositions for treatment and prevention of human disorders. For example, a novel Panax species composition for antioxidant activity and cardiovascular protection may have an increased ginsenoside fraction composition concentration, a reduced essential oil fraction composition concentration, and an increased polysaccharide fraction composition concentration, by % weight, than that found in the Panax species native plant material or conventional known extraction products. A novel Panax species composition for immune enhancement may have an increased ginsenoside fraction composition and a polysaccharide fraction composition, and a reduced essential oil fraction composition concentration, by % weight, than that found in the native Panax species plant material or conventional known extraction products. Another example of a novel Panax speicies composition, for treatment of Alzheimers disease, dementia, and enhancement of memory and cognition, comprises a composition having an increased essential oil fraction composition concentration and a ginsenoside fraction composition, and a reduced polysaccharide fraction composition than that found in native Panax species plant material or known conventional extraction products. Additional embodiments comprise compositions comprising altered profiles (ratio distribution) of the chemical constituents of the Panax species in relation to that found in the native plant material or to currently available Panax species extract products. For example, the essential oil fraction may be increased or decreased in relation to the ginsenoside and/or polysaccharide concentrations. Similarly, the ginsenosides or polysaccharides may be increased or decreased in relation to the other extract constituent fractions to permit novel constituent chemical profile compositions for specific biological effects. By combining the isolated and purified fractions of one or more of essential oils, ginsenosides and/or polysaccharides, compositions may be made that provide novel combinations of essential oils such as those taught in any one of the compositions or compounds of represented in FIGS. 5-8, and/or with any one of the compositions or compounds of represented in FIGS. 9-12, and/or with any one of the compositions or compounds of represented in FIGS. 13-31.

The starting material for extraction is plant material from one or more Panax species, though P. notoginseng, P. ginseng, which may either be in the form commonly known as “white ginseng” or the form commonly known as “red ginseng”, or P. quinquefolius are the preferred starting materials. As used herein, “white ginseng” comprises the material derived from P. ginseng which has dried in open air or in a dryer following harvesting such that the color does not become red to brown in color. As used herein, “red ginseng” comprises material derived from P. ginseng which has been heated, typically by steaming, and then dried in a manner such that the material becomes red to brown in color. The material may be the aerial portion of the plant, which includes the leaves, stems, or other plant parts, though the rhizome is the preferred starting material.

The Panax species plant material may undergo pre-extraction steps to render the material into any particular form, and any form that is useful for extraction is contemplated by the present invention. Such pre-extraction steps include, but are not limited to, that wherein the material is chopped, minced, shredded, ground, pulverized, cut, or torn, and the starting material, prior to pre-extraction steps, is dried or fresh plant material. A preferred pre-extraction step comprises grinding and/or pulverizing the Panax species rhizome material into a fine powder. The starting material or material after the pre-extraction steps can be dried or have moisture added to it. Once the Panax species plant material is in a form for extraction, methods of extraction are contemplated by the present invention.

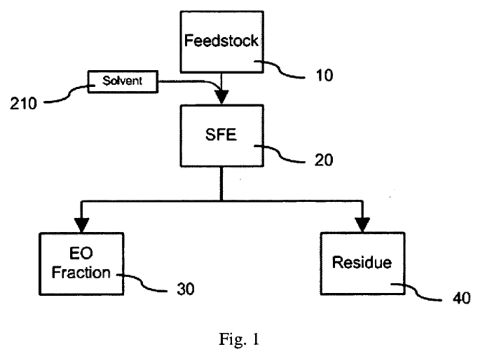

Methods of extraction of the present invention comprise processes disclosed herein. In general, methods of the present invention comprise, in part, methods wherein Panax species plant material is extracted using supercritical carbon dioxide (SCCO₂) that is followed by one or more solvent extraction steps, such as, but not limited to, water, hydroalcoholic, and polymer absorbent extraction processes. Additional other methods contemplated for the present invention comprise extraction of Panax species plant material using other organic solvents, refrigerant chemicals, compressible gases, sonification, pressure liquid extraction, high speed counter current chromatography, molecular imprinted polymers, and other known extraction methods. Such techniques are known to those skilled in the art. In one aspect, compositions of the present invention may be prepared by a method comprising the steps depicted schematically in FIGS. 1, 2, 3, and 4.

Supercritical Fluid Extraction of Panax

Due to the hydrophobic nature of the essential oil, non-polar solvents, including, but not limited to SCCO₂, hexane, petroleum ether, and ethyl acetate may be used for this extraction process.

A generalized description of the extraction of the essential oil fraction from the rhizome of the Panax species using SSCO₂ is diagramed in FIG. 1. The feedstock 10 is ground Panax species rhizome (8 to 20 mesh). The solvent 210 is pure CO₂. The feedstock is loaded into a basket that is placed inside a supercritical fluid extraction (SFE) vessel 20. After purge and leak testing, the process comprises liquefied CO2 flowing from a storage vessel through a cooler to the CO₂ pump. Then the CO₂ is compressed to the desired pressure flows through the feedstock in the extraction vessel where the pressure and temperature are maintained at the desired levels. The pressures for extraction range from about 100 to about 800 bar, from about 200 to about 600, from about 300 to about 400 bar, and the temperatures range from about 50° C. to about 120° C., and from about 60° C. to about 100° C., and from about 80° C. to about 90° C. The time for extraction range from about 30 minutes to about 2.5 hours, from about 1 hour to about 2 hours, to about 1.5 hours. The solvent to feed ratio is typically 17-18 to 1 for each of the SCCO₂ extractions. The extracted and purified essential oil fraction is then collected in a collector vessel 30, saved and stored in a dark refrigerator to 5° C. The CO₂ is recycled. The Panax species feedstock residue 40 is also collected from the extraction vessel, saved and used for further extractions of the chemical constituents in the Panax species rhizome. Typically, the total yield of essential oil fractions from Panax species varies from 0.2% to 0.0.5% mass dry weight of the original feedstock having an essential oil fraction chemical constituent composition of essentially 100% purity. The purity is measured using HPLC and GC-MS analysis (see Examples 1-4 and FIGS. 5-8b and 17-31).

Ginsenoside Extraction Process

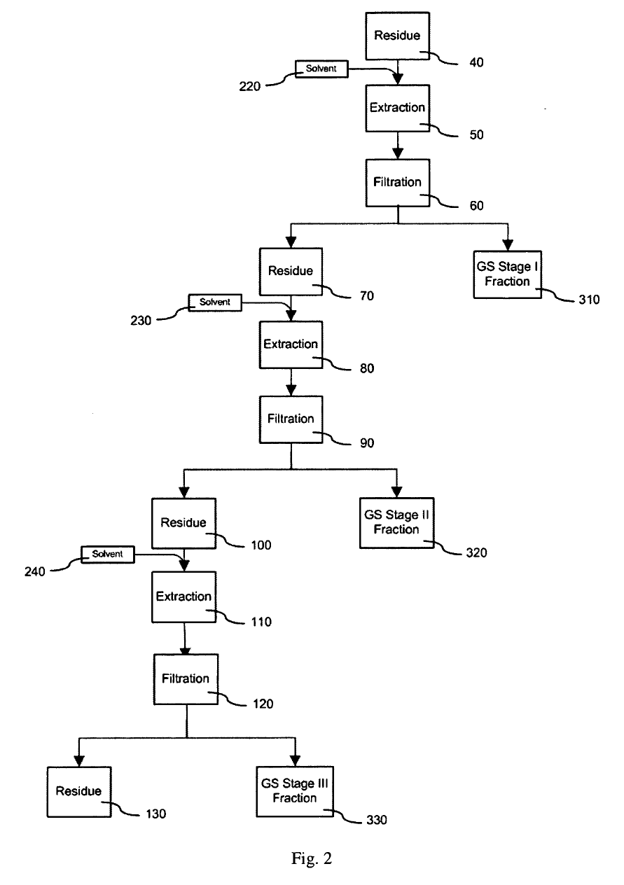

In one aspect, the present invention comprises extraction and concentration of the ginsenosides or triterpene saponins. A generalized description of this extraction step is diagrammed in FIG. 2. This ginsenoside extraction process is a 3 stage solvent leaching process. The feedstock for this ginsenoside process is the residue 40 or 45 following extraction of the essential oil fraction. The extraction solvent 220, 230, 240 is typically 63% ethanol in water. In this method, the Panax species residue and the extraction solvent are loaded into an extraction vessel heated. 50 It may be heated to less than 100° C., to about 90° C., 80° C., or to about 50-60° C. The extraction is carried out for about 1-4 hours, for about 3 hours, for about 2 hours. The resultant fluid extract is filtered 60. The filtrate is collected as product 310, measured for volume and solid content dry mass weight after evaporation of the solvent. The extraction residue 70 material retained by the filter. The extraction may be repeated as many times as is necessary or desired. It may be repeated 2 or more times, 3 or more times, four or more times, etc. For example, FIG. 2 shows a three stage process, where the second stage 80 and the third stage 110 uses the same methods and conditions. Examples are seen in Examples 5 through 8 and Tables 5 through Table 8, respectively.

Although over 30 individual ginsenoside compounds have been detected and characterized in the genus Panax, the majority of these exist in only trace amounts. The seven ginsenosides (Rg1, Re, Rf, Rb1, Rc, Rb2, Rd) that account for greater than 95% of the total ginsenoside content of the Panax species are measured throughout the extraction processes using HPLC analysis (see FIGS. 9 to 12) permitting computation of the % dry weight of the individual and total ginsenoside content in the extract products (Tables 5 to 20). The HPLC analysis was accomplished using a Shimadzu SE0405003 HPLC with analytical reference standards obtained from Chromadex, KIT-00007226-005 (ginsenosides standard kit) (see Examples 22-25).

As shown in Tables 5 through 12, a two stage solvent leaching process can give a total ginsenoside yield of at least 96% for each of the four Panax species used as a feedstocks studied while simultaneously increasing the purity of the ginsenosides in the ginsenoside extract fraction at least 4-fold. In the particular case of P. notogensing, the ginsenoside concentration of the feedstock was 12.26% mass weight and the ginsenoside concentration in the ginsenoside fraction from recombination of the stage I and stage II extractions was 50.5% by dry weight, thus a 5 fold increase in the ginsenoside concentration. For P. quinquefolius, the ginsenoside concentration in the feedstock was 2.32% mass weight and the concentration in the two stage leaching ginsenoside extract fraction was 15.2%, thus a 6 fold increase in ginsenoside concentration. For White ginseng (P. ginseng), the ginsenoside concentration in the feedstock was 3.19% and the concentration in the two stage leaching ginsenoside extract fraction was 8.9%, thus a 3 fold increase in ginsenoside concentration. Finally, for Red ginseng (P. ginseng), the ginsenoside concentration in the feedstock was 1.84% and the concentration in the two stage leaching ginsenoside extract fraction was 4.6%, thus a 2.6 fold increase in total ginsenoside concentration. Furthermore, the concentration distribution or profile of the individual ginsenosides within the extract fraction obtained from the two stage solvent leaching method is well preserved relative to the ginsenoside profile found in each of the original feedstock material. In conclusion, a two stage solvent leaching process is a very efficient and cost effective method for the extraction of the highly purified ginsenoside fraction from the plant material of the Panax species.

Polymer Adsorbent Purification of Ginsenosides

A purified ginsenoside extract from the Panax species feedstock may be obtained by contacting an aqueous extract of a ginseng feedstock is with a solid polymer affinity adsorbent resin to adsorb the active ginsenosides contained in the aqueous extract onto the adsorbent resin. The bound ginsenosides are then eluted by methods taught herein. Prior to eluting the ginsenosides, the polymer adsorbent resin with the ginsenosides adsorbed thereon may be separated from the remainder of the extract in any convenient manner, preferably, passing the extract through a column containing the resin.

A variety of polymer adsorbent resins can be used to purify the ginsenosides, including, but not limited to, Amberlite XAD-2 (Rohm and Hass), Duolite S-30 (Diamond Alkai Co.), or ADS-8 (Nankai University, Tianjin, China). ADS-8 has high affinity for the triterpene saponin ginsenosides. The ADS-8 resin beads, particle size 0.5-0.6 mm, are a polystyrene copolymer with an ester group. It is believed that the polystyrene adsorbs chemical compounds by hydrophobic interactions between the highly hydrophobic surface of the polystyrene and the hydrophobic sites or sorbates. Ester groups are used for adsorption of chemicals by hydrogen bonding interactions. These two interactions work together to achieve a high selectivity for bonding of the triterpene saponin ginsenosides. Since hydrophobic interaction is one of the driving forces in this separation, an aqueous solution free of alcohol is the solvent used to contain the chemical constituents that are to be adsorbed. An alcohol is then used as the de-adsorption agent.

Although various eluants may be employed to recover the ginsenosides from the polymer adsorbent resin, in one aspect of the present invention, the eluant comprises a low molecular weight alcohol, including, but not limited to, methanol, ethanol, or propanol. In a second aspect, the eluant comprises a low molecular weight alcohol and water. In another aspect, the eluant comprises a low molecular alcohol in an admixture with another organic solvent. In a further aspect, the eluant comprises a low molecular weight alcohol, a second organic solvent, and water.

The Panax species feedstock may have undergone one or more preliminary processes including, but not limited to, the processes for removing essential oils or solvent leaching steps, shown in FIGS. 1 and 2, prior to contacting the aqueous ginsenoside containing extract with the polymer adsorbent resin.

Using polymer adsorbent resins as taught in the present invention results in highly purified ginsenoside extracts of the Panax species that are free of other chemical constituents which are normally present in natural plant material or in available commercial extraction products. For example, the processes taught in the present invention can result in purified ginsenoside extracts that contain total ginsenosides in excess of 90% by dry mass weight. Using the methods taught herein, it is possible to achieve a purified ginsenoside extract fraction of greater than 95% as measured by % mass.

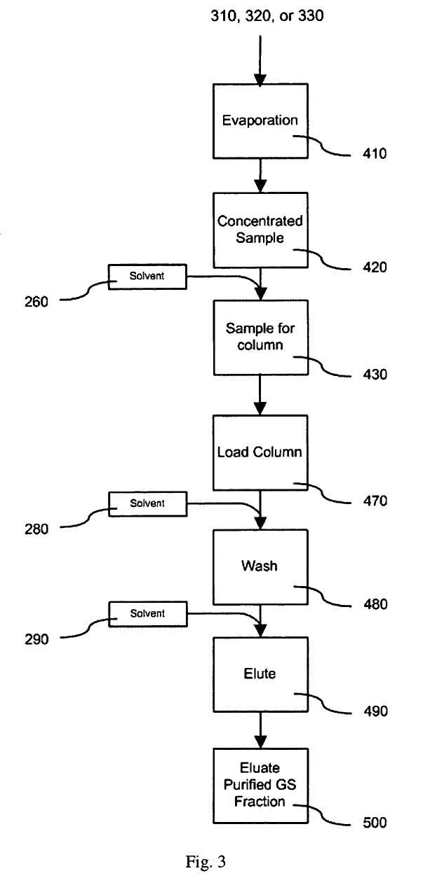

A generalized description of the extraction and purification of the ginsenosides from the rhizome of the Panax species using polymer affinity adsorbent resin beads is diagrammed in FIG. 3. The feedstock for this extraction process may be the hydroalcoholic solutions containing the ginsenosides from FIG. 2, 310, 320, 330. The alcohol is evaporated from this solution 420 and then diluted with distilled water 260 to the original volume to keep the triterpene saponin concentration in this aqueous solution unchanged 420. The appropriate weight of adsorbsent resin beads (50-75 mg of ginsenosides per gram of adsorbent resin) is washed with water and ethanol before and after being loaded into a column. The ginsenoside containing aqueous solution 430 is then loaded onto the column 470 at a flow rate of 2 to 4 bed Volume/hour. Once the column is fully loaded, the column is washed with water 280 at a flow rate of 50 ml/hour to remove any impurities from the adsorbed ginsenosides 480. Elution of the adsorbed ginsenosides 490 is accomplished with ethanol/water (4/1) as an eluting solution 290 at a flow rate of 50 ml/hour and the elution curve recorded for the extract. The eluate 500 consisting of the purified ginsenoside fraction was analyzed using HPLC. Result from individual experiments can be found in Examples 22 through 25, Tables 13 through 20, and FIGS. 9 through 12 (HPLC chromatographs).

Polysaccharide Extraction Process

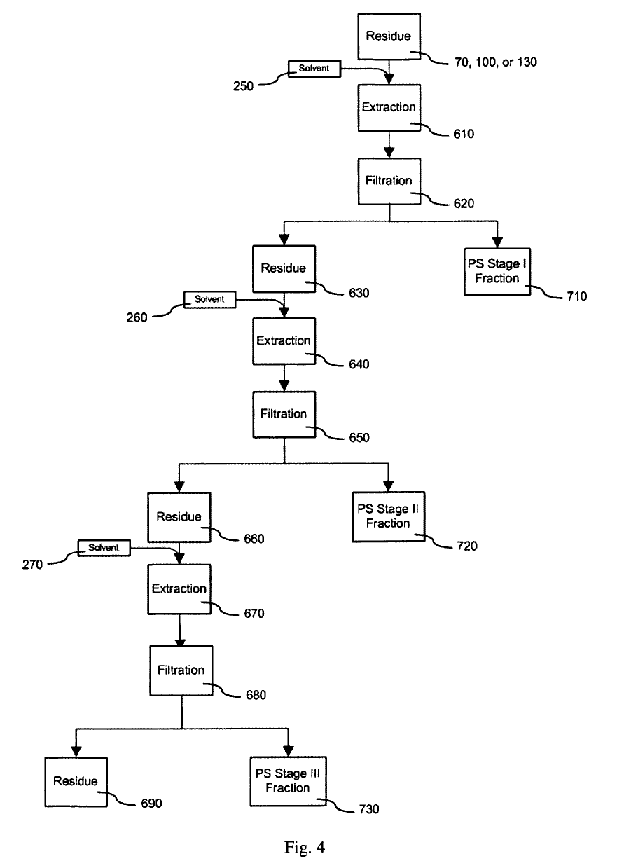

A generalized description of the extraction of the polysaccharide fraction from the rhizome of the Panax species using water solvent leaching and ethanol precipitation processes is diagramed in FIG. 4. The feedstock 70, 100, 130 is the residue from the solvent leaching extraction of the ginsenoside (FIG. 2). The solvent is distilled water 250, 260, 270. The residue feedstock may be extracted multiple times in hot aqueous solutions, for example, water, at approximately 100° C., for at least one hour, for at least two hours, for at least three hours, in an extraction vessel. FIG. 4 shows 3 extractions three times with water at approximately 100° C. The amount of water is generally the same for the first extractions and less for the last extractions. For example, the volume of water for the first extraction 610 and second extraction 640 was 15 ml/gm of feedstock residue and for the third extraction 670 was 10 ml/gm of feedstock residue. After each stage of boiling water leaching the resulting fluid extract is filtered 620, 650, 680. The filtrate is collected as product 710, 720, 730 and measured for volume and solid content (dry mass weight). The extraction residue material retained by the filter in the first stage 260 and then may be used as a feedstock for the second stage of extraction using the same methods, and the process may be repeated in a third stage 670. The residue 690 after the third stage is discarded. Interestingly, the extraction products 710, 720, 730 can be shown to be highly purified polysaccharides (about 99% pure Panax species water soluble, ethanol insoluble polysaccharides) based on a variety of tests which are discussed in Example 30.

The various extract fractions are dried and stored separately for later recombination into a wide variety of nutraceutical and pharmaceutical formulations derived from Panax species extraction products.

Compositions of the present invention comprise extracts of Panax species compositions comprising an essential oil composition, a ginsenoside composition or a polysaccharide composition or combination of one or more of these compositions. Compositions of the present invention may also comprise combinations of one or more ginsenoside compositions taught herein. Compositions of the present invention may also comprise combinations of one or more polysaccharide compositions taught herein. Compositions of the present invention may also comprise combinations of one or more essential oil extraction compositions taught herein.

In one aspect, compositions comprise an essential oil composition comprising an extract from a Panax species made by the methods taught in the present invention. In another aspect, the compositions comprise an essential oil composition comprising an extract from at least two Panax species, wherein an extract is prepared according to the methods of the present from each of at least two Panax species and an extract from each of the at least two Panax species is combined in specific ratios to each other.

In one embodiment, the present invention comprises an essential oil composition prepared from P. notoginseng, wherein the composition comprises the compounds characterized by HPLC spectrograph depicted in FIG. 5. The composition depicted in FIG. 5 has the following characteristic peaks detected by absorbance at 254 nm and eluting at 5.163, 5.760, 6.432, 7.424, 8.107, 8.832, 9.141, 9.643, 10.635, 11.221, 12.000, 12.651, 13.888, 13.995, 14.624, 15.467, 16.064, 16.800, 17.547, 18.112, 18.507, 18.061, 19.296, 19.669, 21.195, 21.781, 22.325, 22.923, 23.797, 24.480, 26.165, 27.808, 28.213, 28.757, 29.237, 30.496, 31.328, 33.408, 34.848, 35.701, 35.938, 37.760, 38.923, 39.285, 40.107, 40.469, 41.088, 42.165, 43.040, 43.488, 44.629, 45.163, 47.659, and 49.461 minutes when the essential oil composition is analyzed under the conditions described in Example 18. The peaks are indicated respectively by reference numbers 1010, 1011, 1012, 1013, 1014, 1015, 1016, 1017, 1018, 1019, 1020, 1021, 1022, 1023, 1024, 1025, 1026, 1027, 1028, 1029, 1030, 1031, 1032, 1033, 1034, 1035, 1036, 1037, 1038, 1039, 1040, 1041, 1042, 1043, 1044, 1045, 1046, 1047, 1048, 1049, 1050, 1051, 1052, 1053, 1054, 1055, 1056, 1057, 1058, 1059, 1060, 1061, 1062, and 1063.

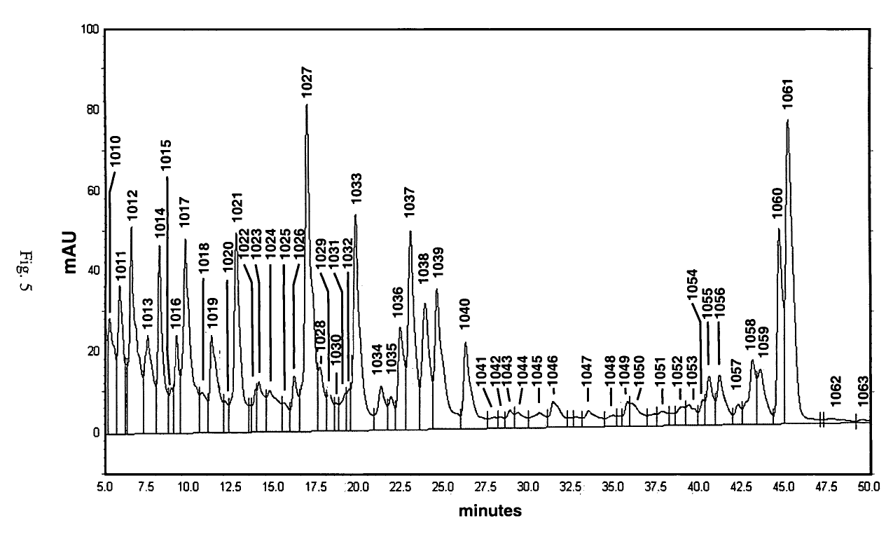

In another embodiment the present invention comprises an essential oil composition prepared from P. quinquefolius, wherein the composition comprises the compounds characterized by BPLC spectrograph depicted in FIG. 6. The composition depicted in FIG. 6 has the following characteristic peaks detected by absorbance at 254 nm and eluting at 5.483, 5.877, 6.123, 6.368, 7.029, 7.691, 8.608, 9.099, 9.792, 10.069, 10.571, 11.339, 11.925, 12.853, 13.195, 13.867, 14.624, 14.860, 15.243, 15.669, 16.288, 16.939, 17.269, 17.537, 18.069, 18.613, 19.925, 21.045, 21.536, 22.357, 22.709, 23.179, 24.363, 24.832, 26.112, 26.869, 27.424, 28.053, 29.301, 30.138, 30.319, 31.051, 31.477, 32.096, 33.675, 34,507, 34.773, 36.075, 36.672, 37.739, 38.155, 38,901, 39.328, 40.000, 40.971, 41.472, 41.835, 42.304, 43.509, 46.325, 46.859, and 48.021 minutes when the essential oil composition is analyzed under the conditions described in Example 19. The peaks are indicated respectively by reference numbers 2010, 2011, 2012, 2013, 2014, 2015, 2016, 2017, 2018, 2019, 2020, 2021, 2022, 2023, 2024, 2025, 2026, 2027, 2028, 2029, 2030, 2031, 2032, 2033, 2034, 2035, 2036, 2037, 2038, 2039, 2040, 2041, 2042, 2043, 2044, 2045, 2046, 2047, 2048, 2049, 2050, 2051, 2052, 2053, 2054, 2055, 2056, 2057, 2058, 2059, 2060, 2061, 2062, 2063, 2064, 2065, 2066, 2067, 2068, 2069, 2070, and 2072.

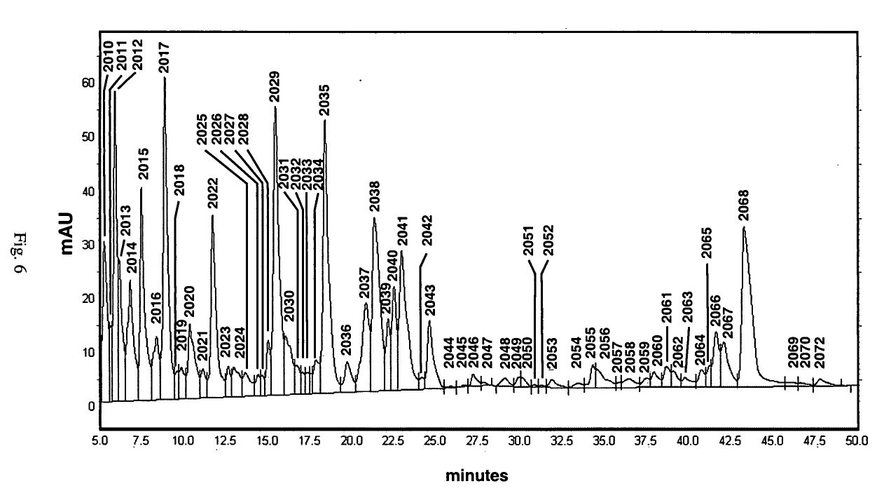

In a further embodiment the present invention comprises an essential oil composition prepared from white ginseng (P. ginseng), wherein the composition comprises the compounds characterized by HPLC spectrograph depicted in FIG. 7. The composition depicted in FIG. 7 has the following characteristic peaks detected by absorbance at 254 nm and eluting at 5.163, 5.621, 5.920, 6.261, 6.485, 7.051, 7.200, 7.659, 8.085, 8.853, 9.333, 10.069, 10.315, 10.912, 11.925, 12.245, 13.749, 14.635, 15.563, 16.352, 17.141, 18.133, 18.667, 18.869, 19.253, 20.267, 20.971, 21.739, 22.059, 22.699, 24.395, 25.707, 26.208, 26.624, 27.168, 28.341, 28.821, 29.813, 30.549, 30.933, 31.669, 33.045, 34.133, 34.464, 35.339, 36.907, 37.525, 38.005, 38.848, 40.213, 40.821, 41.589, 41.771, 42.400, 42.997, 45.568, and 46.165 minutes when the essential oil composition is analyzed under the conditions described in Example 20. The peaks are indicated respectively by reference numbers 3010, 3011, 3012, 3013, 3014, 3015, 3016, 3017, 3018, 3019, 3020, 3021, 3022, 3023, 3024, 3025, 3026, 3027, 3028, 3029, 3030, 3031, 3032, 3033, 3034, 3035, 3036, 3037, 3038, 3039, 3040, 3041, 3042, 3043, 3044, 3045, 3046, 3047, 3048, 3049, 3050, 3051, 3052, 3053, 3054, 3055, 3056, 3057, 3058, 3059, 3060, 3061, 3062, 3063, 3064, 3065, and 3066.

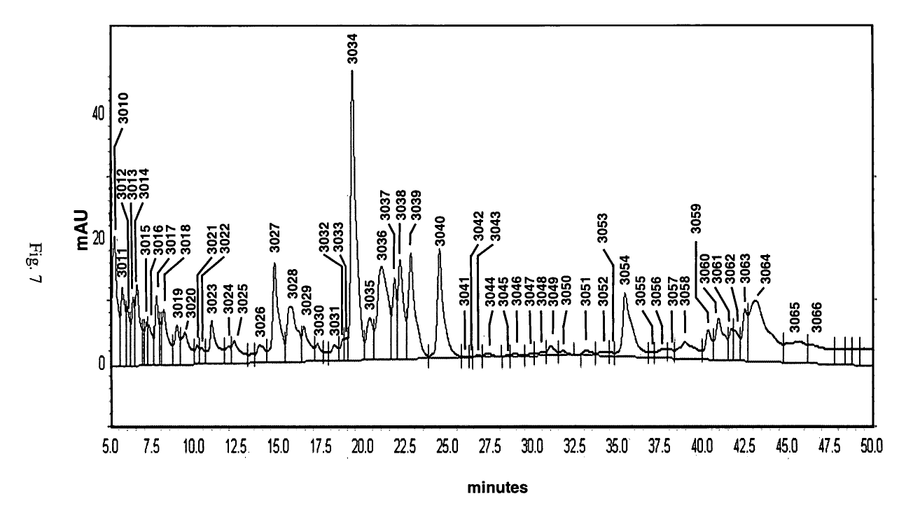

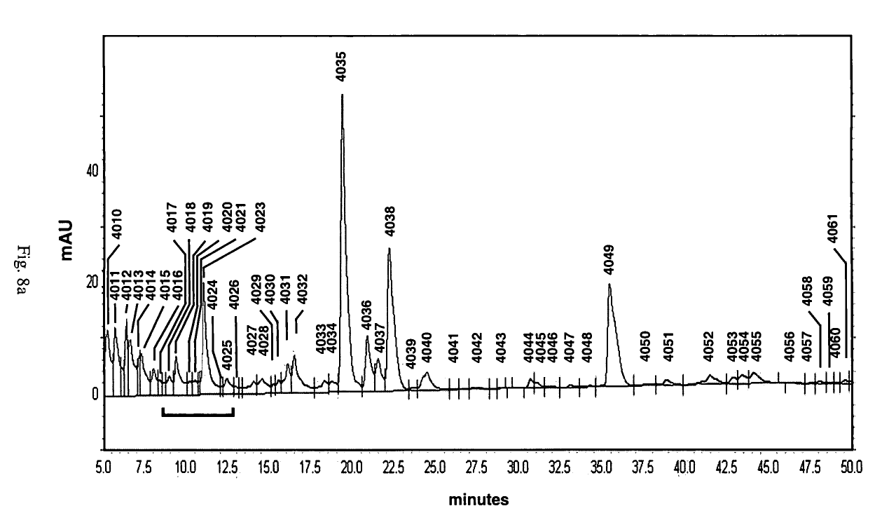

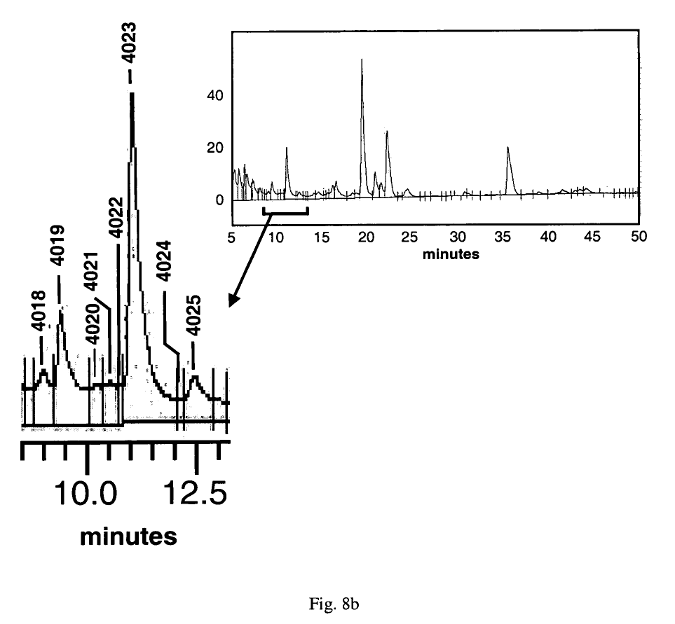

In a further embodiment the present invention comprises an essential oil composition prepared from red ginseng (P. ginseng), wherein the composition comprises the compounds characterized by HPLC spectrograph depicted in FIG. 8. The composition depicted in FIG. 8 has the following characteristic peaks detected by absorbance at 254 nm and eluting at 5.152, 5.611, 6.272, 6.539, 7.072, 7.224, 7.936, 8.277, 8.875, 9.280, 10.091, 10.421, 10.645, 10.923, 12.021, 12.309, 12.853, 13.931, 14.453, 15.147, 15.456, 16.011, 16.395, 18.208, 18.656, 19.307, 20.768, 21.419, 22.101, 23.520, 24.352, 25.888, 27.339, 28.875, 30.603, 30.660, 31.680, 32.981, 34.155, 35.360, 37.963, 38.773, 41.376, 42.784, 43.275, 43.989, 46.251, 47.488, 47.968, 48.619, 48.875, and 49.483 minutes when the essential oil composition is analyzed under the conditions described in Example 21. The peaks are indicated respectively by reference numbers 4010, 4011, 4012, 4013, 4014, 4015, 4016, 4017, 4018, 4019, 4020, 4021, 4022, 4023, 4024, 4025, 4026, 4027, 4028, 4029, 4030, 4031, 4032, 4033, 4034, 4035, 4036, 4037, 4038, 4039, 4040, 4041, 4042, 4043, 4044, 4045, 4046, 4047, 4048, 4049, 4050, 4051, 4052, 4053, 4054, 4055, 4056, 4057, 4058, 4059, 4060, and 4061.

In one aspect, an essential oil fraction of the present invention may comprise a composition comprising one or more of (+)-spathulenol (spathulenol, espatulenol), CAS No. 6750-60-3; caffeine, CAS No. 58-08-2; hexadecanoic acid, CAS No. 57-10-3; (−)-caryophyllene oxide, CAS No. 1139-30-6; ethyl heptanoate, CAS No. 106-30-9; trans,trans-octadeca-9,12-dienoic acid methyl ester, CAS No. 2566-97-4; octadec-9-ynoic acid methyl ester, CAS No. 1120-32-7; phenylacetylene, CAS No. 536-74-3; ethylenethiourea, CAS No. 96-45-7; linoleic acid, CAS No. 60-33-3; 4-methyl 2-enoic acid, CAS No. 10321-71-8; 2-methyl-4-nitroimidazole, CAS No. 696-23-1; 9,12-octadecadienal, CAS No. 26537-70-2; mevinphos, CAS No. 7786-34-7; undec-10-ynoic acid, CAS No. 2777-65-3; falcarinol ((Z)-1,9-heptadecadiene-4,6-diyn-3-ol), CAS No. 21852-80-2; [1R-(1α,4β,4aα,6β,8aα)]-octahydro-4,8a,9,9-tetramethyl-1,6-methano-1(2H)-naphthol, CAS No. 5986-55-0; 4,6-diamino-1,3,5-triazin-2(1H)-one, CAS No. 645-92-1; 2,2′-methyliminodiethanol, CAS No. 105-59-9, dihydrouracil, 504-07-4; stearic acid (octadecanoic acid), CAS No. 57-11-4; 4-nitrophenol, CAS No. 100-02-7; 3-nitrotoluene, CAS No. 99-08-1; 2,3-dihydroxypropyl palmitate, CAS No. 542-44-9; oleic acid, CAS No. 112-80-1; cinnamyl acetate, CAS No. 103-54-8; 7-octenoic acid, CAS No. 18719-24-9; (−)-spathulenol, CAS No. 77171-55-2; 1-methyl-5-nitro-1H-imidazole, CAS No. 3034-42-2; 2-ethyl-2-methyloxirane, CAS No. 30095-63-7; methyl (9E,12E)-octadeca-9,12-dienoate, CAS No. 2566-97-4; sinalbin, CAS No. 27299-07-6, stigmasta-5,22-dien-3-β-ol, CAS No. 83-48-7; (3β,24S)-stigmast-5-en-3-ol, CAS No. 83-47-6; stigmast-5-en-3-β-ol, CAS No. 83-46-5; (3β,24ξ)-stigmast-5-en-3-ol, CAS No. 19044-06-5; 4-methyl-1,4-heptadiene, CAS No. 13857-55-1; 9,12-octadecadienal, CAS No. 26537-70-2; 7,8-epoxyoctene, CAS No. 19600-63-6, 4-nonyne, CAS No. 20184-91-2; 2-cyclopenten-1-undecanoic acid, CAS No. 459-67-6; 3-hydroxy-2-methyl-4-pyrone, CAS No. 118-71-8; pyrogallol, CAS No. 87-66-1; [1aR-(1aα,7α,7aα,7bα)]-1a,2,3,5,6,7,7a,7b-octahydro-1,1,7,7a-tetramethyl-1H-cyclopropa[a]naphthalene, CAS No. 17334-55-3; [1aR-(1aα,4aα,7α,7a β,7bα)]-decahydro-1,1,7-trimethyl-4-methylene-1H-cycloprop[e]azulene, CAS No. 489-39-4; caryophyllene, CAS No. 87-44-5; 1R-(1R*,4Z,9S*)]-4,11,11-trimethyl-8-methylenebicyclo[7.2.0]undec-4-ene, CAS No. 118-65-0; 4-methyl-2-phenyl-2-pentenal, CAS No. 26643-91-4; (Z)-9,17-Octadecadienal, CAS No. 56554-35-9; ethylidenecycloheptane, CAS No. 10494-87-8; octa-1,7-diyne, CAS No. 871-84-1; 3-(phenylmethyl)sydnone, CAS No. 16844-42-1; diisopropyl adipate, CAS No. 6938-94-9; 2,3-dihydroxypropyl palmitate, CAS No. 542-44-9; 9Z,12Z-octadecadienoic acid (2-linoleoyl glycerol), CAS No. 3443-82-1; and, 3-ethenyl-cyclooctene, CAS No. 2213-60-7.

Compositions of the present invention comprise ginsenoside compositions or combinations of one or more ginsenoside extraction compositions taught herein. In one aspect, the compositions comprise a ginsenoside composition comprising an extract from a Panax species, wherein the extract is prepared according the methods taught herein. In another aspect, the compositions comprise a ginsenoside composition comprising an extract from at least two Panax species, wherein an extract is prepared from each of at least two Panax species and an extract from each of the at least two Panax species is combined in a specific ratio to each other.

In an embodiment, the present invention comprises a ginsenoside composition prepared from P. notoginseng, wherein the composition comprises the compounds characterized by HPLC spectrograph depicted in FIG. 9. The composition depicted in FIG. 9 has the following characteristic peaks detected by absorbance at 203 nm and eluting at 8.331, 9.685, 10.720, 12.064, 20.011, 22.699, 25.547, 32.555, 35.712, 37.173, 38.517, 42.091, 42.539, 45.205, 47.072, 50.967, 59.093, and 60.224 minutes when the ginsenoside composition is analyzed under the conditions described in Example 22. The peaks are indicated respectively by reference numbers or specific ginsenoside compound, as appropriate, 5013, Rg1, Re, 5014, 5015, 5016, 5017, 5018, 5021, Rb1, 5022, Rb2, 5023, Rd, 5024, 5025, 5026, and 5027.

In another embodiment the present invention comprises a ginsenoside composition prepared from P. quinquefolius, wherein the composition comprises the compounds characterized by HPLC spectrograph depicted in FIG. 10. The composition depicted in FIG. 10 has the following characteristic peaks detected by absorbance at 203 nm and eluting at 9.984, 10.432, 20.128, 21.803, 26.048, 37.387, 39.008, 41.963, 45.685, and 49.568 minutes when the ginsenoside composition is analyzed under the conditions described in Example 23. The peaks are indicated respectively by reference numbers or specific ginsenoside compound, as appropriate, Rg1, Re, 6010, 6011, 6012, Rb1, Rc, Rb2, Rd, and 6014.

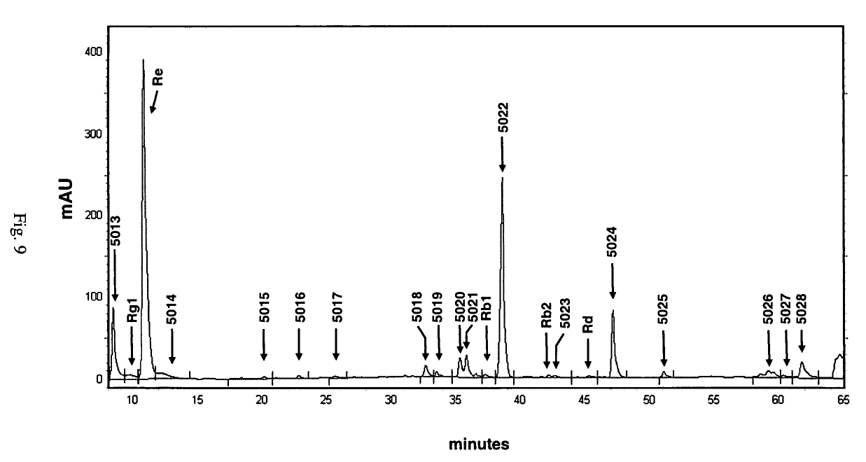

In a further embodiment the present invention comprises a ginsenoside composition prepared from white ginseng (P. ginseng), wherein the composition comprises the compounds characterized by HPLC spectrograph depicted in FIG. 11. The composition depicted in FIG. 11 has the following characteristic peaks detected by absorbance at 203 nm and eluting at 6.635, 7.861, 8.683, 9.024, 10.272, 11.189, 12.971, 16.875, 20.725, 30.528, 32.821, 35.947, 38.549, 40.427, 42.773, and 47.168 minutes when the ginsenoside composition is analyzed under the conditions described in Example 24. The peaks are indicated respectively by reference numbers or specific ginsenoside compound, as appropriate, 7010, 7011, 7012, 7013, Re, 7014, 7016, 7018, 7020, 7023, 7024, 7026, Rb1, Rb2, 7029, and 7032.

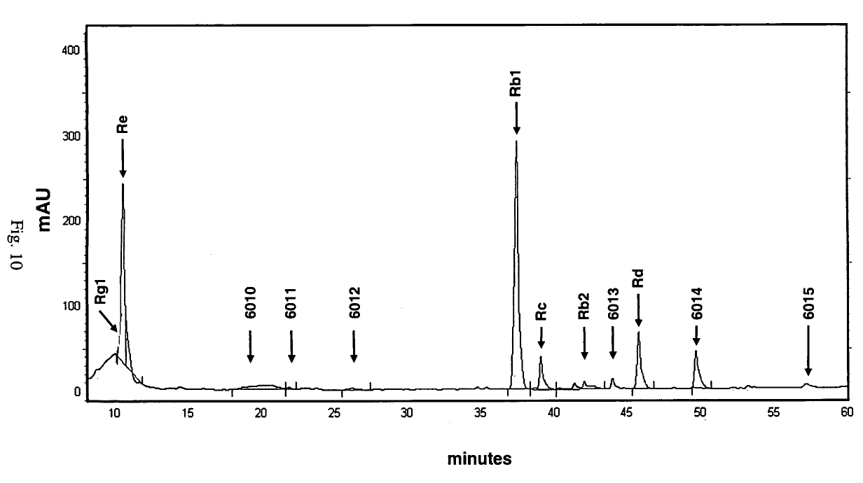

In a further embodiment the present invention comprises a ginsenoside composition prepared from red ginseng (P. ginseng), wherein the composition comprises the compounds characterized by HPLC spectrograph depicted in FIG. 12. The composition depicted in FIG. 12 has the following characteristic peaks detected by absorbance at 203 nm and eluting at 9.888, 11.424, 29.280, 31.563, 34.635, 36.373, 37.152, 38.987, 41.312, 43.904, 45.675, 48.171, 57.109, and 58.720 minutes when the ginsenoside composition is analyzed under the conditions described in Example 25. The peaks are indicated respectively by reference numbers or specific ginsenoside compound, as appropriate, Rg1, 8015, Rf, 8016, 8017, 8019, Rb1, Rc, Rb2, 8022, Rd, 8023, 8024, and 8025.

In making a composition comprising an essential oil composition, a ginsenoside composition or a polysaccharide composition, from about 0.001 ml to about 100 ml of an essential oil fraction, can be used. Additionally, from about 0.001 mg to about 100 mg of a ginsenoside fraction composition can be used. Further, from about 0.001 mg to about 100 mg of the water-soluble fraction can be used. For example, the essential oil composition as embodied by the composition shown in FIG. 5 may be combined with a ginsenoside composition as embodied by the composition shown in FIG. 12.

Many methods are known in the art for removal of alcohol from solution. If it is desired to keep the alcohol for recycling, the alcohol can be removed from the solutions, after extraction, by distillation under normal or reduced atmospheric pressures. The alcohol can be reused. Furthermore, there are also many methods known in the art for removal of water from solutions, either aqueous solutions or solutions from which alcohol was removed. Such methods include, but not limited to, spray drying the aqueous solutions onto a suitable carrier such as, but not limited to, magnesium carbonate or maltodextrin, or alternatively, the liquid can be taken to dryness by freeze drying or refractive window drying.

In performing the previously described extraction methods, it was found that greater than 80% yield by mass weight of the essential oil in the original dried rhizome feedstock of the Panax species can be extracted in the essential oil extract fraction (Step 1). Using the methods of shown in FIG. 2 greater than 98% yield by mass weight of the ginsenoside chemical constituents of the original dried rhizome feedstock of the Panax species can be extracted in the ginsenoside extract fraction. Furthermore, it appears that greater than 99% by weight of the polysaccharide constituents of the original dried rhizome feedstock of the Panax species can be extracted in the polysaccharide fraction. Finally, the methods as taught in the present invention permit the purification (concentration) of the essential oil fraction, ginsenoside fraction, and the polysaccharide fraction to be as high as 99% of the desired chemical constituents (essentially all of the essential oil, triterpene saponin, and polysaccharide chemical constituents present in the original Panax species plant material). The specific extraction environments, rates of extraction, solvents, and extraction technology used often are adjusted depending on the starting chemical constituent profile of the source material and the level of purification desired in the final extraction products. Specific methods as taught in the present invention can be readily adjusted by those skilled in the art using no more than routine experimentation typical for adjusting a process to account for sample variations in starting materials. For example, in a particular lot of P. ginseng (White Ginseng), the initial concentrations of the essential oil, the ginsenosides, and the polysaccharides are determined using methods known to those skilled in the art. One skilled in the art can determine the amount of change from the initial concentration of the ginsenosides, for instance, to the predetermined amounts of ginsenosides for the final extraction product using the extraction methods, as disclosed herein, to reach the desired concentration in the final P. ginseng composition product.

In general, the methods and compositions of the present invention comprise methods for making an extracted Panax species composition having predetermined characteristics. Such an extracted Panax species composition may comprise any one, two, or all three of the three concentrated extract fractions depending on the beneficial biological effect(s) desired for the given product. Typically, a composition containing all three Panax species extraction fractions is generally desired as such novel compositions represent highly purified Panax species extraction products comprising all three biologically beneficial chemical constituents found in the native plant material. Embodiments of the invention comprise methods wherein the predetermined characteristics comprise a predetermined selectively increased concentration of the Panax species' essential oil, ginsenosides, and polysaccharide in separate extraction fractions.

Compositions comprise extracted Panax species plant material or an extracted Panax species composition, or combinations or mixtures of both. Compositions comprise extracted Panax species plant material having a predetermined characteristic or an extracted Panax species composition having a predetermined characteristic. An embodiment of such compositions comprises a predetermined essential oil concentration wherein the predetermined essential oil concentration is a concentration of essential oil that is greater than that which is present in the natural Panax species plant material or conventional Panax species extract products, which can result from the extraction techniques taught herein. For example, a composition may comprise greater than 0.5% wt essential oil. Another embodiment of such compositions comprises a predetermined ginsenoside concentration in the extracted Panax species composition wherein the ginsenoside concentration is greater than that found in the native plant material or conventional Panax species extracts. For example, a composition may comprise P notoginseng ginsenosides at a concentration of greater than 12.0% wt, a composition comprising white ginseng may comprise ginsenosides at a concentration of greater than 5.0% wt, and a composition comprising P. quinquefolius may comprise ginsenosides at a concentration of greater than 8.0% by wt. A further embodiment of such compositions comprises a predetermined polysaccharide concentration substantially increased in relation to that found in natural Panax species dried plant material or conventional Panax species extract products. For example, an extract composition may comprise the water soluble, ethanol insoluble fractions of greater than 50% of P. notoginseng. An embodiment of such compositions comprise predetermined concentrations of the extracted and purified chemical constituent fractions wherein the Panax species essential oil/ginsenoside, essential oil/polysaccharide, and ginsenoside/polysaccharide concentration (% dry weight) profiles (ratios) are greater or less than that found in the natural dried plant material or conventional Panax species extraction products. Alteration of the concentration relationships (chemical profiles) of the beneficial chemical constituents of the individual Panax species permits the formulation of unique or novel Panax species extract composition products designed for specific human conditions or ailments.

The extract compositions of the present invention may have a percent by mass greater or lesser concentrations of total ginsenosides than native Panax species. For example, a composition of the present invention may have a total ginsenoside percent mass that is 2.5 times that of a native Panax species. Further, the compositions of the present invention may greater or lesser amounts, by percent mass, of a polysaccharide or essential oil concentration, when compared to native Panax species. The ratio of essential oil to polysaccharide is greater in the compositions of the present invention than the ratio found in native Panax species. The ratio of total ginsenosides to that of polysaccharides is greater than that found naturally in species of the genus Panax. Compositions of the present invention also comprise ratios of essential oil to that of total ginsenosides where the ratio is less than that found naturally in species of the genus Panax.

According to a further aspect of the invention, the novel extracted Panax species plant material or a novel Panax species extract composition can be further processed to dry, flowable powder. The powder can be used as a dietary supplement that can be added to various edible products. The powder or the final predetermined unique extract compositions of the Panax species are also suitable for use in a rapid dissolve tablet.

According to a particular aspect of the present invention, the extracted Panax species compositions are produced to have a predetermined essential oil, ginsenoside, and polysaccharide concentrations that are greater than that found in the natural plant material or conventional Panax species extract products and/or predetermined novel profiles of the three major bioactive chemical constituents of the Panax species, wherein the ratios (profiles) of the amounts (% dry weight) of essential oil/ginsenoside and/or essential oil/polysaccharide and/or ginsenoside/polysaccharide are greater or less than the chemical constituent profiles found in the natural Panax species plant material or known Panax species extraction products. Such compositions are particularly well suited for delivery in the oral cavity of human subjects, e.g., via a rapid dissolve tablet.

In one embodiment of a method for producing a Panax species extraction powder, a dry extracted Panax species composition is mixed with a suitable solvent, such as but not limited to water or ethyl alcohol, along with a suitable food-grade material using a high shear mixer and then spray air-dried using conventional techniques to produce a powder having grains of very small Panax species extract particles combined with a food-grade carrier.

In a particular example, an extracted Panax species composition is mixed with about twice its weight of a food-grade carrier such as maltodextrin having a particle size of between 100 to about 150 micrometers and an ethyl alcohol solvent using a high shear mixer. Inert carriers, such as silica, preferably having an average particle size on the order of about 1 to about 50 micrometers, can be added to improve the flow of the final powder that is formed. Preferably, such additions are up to 2% by weight of the mixture. The amount of ethyl alcohol used is preferably the minimum needed to form a solution with a viscosity appropriate for spay air-drying. Typical amounts are in the range of between about 5 to about 10 liters per kilogram of extracted Panax species material. The solution of extracted Panax species composition, maltodextrin and ethyl alcohol is spray air-dried to generate a powder with an average particle size comparable to that of the starting carrier material.

In a second embodiment, an extracted Panax species composition and food-grade carrier, such as magnesium carbonate, a whey protein, or maltodextrin are dry mixed, followed by mixing in a high shear mixer containing a suitable solvent, such as water or ethyl alcohol. The mixture is then dried via freeze drying or refractive window drying. In a particular example, extracted Panax species composition material is combined with food grade material about one and one-half times by weight of the extracted Panax species composition, such as magnesium carbonate having an average particle size of about 20 to 200 micrometers. Inert carriers such as silica having an particle size of about 1 to about 50 micrometers can be added, preferably in an amount up to 2% by weight of the mixture, to improve the flow of the mixture. The magnesium carbonate and silica are then dry mixed in a high speed mixer, similar to a food processor-type of mixer, operating at 100's of rpm. The extracted Panax species composition material is then heated until it flows like a heavy oil. Preferably, it is heated to about 50° C. The heated extracted Panax species composition is then added to the magnesium carbonate and silica powder mixture that is being mixed in the high shear mixer. The mixing is continued preferably until the particle sizes are in the range of between about 250 micrometers to about 1 millimeter. Between about 2 to about 10 liters of cold water (preferably at about 4° C.) per kilogram of extracted Panax species composition material is introduced into a high shear mixer. The mixture of extracted Panax species composition, magnesium carbonate, and silica is introduced slowly or incrementally into the high shear mixer while mixing. An emulsifying agent such as carboxymethylcellulose or lecithin can also be added to the mixture if needed. Sweetening agents such as Sucralose or Acesulfame K up to about 5% by weight can also be added at this stage if desired, Alternatively, extract of Stevia rebaudiana, a very sweet-tasting dietary supplement, can be added instead of or in conjunction with a specific sweetening agent (for simplicity, Stevia will be referred to herein as a sweetening agent). After mixing is completed, the mixture is dried using freeze-drying or refractive window drying. The resulting dry flowable powder of extracted Panax species composition material, magnesium carbonate, silica and optional emulsifying agent and optional sweetener has an average particle size comparable to that of the starting carrier and a predetermined extraction Panax species composition.

According to another embodiment, an extracted Panax species composition material is combined with approximately an equal weight of food-grade carrier such as whey protein, preferably having a particle size of between about 200 to about 1000 micrometers. Inert carriers such as silica having a particle size of between about 1 to about 50 micrometers, or carboxymethylcellulose having a particle size of between about 10 to about 100 micrometers can be added to improve the flow of the mixture. Preferably, an inert carrier addition is no more than about 2% by weight of the mixture. The whey protein and inert ingredient are then dry mixed in a food processor-type of mixer that operates over 100 rpm. The Panax species extraction composition material is heated until it flows like a heavy oil (preferably heated to 50°C.). The heated Panax species extraction composition is then added incrementally to the whey protein and inert carrier that is being mixed in the food processor-type mixer. The mixing of the Panax species extraction composition and the whey protein and inert carrier is continued until the particle sizes are in the range of about 250 micrometers to about 1 millimeter. Next, 2 to 10 liters of cold water (preferably at about 4° C.) per kilogram of the paste mixture is introduced in a high shear mixer. The mixture of Panax species extraction composition, whey protein, and inert carrier is introduced incrementally into the cold water containing high shear mixer while mixing. Sweetening agents or other taste additives of up to 5% by weight can be added at this stage if desired. After mixing is completed, the mixture is dried using freeze drying or refractive window drying. The resulting dry flowable powder of Panax species extraction composition, whey protein, inert carrier and optional sweetener has a particle size of about 150 to about 700 micrometers and an unique predetermined Panax species extraction composition.

In a further embodiment, a predetermined Panax species extraction composition is dissolved in a SFE CO2 fluid which is then absorbed onto a suitable food-grade carrier such as maltodextrin, dextrose, or starch. Preferably, the SFE CO2 is used as the solvent. Specific examples include starting with a novel extracted Panax species composition and adding from one to one and a half times the extracted Panax species material by weight of the food-grade carrier having a particle size of between about 100 to about 150 micrometers. This mixture is placed into a chamber containing mixing paddles and which can be pressurized and heated. The chamber is pressurized with CO₂ to a pressure in the range between 1100 psi to about 8000 psi and set at a temperature in the range of between about 20° C. to about 100°C. The exact pressure and temperature are selected to place the CO₂ in a supercritical fluid state. Once the C0₂ in the chamber is in the supercritical state, the Panax species extraction composition is dissolved. The mixing paddles agitate the carrier powder so that it has intimate contact with the supercritical CO₂ that contains the dissolved Panax species extract material. The mixture of supercritical CO₂, dissolved Panax species extraction material, and the carrier powder is then vented through an orifice in the chamber which is at a pressure and temperature that does not support the supercritical state for the CO₂. The CO_{2 is thus dissipated as a gas. The resulting powder in the collection vessel is the carrier powder impregnated with the predetermined novel} Panax species extraction composition. The powder has an average particle size comparable to that of the starting carrier material. The resulting powder is dry and flowable. If needed, the flow characteristics can be improved by adding inert ingredients to the starting carrier powder such as silica up to about 2% by weight as previously discussed.

In the embodiments where the extract composition of the Panax species with a predetermined composition or profile is to be included into a oral fast dissolve tablet as described in U.S. Pat. No. 5,298,261, the unique extract can be used “neat”, that is, without any additional components which are added later in the tablet forming process as described in the patent cited. This method, then obviates the necessity to take the unique Panax species extract composition to a dry flowable powder that is then used to make the tablet.

Once a dry Panax species extraction composition powder is obtained, such as by the methods discussed herein, it can be distributed for use, e.g., as a dietary supplement or for other uses. In a particular embodiment, the novel Panax species extraction composition powder is mixed with other ingredients to form a tableting composition of powder which can be formed into tablets. The tableting powder is first wet with a solvent comprising alcohol, alcohol and water, or other suitable solvents in an amount sufficient to form a thick doughy consistency. Suitable alcohols include, but not limited to, ethyl alcohol, isopropyl alcohol, denatured ethyl alcohol containing isopropyl alcohol, acetone, and denatured ethyl alcohol containing acetone. The resulting paste is then pressed into a tablet mold. An automated tablet molding system, such as described in U.S. Pat. No. 5,407,339, can be used. The tablets can then be removed from the mold and dried, preferably by air-drying for at least several hours at a temperature high enough to drive off the solvent used to wet the tableting powder mixture, typically between about 70° C. to about 85° C. The dried tablet can then be packaged for distribution.

Methods and compositions of the present invention comprise compositions comprising unique Panax specie extract compositions in the form of a paste, resin, oil, or powder. An aspect of the present invention comprises compositions of liquid preparations of unique Panax species extract compositions. Liquid preparations for oral administration may take the form of, for example, solutions, syrups or suspensions, or they may be presented as a dry product for reconstitution with water or other suitable vehicle prior to administration. Such liquid preparations may be prepared by conventional means with pharmaceutically acceptable additives such as suspending agents (e.g., sorbitol syrup, methyl cellulose, or hydrogenated edible fats); emulsifying agents (e.g., lecithin or acacia); non-aqueous vehicles (e.g., almond oil, oily esters or ethyl alcohol); preservatives (e.g., methyl or propyl p-hyroxybenzoates or sorbic acid); and artificial or natural colors and/or sweeteners. Compositions of the liquid preparations can be administered to humans or animals in pharmaceutical carriers known to those skilled in the art. Such pharmaceutical carriers include, but are not limited to, capsules, lozenges, syrups, sprays, rinses, and mouthwash.

An aspect of the present invention comprises compositions of a dry powder Panax species extraction composition. Such dry powder compositions may be prepared according to methods disclosed herein and by other methods known to those skilled in the art such as, but not limited to, spray air drying, freeze drying, vacuum drying, and refractive window drying. The combined dry powder compositions can be incorporated into a pharmaceutical carrier such, but not limited to, tablets or capsules, or reconstituted in a beverage such as a tea.

Although the extraction techniques described herein are discussed in terms of Panax species, it should be recognized that compositions of the present invention can also comprise, in the form of a dry flowable powder or other forms, extracts from other plants such as, but not limited to, varieties of turmeric, boswellia, guarana, cherry, lettuce, Echinacia, piper betel leaf, Areca catechu, muira puama, ginger, willow, suma, kava, horny goat weed, ginko bilboa, mate', garlic, puncture vine, arctic root astragalus, eucommia, gastropodia, and uncaria, or pharmaceutical or nutraceutical agents.

The present invention comprises compositions comprising unique Panax species extract compositions in tablet formulations and methods for making such tablets. A tableting powder can be formed by adding about 1% to 40% by weight of the powdered Panax species extract composition, with between 30% to about 80% by weight of a dry water-dispersible absorbant such as, but not limited to, lactose. Other dry additives such as, but not limited to, one or more sweetener, flavoring and/or coloring agents, a binder such as acacia or gum arabic, a lubricant, a disintegrant, and a buffer can also be added to the tableting powder. The dry ingredients are screened to a particle size of between about 50 to about 150 mesh. Preferably, the dry ingredients are screened to a particle size of between about 80 to 100 mesh.

The present invention comprises compositions comprising tablet formulations and methods for making such tablets. Preferably, the tablet has a formulation that results in a rapid dissolution or disintegration in the oral cavity. The tablet is preferably a homogeneous composition that dissolves or disintegrates rapidly in the oral cavity to release the extract content over a period of about 2 seconds or less than 60 seconds or more, preferably about 3 to about 45 seconds, and most preferably between about 5 to about 15 seconds.

Various rapid-dissolve tablet formulations known in the art can be used. Representative formulations are disclosed in U.S. Pat. Nos. 5,464,632; 6,106,861; 6,221,392; 5,298,261; 6,221,392; and 6,200,604; the entire contents of each are expressly incorporated by reference herein. For example, U.S. Pat. No. 5,298,261 teaches a freeze-drying process. This process involves the use of freezing and then drying under a vacuum to remove water by sublimation. Preferred ingredients include hydroxyethylcellulose, such as Natrosol from Hercules Chemical Company, added to between 0.1% and 1.5%. Additional components include maltodextrin (Maltrin, M-500) at between 1% and 5%. These amounts are solubilized in water and used as a starting mixture to which is added the Panax species extraction composition, along with flavors, sweeteners such as Sucralose or Acesulfame K, and emulsifiers such as BeFlora and BeFloraPlus which are extracts of mung bean.

A particularly preferred tableting composition or powder contains about 10% to 60% by of the Panax species extract composition powder and about 30% to about 60% of a water-soluble diluent. Suitable diluents include lactose, dextrose, sucrose, mannitol, and other similar compositions. Lactose is a preferred diluent but mannitol adds a pleasant, cooling sensation and additional sweetness in the mouth. More than one diluent can be used.

A sweetener may also be included, preferably in an amount between 3% to about 40% by weight depending on the desired sweetness. Preferred sweetening substances include sugar, saccharin, sodium cyclamate, aspartame, and Stevia extract used singly or in combination, although other sweeteners could alternatively be used. Flavoring such as mint, cinnamon, citrus (e.g., lemon or orange), mocha, and others can be also included, preferably in an atnount between about 0.001% to about 1% by weight.

A coloring may also be added, including natural and/or synthetic colors which are known in the art as safe and acceptable for use in drug or food products. Coloring, if added, may be added in an amount of between about 0.5% to about 2% by weight.

Typically, this tableting composition will maintain its form without the use of a binder. However, if needed, various binders are suitable and can be added in an amount of between about 5 % to about 15% or as necessary. Preferred binders are acacia or gum arabic. Alternative binders include sodium alginate, extract of Irish moss, panwar gum, ghatti gum, mucilage of isapol husks, carboxymethylcellulose, hydroxyethylcellulose, methylcellulose, polyvinylpyrrolidone, VEEGUM® (R. T. Vanderbilt Co., Inc., Norwalk, Conn.), larch arabogalactan, gelatin, Kappa carrageenan, copolymers of maleic anhydride with ethylene or methyl ether.

A tablet according to this aspect of this invention typically does not require a lubricant to improve the flow of the powder for tablet manufacturing. However, if it is so desired, preferred lubricants include talc, magnesium stearate, calcium stearate, stearic acid, hydrogenated vegetable oils, and carbowax in amount of between about 2% to about homogeneous, the tablet may alternatively be comprised of regions of powdered Panax species 10% by weight.

Similarly, a disintegrant does not appear necessary to produce rapid dissolve tablets using the present tablet composition. However, a disintegrant can be included to increase the speed with which a resulting tablet dissolves in the mouth. If desired, between about 0.5% to about 1% by weight of a disintegrant can be added. Preferred disintegrants include starches, clays, cellulose, algins, gums, crosslinked polymers (including croscarmelose, crospovidone, and sodium starch glycolate), VEEGUM®HV, agar, bentonite, natural sponge, cation exchange resins, aliginic acid, guar gum, citrus pulp, sodium lauryl sulphate in an amount of about 0.5% to about 1% of the total mass of the tablet.

It is also generally unnecessary to buffer the tablet composition. However, a buffer may be beneficial in specific formulations. Preferred buffering agents include mono- and di-sodium phosphates and borates, basic magnesium carbonate and combinations of magnesium and aluminum hydroxide.

In a preferred implementation, the tableting powder is made by mixing in a dry powdered form the various components as described above, e.g., active ingredient (Panax species extract composition), diluent, sweetening additive, and flavoring, etc. An overage in the range of about 10% to about 15% of the active extract of the active ingredient can be added to compensate for losses during subsequent tablet processing. The mixture is then sifted through a sieve with a mesh size preferably in the range of about 80 mesh to about 100 mesh to ensure a generally uniform composition of particles.

The tablet can be of any desired size, shape, weight, or consistency. The total weight of the Panax species extract composition in the form of a dry flowable powder in a single oral dosage is typically in the range of about 40 mg to about 600 mg. An important consideration is that the tablet is intended to dissolve in the mouth and should therefore not be of a shape that encourages the tablet to be swallowed. The larger the tablet, the less it is likely to be accidentally swallowed, but the longer it will take to dissolve or disintegrate. In a preferred form, the tablet is a disk or wafer of about 0.15 inch to about 0.5 inch in diameter and about 0.08 inch to about 0.2 inch in thickness, and has a weight of between about 160 mg to about 1.200 mg. In addition to disk, wafer or coin shapes, the tablet can be in the form of a cylinder, sphere, cube, or other shapes. Although the tablet is preferably extract composition separated by non-Panax species extract regions in periodic or non-periodic sequences, which can give the tablet a speckled appearance with different colors or shades of colors associated with the Panax species extract regions and the non-Panax species extract region.

Compositions of unique Panax species extract compositions may also comprise Panax species compositions in an amount between about 10 mg and about 750 mg per dose. The essential composition of the novel Panax species extract composition can vary wherein essential oil is in an amount between about 0.1 mg and about 10.0 mg. The total ginsenoside composition of the novel Panax species extract compositions can vary between about 1.0 mg and about 150 mg per dose wherein the % mass weight of the ginsenoside constituents in the unique Panax species extraction composition are greater in relation to the % mass weight of ginsenoside than that found in the natural Panax species plant material or conventional Panax species extracts and beverages. The Panax species polysaccharide composition of the novel Panax species extract composition can vary between about 1.0 mg and about 400 mg wherein the % mass weight of the polysaccharide constituents are substantially increased in relation to the % mass weight of polysaccharides found in the natural Panax species plant material or conventional Panax species extracts or beverages. Finally, the % mass weight ratios of the three principal beneficial bioactive chemical constituents (essential oil, ginsenosides, and polysaccharides) derived from the Panax species may be altered to yield additional novel Panax species extract composition profiles for human oral delivery using the doses ranges mentioned previously.

An exemplary 275 mg tablet contains about 150.0 mg powdered predetermine unique Panax species extract composition, about 12.5 mg extract of Stevia, about 35.5 mg carboxymethylcellulose, and about 77.0 mg of lactose (see Example 1). Additional exemplary formations for 300 mg and 350 mg Panax species extraction composition tablets can be found in Examples 2 and 3.

The present invention comprises methods of using compositions comprising unique Panax species extraction compositions disclosed herein. Methods of providing dietary supplementation are contemplated. Such compositions may further comprise vitamins, minerals and antioxidants. Compositions taught herein can also be used in the methods of treatment of various physiological, psychological, and medical conditions. The standardized, reliable and novel Panax species extraction compositions of the present invention are used to prevent and treat cardiovascular and cerebrovascular disease and hypercholesterolemia. The compositions of the present invention can be used to provide cytoprotection and neural protection which are important to prevention of heart attacks and stroke. The novel Panax species extraction compositions are used to provide powerful antioxidant activity to human and animal cells and cell membranes and protect low density lipoprotein from oxidative damage. Pathologies that are related to oxygen radical damage include, but not limited to, cardiovascular disease, cerebrovascular disease (stroke), arthritis, inflammation, hepatic disorders, HIV, and cancer. The novel Panax species extraction compositions provide inhibition of platelet aggregation which is important to the prevention of heart attacks and stroke. Moreover, the Panax species extraction compositions of the present invention are used to provide immune enhancement which is important protection from infectious diseases, cancer and various pulmonary and hepatic diseases. Panax species extract compositions of the present invention have anti-inflammatory activity and anti-diabetic activity. The novel Panax species extraction compositions are also used to prevent or treat neurodegenerative disease such as Alzheimer's and Parkinson's disease. Furthermore, the novel Panax species extraction compostions are used to enhance memory and cognition, reliever chronic fatigue syndromes, and enhance male erectile function. These and other related pathologies are prevented or treated by administering an effective amount of the novel Panax species extraction compositions of the present invention.

The novel Panax species extraction compositions may be administered daily, for one or more times, for the effective treatment of acute or chronic conditions. One method of the present invention comprises administering at least one time a day a composition comprising Panax species constituent compounds. Methods also comprise administering such compositions more than one time per day, more than two times per day, more than three times per day and in a range from 1 to 15 times per day. Such administration may be continuously, as in every day for a period of days, weeks, months, or years, or may occur at specific times to treat or prevent specific conditions. For example, a person may be administered Panax species extract compositions at least once a day for years to enhance mental focus, cognition, and relieve chronic fatigue, or to prevent cardiovascular disease or stroke.

It must be noted that, as used in this specification and the appended claims, the singular forms “a”, “an”, and “the” include plural referents unless the context clearly dictates otherwise.

All patents, patent applications and references included herein are specifically incorporated by reference in their entireties.

It should be understood, of course, that the foregoing relates only to exemplary embodiments of the present invention and that numerous modifications or alterations may be made therein without departing from the spirit and the scope of the invention as set forth in this disclosure.

Although the exemplary embodiments of the present invention describe in detail methods and compositions for Panax extracts, there are numerous modifications or alterations that may suggest themselves to those skilled in the art for use of the methods and compositions herein for Panax extracts.

The present invention is further illustrated by way of the examples contained herein, which are provided for clarity of understanding. The exemplary embodiments should not to be construed in any way as imposing limitations upon the scope thereof. On the contrary, it is to be clearly understood that resort may be had to various other embodiments, modifications, and equivalents thereof which, after reading the description herein, may suggest themselves to those skilled in the art without departing from the spirit of the present invention and/or the scope of the appended claims.

### EXAMPLES

### Example 1

### Preparation of Essential Oil Fraction from P. notoginseng

30 gm of P. notoginseng rhizome feedstock was ground, passed through either a #8 or #20 mesh sieve, and then the resultant rhizome powders were collected. The ground feedstock was loaded into a 250 ml supercritical fluid extraction (SFE) vessel connected to an Applied Separations Supercritical Fluid Extraction Unit model Spe-ed SFE-2 (Allentown, Pa.). The non-adsorb cotton ball was packed at the top and bottom of the extraction vessel to avoid raw botanical flow together with the CO₂ stream. The oven was preheated to the desired temperature of 89° C. before the packed vessel was loaded. After the vessel was connected to the oven, the extraction system was tested for leakage by pressurizing the system with CO₂ (˜60 bar), and purged. The system was closed and pressurized to the desired pressure of 400 bar using an air-driven liquid CO₂ pump. The system was then left for equilibrium for about 3 minutes. A sampling vial (40 ml) was weighed and connected to the sampling port at room temperature. The extraction was stared by flowing CO@ at a rate of 5.5-6.0 gm/min, which was controlled by a needle valve heated at 90° C. to avoid valve clogging by dry ice during depressurization. The solvent to feedstock ratio utilized was about 17-18/1 and the extraction time was 90 minutes. The total yield of the essential oil fraction from P. notogensing was about 0.3% weight percent versus the weight of the initial feedstock, and the percent weight of the essential oil in this essential oil extract fraction was 100%. The feedstock residue was saved and used in additional extraction steps for extracting the ginsenoside and polysaccharide fractions (see Example 9). The results of this extraction process are shown in Example 18, and FIGS. 5 (HPLC) and 13, 17, 18, 19 (GC-MS).

### Example 2

### Preparation of Essential Oil Fraction from P. quinquefolius

30 gm of P. quinquefoliius rhizome feedstock was ground, passed through either a #8 or #20 mesh sieve, and then the resultant rhizome powders were collected. The ground feedstock was loaded into a 250 ml supercritical fluid extraction (SFE) vessel connected to an Applied Separations Supercritical Fluid Extraction Unit model Spe-ed SFE-2 (Allentown, Pa.). The non-adsorb cotton ball was packed at the top and bottom of the extraction vessel to avoid raw botanical flow together with the CO₂ stream. The oven was preheated to the desired temperature of 89° C. before the packed vessel was loaded. After the vessel was connected to the oven, the extraction system was tested for leakage by pressurizing the system with CO₂ (˜60 bar), and purged. The system was closed and pressurized to the desired pressure of 400 bar using an air-driven liquid CO2 pump. The system was then left for equilibrium for about 3 minutes. A sampling vial (40 ml) was weighed and connected to the sampling port at room temperature. The extraction was stared by flowing CO₂ at a rate of 5.5-6.0 gm/min, which was controlled by a needle valve heated at 90° C. to avoid valve clogging by dry ice during depressurization. The solvent to feedstock ratio utilized was about 17-18/1 and the extraction time was 90 minutes. The total yield of the essential oil fraction from P. quinquefolius was about 0.2% weight percent versus the weight of the initial feedstock, and the percent weight of the essential oil in this essential oil extract fraction was 100%. The feedstock residue was saved and used in additional extraction steps for extracting the ginsenoside and polysaccharide fractions (see FIG. 2 and Example 6). The results of this extraction process are shown in Example 19, and FIGS. 6 (HPLC) and 14, 20, 21, and 22 (GC-MS).

### Example 3

### Preparation of Essential Oil Fraction from White Ginseng (P. ginseng)

30 gm of white ginseng (P. ginseng) rhizome feedstock was ground, passed through either a #8 or #20 mesh sieve, and then the resultant rhizome powders were collected. The ground feedstock was loaded into a 250 ml supercritical fluid extraction (SFE) vessel connected to an Applied Separations Supercritical Fluid Extraction Unit model Spe-ed SFE-2 (Allentown, Pa.). The non-adsorb cotton ball was packed at the top and bottom of the extraction vessel to avoid raw botanical flow together with the CO₂ stream. The oven was preheated to the desired temperature of 89° C. before the packed vessel was loaded. After the vessel was connected to the oven, the extraction system was tested for leakage by pressurizing the system with CO₂ (˜60 bar), and purged. The system was closed and pressurized to the desired pressure of 400 bar using an air-driven liquid CO₂ pump. The system was then left for equilibrium for about 3 minutes. A sampling vial (40 ml) was weighed and connected to the sampling port at room temperature. The extraction was stared by flowing CO₂ at a rate of 5.5-6.0 gm/min, which was controlled by a needle valve heated at 90° C. to avoid valve clogging by dry ice during depressurization. The solvent to feedstock ratio utilized was about 17-18/1 and the extraction time was 90 minutes. The total yield of the essential oil fraction from P. quinquefolius was about 0.5% weight percent versus the weight of the initial feedstock, and the percent weight of the essential oil in this essential oil extract fraction was 100%. The feedstock residue was saved and used in additional extraction steps for extracting the ginsenoside and polysaccharide fractions (see FIG. 2 and Example 7). The results of this extraction process are shown in Example 20, and FIGS. 7 (HPLC) and 15, 23, 24, 25, and 26 (GC-MS).

### Example 4

### Preparation of Essential Oil Fraction from Red Ginseng (P. ginseng)

30 gm of red ginseng (P. ginseng) rhizome feedstock was ground, passed through either a #8 or #20 mesh sieve, and then the resultant rhizome powders were collected. The ground feedstock was loaded into a 250 ml supercritical fluid extraction (SFE) vessel connected to an Applied Separations Supercritical Fluid Extraction Unit model Spe-ed SFE-2 (Allentown, Pa.). The non-adsorb cotton ball was packed at the top and bottom of the extraction vessel to avoid raw botanical flow together with the CO_{2 stream. The oven was preheated to the desired temperature of} 89° C. before the packed vessel was loaded. After the vessel was connected to the oven, the extraction system was tested for leakage by pressurizing the system with CO₂ (˜60 bar), and purged. The system was closed and pressurized to the desired pressure of 400 bar using an air-driven liquid CO₂ pump. The system was then left for equilibrium for about 3 minutes. A sampling vial (40 ml) was weighed and connected to the sampling port at room temperature. The extraction was stared by flowing CO₂ at a rate of 5.5-6.0 gm/min, which was controlled by a needle valve heated at 90° C. to avoid valve clogging by dry ice during depressurization. The solvent to feedstock ratio utilized was about 17-18/1 and the extraction time was 90 minutes. The total yield of the essential oil fraction from red ginseng was about 0.4% weight percent versus the weight of the initial feedstock, and the percent weight of the essential oil in this essential oil extract fraction was 100%. The feedstock residue was saved and used in additional extraction steps for extracting the ginsenoside and polysaccharide fractions (see FIG. 2 and Example 8). The results of this extraction process are shown in Example 21, and FIGS. 8 (HPLC) and 16, 27, 28, 29, 30, and 31 (GC-MS).

### Example 5

### Preparation of Ginsenoside Fraction from P. notoginseng

The residue of the 30 gm of ground rhizome (mesh #20) after the essential oil was extracted (see Example 1, Step 1) from P. notoginseng, was extracted using a three stage solvent “leaching” process. In this method, the residue of the essential oil extraction (Step 1, Example 1, FIG. 1) and 200 ml of extraction solvent (63% ethanol in water) was loaded into four flasks heated in a water bath (50-60° C.) with stirring. The extraction was carried out for two hours. The resultant fluid extract was filtered using a Fisher P8 20 μm filter. The filtrate was collected as product and was measured for volume and solid content (dry mass as measured in grams). The extraction residue, material retained on the 20 micron filter, was then used as a feedstock for the second stage of extraction using the same methods, and the process was repeated in the third stage. The residue was saved and used for the Step 4 extraction of the P. notoginseng polysaccharide fraction (Example 13, FIG. 4). The results are tabulated in Tables 5 and 6,

TABLE 5
Three Stage Solvent Leaching Method for P. notoginseng

| | | | | Extrac- | Ex- | | Ginseno- |
|---|---|---|---|---|---|---|---|
| | Extrac- | Feed- | Sol- | tion | tracted | | side |
| | tion | stock | vent | Time | Dry Wt | Yield | Content |
| Stage | Solvent | (gm) | (ml) | (hr) | (gm) | (%) | (%) |
| I | 63% EtOH | 30 | 200 | 2 | 6.3 | 21.01 | 57.75 |
| II | 63% EtOH | — | 200 | 2 | 0.87 | 2.90 | 35.84 |
| III | 63% EtOH | — | 200 | 2 | 0.22 | 0.73 | 20.59 |

TABLE 6
Distribution of Seven Major Ginsenosides
in extracts of from P. notoginseng

| | | | | | | | | | GS |
|---|---|---|---|---|---|---|---|---|---|
| | Rg1 | Re | Rf | Rb1 | Rc | Rb2 | Rd\ | To- | Yield |
| Stage | (%) | (%) | (%) | (%) | (%) | (%) | (%) | tal* | (%)** |
| Root | 7.00 | 1.14 | 0.06 | 2.73 | 0.03 | 0.09 | 0.74 | 12.26 | |
| I | 31.13 | 5.08 | 0.34 | 12.22 | 0.18 | 0.47 | 3.33 | 52.75 | 90.31 |
| II | 21.90 | 3.48 | 0.00 | 8.34 | 0.00 | 0.00 | 2.11 | 35.84 | 98.77 |
| III | 12.83 | 2.15 | 0.00 | 4.48 | 0.00 | 0.00 | 1.14 | 20.59 | 100.00 |

*The total ginsenosides was calculated based on the seven ginsenoside calibrated. There are some other ginsenoside detected (<5%) but not calibrated due to lack of reference standard. Thus, the total ginsenoside content is somewhat higher than the number cited in the table.
**The total ginsenoside yield in the extracts that were accumulated by adding each extraction stage.

### Example 6

### Preparation of Ginsenoside Fraction from P. quinquefolius

A typical experimental example of Step 2 solvent extraction of the ginsenoside fraction is as follows: the residue of the 30 gm of ground rhizome (mesh # 20) after the essential oil was extracted (see Example 1, Step 1) from P. quinquefolius, was extracted using a three stage solvent “leaching” process. In this method, the residue of the essential oil extraction (Step 1, Example 2, FIG. 1) and 200 ml of extraction solvent (63% ethanol in water) was loaded into four flasks heated in a water bath (50-60° C.) with stirring. The extraction was carried out for two hours. The resultant fluid extract was filtered using a Fisher P8 20 μm filter. The filtrate was collected as product and was measured for volume and solid content (dry mass as measured in grams). The extraction residue, material retained on the 20 micron filter, was then used as a feedstock for the second stage of extraction using the same methods, and the process was repeated in the third stage. The residue was saved and used for the Step 4 extraction of the P. quinquefolius polysaccharide fraction (Example 14, FIG. 4). The results are tabulated in Tables 7 and 8,

TABLE 7
Three Stage Solvent Leaching Method for P. quinquefolius

| | | | | Extrac- | Ex- | | Ginseno- |
|---|---|---|---|---|---|---|---|
| | Extrac- | Feed- | Sol- | tion | tracted | | side |
| | tion | stock | vent | Time | Dry Wt | Yield | Content |
| Stage | Solvent | (gm) | (ml) | (hr) | (gm) | (%) | (%) |
| I | 63% EtOH | 30 | 200 | 2 | 2.35 | 7.83 | 22.10 |
| II | 63% EtOH | — | 200 | 2 | 2.04 | 6.80 | 7.34 |
| III | 63% EtOH | — | 200 | 2 | 0.54 | 0.80 | 5.07 |

TABLE 8
Distribution of Seven Major Ginsenoside
in extracts from P. quinquefolius

| | | | | | | | | | GS |
|---|---|---|---|---|---|---|---|---|---|
| | Rg1 | Re | Rf | Rb1 | Rc | Rb2 | Rd\ | To- | Yield |
| Stage | (%) | (%) | (%) | (%) | (%) | (%) | (%) | tal* | (%)** |
| Root | 0.34 | 0.45 | 0.00 | 1.12 | 0.13 | 0.06 | 0.20 | 2.32 | |
| I | 2.89 | 4.36 | 0.00 | 10.64 | 1.33 | 0.77 | 1.89 | 22.10 | 74.55 |
| II | 1.41 | 1.30 | 0.00 | 3.56 | 0.43 | 0.00 | 0.64 | 7.34 | 96.00 |
| III | 1.22 | 0.89 | 0.00 | 2.44 | 0.00 | 0.00 | 0.48 | 5.07 | 100.00 |

*, **see Table 5.

### Example 7

### Preparation of Ginsenoside Fraction from White Ginseng (P. Ginseng)

A typical experimental example of Step 2 solvent extraction of the ginsenoside fraction is as follows: the residue of the 30 gm of ground rhizome (mesh # 20) after the essential oil was extracted (see Example 1, Step 1) from white ginseng (P. ginseng), was extracted using a three stage solvent “leaching” process. In this method, the residue of the essential oil extraction (Step 1, Example 3, FIG. 1) and 200 ml of extraction solvent (63% ethanol in water) was loaded into four flasks heated in a water bath (50-60° C.) with stirring. The extraction was carried out for two hours. The resultant fluid extract was filtered using a Fisher P8 20 μm filter. The filtrate was collected as product and was measured for volume and solid content (dry mass as measured in grams). The extraction residue, material retained on the 20 micron filter, was then used as a feedstock for the second stage of extraction using the same methods, and the process was repeated in the third stage. The residue was saved and used for the Step 4 extraction of the white ginseng polysaccharide fraction (Example 15, FIG. 4). The results are tabulated in Tables 9 and 10.

TABLE 9
Three Stage Solvent Leaching Method
for White Ginseng (P. ginseng)

| | | | | Extrac- | Ex- | | Ginseno- |
|---|---|---|---|---|---|---|---|
| | Extrac- | Feed- | Sol- | tion | tracted | | side |
| | tion | stock | vent | Time | Dry Wt | Yield | Content |
| Stage | Solvent | (gm) | (ml) | (hr) | (gm) | (%) | (%) |
| I | 63% EtOH | 30 | 200 | 2 | 7.94 | 26.45 | 9.81 |
| II | 63% EtOH | — | 200 | 2 | 2.48 | 8.25 | 6.06 |
| III | 63% EtOH | — | 200 | 2 | 0.81 | 2.70 | 3.38 |

TABLE 10
Distribution of Seven Major Ginsenoside in
extracts from White Ginseng (P. ginseng)

| | | | | | | | | | GS |
|---|---|---|---|---|---|---|---|---|---|
| | Rg1 | Re | Rf | Rb1 | Rc | Rb2 | Rd\ | To- | Yield |
| Stage | (%) | (%) | (%) | (%) | (%) | (%) | (%) | tal* | (%)** |
| Root | 0.81 | 0.31 | 0.11 | 0.83 | 0.52 | 0.48 | 0.17 | 3.19 | |
| I | 2.45 | 0.89 | 0.34 | 2.51 | 1.63 | 1.48 | 0.51 | 9.81 | 81.46 |
| II | 1.54 | 0.74 | 0.19 | 1.72 | 1.08 | 0.89 | 0.37 | 6.06 | 97.13 |
| III | 1.24 | 0.49 | 0.00 | 0.96 | 0.00 | 0.42 | 0.00 | 3.38 | 100.00 |

*, **see Table 5.

### Example 8

### Preparation of Ginsenoside Fraction from Red Ginseng (P. Ginseng)

A typical experimental example of Step 2 solvent extraction of the ginsenoside fraction is as follows: the residue of the 30 gm of ground rhizome (mesh # 20) after the essential oil was extracted (see Example 1, Step 1) from red ginseng (P. ginseng), was extracted using a three stage solvent “leaching” process. In this method, the residue of the essential oil extraction (Step 1, Example 4, FIG. 1) and 200 ml of extraction solvent (63% ethanol in water) was loaded into four flasks heated in a water bath (50-60° C.) with stirring. The extraction was carried out for two hours. The resultant fluid extract was filtered using a Fisher P8 20 μm filter. The filtrate was collected as product and was measured for volume and solid content (dry mass as measured in grams). The extraction residue, material retained on the 20 micron filter, was then used as a feedstock for the second stage of extraction using the same methods, and the process was repeated in the third stage. The residue was saved and used for the Step 4 extraction of the red ginseng polysaccharide fraction (Example 16, FIG. 4). The results are tabulated in Tables 11 and 12.

TABLE 11
Three Stage Solvent Leaching Method
for Red Ginseng (P. ginseng)

| | | | | Extrac- | Ex- | | Ginseno- |
|---|---|---|---|---|---|---|---|
| | Extrac- | Feed- | Sol- | tion | tracted | | side |
| | tion | stock | vent | Time | Dry Wt | Yield | Content |
| Stage | Solvent | (gm) | (ml) | (hr) | (gm) | (%) | (%) |
| I | 63% EtOH | 30 | 200 | 2 | 8.34 | 28.10 | 4.65 |
| II | 63% EtOH | — | 200 | 2 | 3.40 | 11.34 | 4.54 |
| III | 63% EtOH | — | 200 | 2 | 1.17 | 3.91 | 0.48 |

TABLE 12
Distribution of Seven Major Ginsenoside
in extracts from Red Ginseng

| | | | | | | | | | GS |
|---|---|---|---|---|---|---|---|---|---|
| | Rg1 | Re | Rf | Rb1 | Rc | Rb2 | Rd\ | To- | Yield |
| Stage | (%) | (%) | (%) | (%) | (%) | (%) | (%) | tal* | (%)** |
| Root | 0.56 | 0.19 | 0.07 | 0.40 | 0.26 | 0.25 | 0.10 | 1.84 | |
| I | 1.42 | 0.50 | 0.16 | 1.04 | 0.59 | 0.72 | 0.27 | 4.65 | 71.00 |
| II | 1.33 | 0.43 | 0.19 | 0.97 | 0.86 | 0.47 | 0.29 | 4.54 | 98.98 |
| III | 0.39 | 0.10 | 0.00 | 0.00 | 0.00 | 0.00 | 0.00 | 0.49 | 100.00 |

### Example 9

### Polymer Adsorbent Purification of Ginsenoside Fraction from P. notoginseng

In a typical experiment (Step 3, FIG. 3), the two stage solvent leaching ginsenoside extract fraction obtained from the P. notoginseng original 30 gm feedstock was first evaporated under reduced atmospheric pressure to remove the ethanol and then diluted with water to the original volume to keep the triterpene saponin concentration unchanged. 40 gm of the polymer adsorbent resin ADS-8 (Nankai University, Tianjin, China) was washed with water and ethanol before and after being loaded into a column (50 cm L×1 cm ID, ˜40 cm3). The ginsenoside aqueous extract fraction was loaded onto the column at a flow rate of 80 to 100 ml/hr. The optimal flow rate through the resin bed was in the range of 2 to 4 bed volume/hr. The volume and concentration of the solution that passed through the polymer adsorbent resin bed was measured and recorded so as to determine the break-through curve. Once the column was fully loaded, the column was washed with 400ml of water at a flow rate of 50 ml/hour to remove the impurities from the adsorbed ginsenosides. Elution of the ginsenosides was then accomplished with 150 ml of ethanol/water (4/1) as an eluting solvent at a flow rate of 50 ml/hr and the elution curve was recorded. For the extract, the loading capacity of the adsorbent resin ADS-8 was about 50 to 75 mg ginsenoside per gram of adsorbent resin. Results from this experiments are tabulated in Tables 13 and 14 and FIGS. 9, 33, and 34 (HPLC analysis for the ginsenosides in the polymer adsorbent resin extract).

TABLE 13
Ginsenoside Yield following column
chromatography using ADS-8 resin.

| | | | | | Ginseno- | Ginseno- |
|---|---|---|---|---|---|---|
| | | | Dry | | side | side |
| | Amount | | Wt | Yield | Content | Yield |
| Stage | (g or ml) | | (g) | (% Wt) | (%) | (%) |
| P. notoginseng root | 30 | g | — | — | 12.27 | |
| EtOH/Water Extract | 375 | ml | 7.1 | 23.7 | 50.70 | 98.0 |
| PA Column Extract | 25 | ml | 2.4 | 8.0 | 94.91 | 64.2 |

TABLE 14
Comparison of Ginsenoside Distribution in the Elution
at Peak with the Ginseng Root Feedstock and the
Solvent Leaching Extract (% dry weight)

| Ginsenoside | | | | | | | | |
|---|---|---|---|---|---|---|---|---|
| Source | Rg1 | Re | Rf | Rb1 | Rc | Rb2 | Rd | Total |
| P notogenside | 7.00 | 1.14 | 0.06 | 2.73 | 0.03 | 0.09 | 0.74 | 12.27 |
| root | | | | | | | | |
| EtOH/Water | 30.01 | 4.88 | 0.30 | 11.75 | 0.16 | 0.41 | 3.18 | 50.70 |
| Extract | | | | | | | | |
| Elution @ | 38.74 | 6.50 | 2.88 | 30.97 | 1.52 | 0.75 | 13.55 | 94.91 |
| 30 ml | | | | | | | | |

### Example 10

### Polymer Adsorbent Purification of Ginsenoside Fraction from P. quinquefolius

In a typical experiment (Step 3, FIG. 3), the two stage solvent leaching ginsenoside extract fraction obtained from the P. quinquefolius original 30 gm feedstock was first evaporated under reduced atmospheric pressure to remove the ethanol and then diluted with water to the original volume to keep the triterpene saponin concentration unchanged. 40 gm of the polymer adsorbent resin ADS-8 (Nankai University, Tianjin, China) was washed with water and ethanol before and after being loaded into a column (50 cm L×1 cm ID, ˜40 cm3). The ginsenoside aqueous extract fraction was loaded onto the column at a flow rate of 80 to 100 ml/hr. The optimal flow rate through the resin bed was in the range of 2 to 4 bed volume/hr. The volume and concentration of the solution that passed through the polymer adsorbent resin bed was measured and recorded so as to determine the break-through curve. Once the column was fully loaded, the column was washed with 400 ml of water at a flow rate of 50 ml/hour to remove the impurities from the adsorbed ginsenosides. Elution of the ginsenosides was then accomplished with 150 ml of ethanol/water (4/1) as an eluting solvent at a flow rate of 50 ml/hr and the elution curve was recorded. For the extract, the loading capacity of the adsorbent resin ADS-8 was about 50 to 75 mg ginsenoside per gram of adsorbent resin. Results from this experiments are tabulated in Tables 15 and 16 and FIGS. 10, 35, and 36 (HPLC analysis for the ginsenosides in the polymer adsorbent resin extract).

TABLE 15
Ginsenoside Yield following column
chromatography using ADS-8 resin.

| | | | | Ginseno- | Ginseno- |
|---|---|---|---|---|---|
| | | Dry | | side | side |
| | Amount | Wt | Yield | Content | Yield |
| Stage | (g or ml) | (g) | (% Wt) | (%) | (%) |
| P. quinquefolius | 30 | | | 2.32 | |
| root | | | | | |
| EtOH/Water Extract | 375 | 4.7 | 14.3 | 15.2 | 96.0 |
| PA Column Extract | 11 | 0.11 | 0.33 | 98.7 | 14.1 |

TABLE 16
Comparison of Ginsenoside Distribution in the Elution
at Peak with the Ginseng Root Feedstock and the
Solvent Leaching Extract (% dry weight)

| Ginsenoside | | | | | | | | |
|---|---|---|---|---|---|---|---|---|
| Source | Rg1 | Re | Rf | Rb1 | Rc | Rb2 | Rd | Total |
| P. | 0.34 | 0.45 | 0.00 | 1.12 | 0.13 | 0.06 | 0.20 | 2.32 |
| quinquefolius | | | | | | | | |
| root | | | | | | | | |
| EtOH/Water | 2.20 | 2.94 | 0.00 | 7.35 | 0.91 | 0.41 | 1.31 | 15.24 |
| Extract | | | | | | | | |
| Elution @ | 25.48 | 1.11 | 1.08 | 60.12 | 1.55 | 2.16 | 6.49 | 98.00 |
| 30 ml | | | | | | | | |

### Example 11

### Polymer Adsorbent Purification of Ginsenoside Fraction from White Ginseng (P. ginseng)

In a typical experiment (Step 3, FIG. 3), the two stage solvent leaching ginsenoside extract fraction obtained from the white ginseng (P. ginseng) original 22 gm feedstock was first evaporated under reduced atmospheric pressure to remove the ethanol and then diluted with water to the original volume to keep the triterpene saponin concentration unchanged. 40 gm of the polymer adsorbent resin ADS-8 (Nankai University, Tianjin, China) was washed with water and ethanol before and after being loaded into a column (50 cm L×1 cm ID, ˜40 cm3). The ginsenoside aqueous extract fraction was loaded onto the column at a flow rate of 80 to 100 ml/hr. The optimal flow rate through the resin bed was in the range of 2 to 4 bed volume/hr. The volume and concentration of the solution that passed through the polymer adsorbent resin bed was measured and recorded so as to determine the break-through curve. Once the column was fully loaded, the column was washed with 400 ml of water at a flow rate of 50 ml/hour to remove the impurities from the adsorbed ginsenosides. Elution of the ginsenosides was then accomplished with 150 ml of ethanol/water (4/1) as an eluting solvent at a flow rate of 50 ml/hr and the elution curve was recorded. For the extract, the loading capacity of the adsorbent resin ADS-8 was about 50 to 75 mg ginsenoside per gram of adsorbent resin. Results from this experiments are tabulated in Tables 17 and 18 and FIGS. 11, 37, and 38 (HPLC analysis for the ginsenosides in the polymer adsorbent resin extract).

TABLE 17
Ginsenoside Yield following column
chromatography using ADS-8 resin.

| | | | | | Ginseno- | Ginseno- |
|---|---|---|---|---|---|---|
| | | | Dry | | side | side |
| | Amount | | Wt | Yield | Content | Yield |
| Stage | (g or ml) | | (g) | (% Wt) | (%) | (%) |
| White Ginseng root | 22 | g | | | 3.19 | |
| EtOH/Water Extract | 250 | | 8.65 | 39.4 | 4.62 | 99.1 |
| PA Column Extract | 20 | ml | 0.16 | 0.73 | 99.3 | 22.7 |

TABLE 18
Comparison of Ginsenoside Distribution in the Elution
at Peak with the Ginseng Root Feedstock and the
Solvent Leaching Extract (% dry weight)

| Ginsenoside | | | | | | | | |
|---|---|---|---|---|---|---|---|---|
| Source | Rg1 | Re | Rf | Rb1 | Rc | Rb2 | Rd | Total |
| White | 0.81 | 0.31 | 0.11 | 0.83 | 0.52 | 0.48 | 0.17 | 3.19 |
| ginseng root | | | | | | | | |
| EtOH/Water | 2.24 | 0.86 | 0.30 | 2.32 | 1.50 | 1.34 | 0.48 | 8.92 |
| Extract | | | | | | | | |
| Elution @ | 31.84 | 7.94 | 5.71 | 22.75 | 15.41 | 10.64 | 3.73 | 99.30 |
| 30 ml | | | | | | | | |

### Example 12

### Polymer Adsorbent Purification of Ginsenoside Fraction from Red Ginseng (P. Ginseng)

In a typical experiment (Step 3, FIG. 3), the two stage solvent leaching ginsenoside extract fraction obtained from the red ginseng (P. ginseng) original 22 gm feedstock was first evaporated under reduced atmospheric pressure to remove the ethanol and then diluted with water to the original volume to keep the triterpene saponin concentration unchanged. 40 gm of the polymer adsorbent resin ADS-8 (Nankai University, Tianjin, China) was washed with water and ethanol before and after being loaded into a column (50 cm L×1 cm ID, ˜40 cm3). The ginsenoside aqueous extract fraction was loaded onto the column at a flow rate of 80 to 100 ml/hr. The optimal flow rate through the resin bed was in the range of 2 to 4 bed volume/hr. The volume and concentration of the solution that passed through the polymer adsorbent resin bed was measured and recorded so as to determine the break-through curve. Once the column was fully loaded, the column was washed with 400 ml of water at a flow rate of 50 ml/hour to remove the impurities from the adsorbed ginsenosides. Elution of the ginsenosides was then accomplished with 150 ml of ethanol/water (4/1) as an eluting solvent at a flow rate of 50 ml/hr and the elution curve was recorded. For the extract, the loading capacity of the adsorbent resin ADS-8 was about 50 to 75 mg ginsenoside per gram of adsorbent resin. Results from this experiments are tabulated in Tables 19 and 20 and FIGS. 12, and 39 (HPLC analysis for the ginsenosides in the polymer adsorbent resin extract).

TABLE 19
Ginsenoside Yield following column
chromatography using ADS-8 resin.

| | | | | | Ginseno- | Ginseno- |
|---|---|---|---|---|---|---|
| | | | Dry | | side | side |
| | Amount | | Wt | Yield | Content | Yield |
| Stage | (g or ml) | | (g) | (% Wt) | (%) | (%) |
| Red Ginseng Root | 22 | g | | | 1.84 | |
| EtOH/Water Extract | 250 | | 8.65 | 39.4 | 4.62 | 99.1 |
| PA Column Extract | 10 | | 0.19 | 0.85 | 96.8 | 44.7 |

TABLE 20
Comparison of Ginsenoside Distribution in the Elution
at Peak with the Ginseng Root Feedstock and the
Solvent Leaching Extract (% dry weight)

| Ginsenoside | | | | | | | | |
|---|---|---|---|---|---|---|---|---|
| Source | Rg1 | Re | Rf | Rb1 | Rc | Rb2 | Rd | Total |
| Red Ginseng | 0.56 | 0.19 | 0.07 | 0.40 | 0.26 | 0.25 | 0.10 | 1.84 |
| Root | | | | | | | | |
| EtOH/Water | 1.39 | 0.48 | 0.17 | 1.02 | 0.67 | 0.64 | 0.24 | 4.62 |
| Extract | | | | | | | | |
| Elution @ | 35.23 | 9.01 | 5.19 | 19.73 | 13.64 | 10.46 | 3.66 | 96.8 |
| 30 ml | | | | | | | | |

### Example 13

### Preparation of Polysaccharide Fraction from P. notoginseng

In a typical experimental protocol, the residue from defatted (essential oil fraction removed, Step 1, FIG. 1, Example 1), de-sapponinized (ginsenoside fraction removed, Step 2, FIG. 2, Example 5) 30 gm ginseng root feedstock powder derived from P. notoginseng was loaded into a 500 ml flask. This residue was extracted three times with boiling water for two hours. The volume of water used in each instance was 500 ml, 500 ml, and then 300 ml. The solutions were freeze dried to determine the solid concentration (Table 21). The extraction yield was 47.22% indicating that almost 100% of the polysaccharides were extracted from the P. notoginseng feedstock. Moreover, the polysaccharide extract fractions were highly purified, probably greater 99% mixtures of polysaccharides of various molecular weights (see Example 30 for analysis).

TABLE 21
Polysaccharide yield from P. notoginseng

| | | Solvent | | | | Polysac- |
|---|---|---|---|---|---|---|
| | Extraction | Vol | Feed | Extraction | Yield | charide |
| Stage | Solvent | (ml) | (g) | Time | (% Dry Wt) | (% Yield) |
| I | Water | 500 | 30 | 2 | 29.80 | 63.1 |
| II | Water | 500 | — | 2 | 10.58 | 22.4 |
| III | Water | 500 | — | 2 | 6.84 | 14.5 |

### Example 14

### Preparation of Polysaccharide Fraction from P. quinquefolius

In a typical experimental protocol, the residue from defatted (essential oil fraction removed, Step 1, FIG. 1, Example 2), de-sapponinized (ginsenoside fraction removed, Step 2, FIG. 2, Example 6) 30 gm ginseng root feedstock powder derived from P. quinquefolius was loaded into a 500 ml flask. This residue was extracted three times with boiling water for two hours. The volume of water used in each instance was 500 ml, 500 ml, and then 300 ml. The solutions were freeze dried to determine the solid concentration (Table 22). The extraction yield was 18.78% indicating that almost 100% of the polysaccharides were extracted from the P. quinquefolius feedstock. Moreover, the polysaccharide extract fractions were highly purified, probably greater 99% mixtures of polysaccharides of various molecular weights (see Example 30 for analysis).

TABLE 22
Polysaccharide yield from P. quinquefolius

| | | Solvent | | | | Polysac- |
|---|---|---|---|---|---|---|
| | Extraction | Vol | Feed | Extraction | Yield | charide |
| Stage | Solvent | (ml) | (g) | Time | (% Dry Wt) | (% Yield) |
| I | Water | 500 | 30 | 2 | 13.88 | 73.9 |
| II | Water | 500 | — | 2 | 3.73 | 19.9 |
| III | Water | 500 | — | 2 | 1.16 | 6.2 |

### Example 15

### Preparation of Polysaccharide Fraction from White Ginseng (P. ginseng)

In a typical experimental protocol, the residue from defatted (essential oil fraction removed, Step 1, FIG. 1, Example 3), de-sapponinized (ginsenoside fraction removed, Step 2, FIG. 2, Example 7) 30 gm ginseng root feedstock powder derived from white ginseng (P. ginseng) was loaded into a 500 ml flask. This residue was extracted three times with boiling water for two hours. The volume of water used in each instance was 500 ml, 500 ml, and then 300 ml. The solutions were freeze dried to determine the solid concentration (Table 21). The extraction yield was 17.44% indicating that almost 100% of the polysaccharides were extracted from the white ginseng feedstock. Moreover, the polysaccharide extract fractions were highly purified, probably greater 99% mixtures of polysaccharides of various molecular weights (see Example 30 for analysis).

TABLE 23
Polysaccharide yield from white ginseng (P. ginseng)

| | | Solvent | | | | Polysac- |
|---|---|---|---|---|---|---|
| | Extraction | Vol | Feed | Extraction | Yield | charide |
| Stage | Solvent | (ml) | (g) | Time | (% Dry Wt) | (% Yield) |
| I | Water | 500 | 30 | 2 | 11.43 | 65.6 |
| II | Water | 500 | — | 2 | 4.01 | 22.9 |
| III | Water | 500 | — | 2 | 2.10 | 11.5 |

### Example 16

### Preparation of Polysaccharide Fraction from Red Ginseng (P. ginseng)

In a typical experimental protocol, the residue from defatted (essential oil fraction removed, Step 1, FIG. 1, Example 4), de-sapponinized (ginsenoside fraction removed, Step 2, FIG. 2, Example 8) 30 gm ginseng root feedstock powder derived from red ginseng (P. ginseng) was loaded into a 500 ml flask. This residue was extracted three times with boiling water for two hours. The volume of water used in each instance was 500 ml, 500 ml, and then 300 ml. The solutions were freeze dried to determine the solid concentration (Table 24). The extraction yield was 47.22% indicating that almost 100% of the polysaccharides were extracted from the red ginseng feedstock. Moreover, the polysaccharide extract fractions were highly purified, probably greater 99% mixtures of polysaccharides of various molecular weights (see Example 30 for analysis).

TABLE 24
Polysaccharide yield from red ginseng (P. ginseng)

| | | Solvent | | | | Polysac- |
|---|---|---|---|---|---|---|
| | Extraction | Vol | Feed | Extraction | Yield | charide |
| Stage | Solvent | (ml) | (g) | Time | (% Dry Wt) | (% Yield) |
| I | Water | 500 | 30 | 2 | 21.30 | 81.5 |
| II | Water | 500 | — | 2 | 3.85 | 14.7 |
| III | Water | 500 | — | 2 | 0.98 | 3.7 |

### Example 17

### HPLC Methods

HPLC analysis of ginseng extracts and fractions was carried out using a Shimadzu SE0405003 HPLC system equipped with the following Shimadzu equipment: a SCL-10AVP System Controller, a DGU-14A four-line vacuum membrane degasser, a FCV-10ALVP low pressure gradient unit, a LC-10ATVP serial plunger solvent delivery unit, a 100 microliter semi-micro mixer, a SIL-10AF fast autosampler, a SPD-M10AVP UV-Vis photodiode array detector, a CTO-10ASvp column oven, Class VP 7.2.1 SP1 chromatography software, and a FRC-10A fraction collector. The column used in Examples 18-25 was a Jupiter 5μ C18 300A, 250×4.6 mm column obtained from Phenomenex, Inc. Solvents used in HPLC methods, including water, ethanol, methanol, and acetonitrile, were HPLC grade and were obtained from Sigma-Aldrich, Inc.

### Example 18

### HPLC Characterization of an Essential Oil Fraction from P. notoginseng

An essential oil fraction from P. notoginseng was prepared as described in Example 1. HPLC analysis was carried using the methods and equipment described in Example 17 with the specific conditions described herein. The essential oil fraction sample was dissolved in HPLC-grade methanol at a concentration of 3 mg/ml. The sample injection volume was 10 μl. The mobile phase components were as follows: mobile phase component A was phosphate buffer, 0.5% phosphoric acid in water, pH 3.5 (“A”); mobile phase component B was methanol (“B”); and mobile phase component C was acetonitrile (“C”). The concentration gradient elution program used was as follows: the initial mobile phase composition comprised on a volume basis 50%, 17%, and 33%, respectively, of A, B, and C; and, at 40 min following sample injection the mobile phase composition comprised on a volume basis 25%, 25%, and 50%, respectively, of A, B, and C. The mobile phase was linear gradient in 490 minutes changing from initially 50:17:33 to 25:25:50 A:B:C and then hold at this condition for another 10 minutes. The total analysis time was 50 min, the mobile phase flow rate was 1 ml per min and the column temperature was controlled at 45° C. Peak detection was at 254 nm. A typical HPLC chromatogram for an essential oil fraction from P. notoginseng is given in FIG. 5. The data from the HPLC chromatogram in FIG. 5 are given Tables 25 and 26.

TABLE 25
HPLC peak retention times for P. notoginseng
essential oil fraction

| | Ret Time |
|---|---|
| Peak No. | (Min) |
| 1010 | 5.163 |
| 1011 | 5.760 |
| 1012 | 6.432 |
| 1013 | 7.424 |
| 1014 | 8.107 |
| 1015 | 8.832 |
| 1016 | 9.141 |
| 1017 | 9.643 |
| 1018 | 10.635 |
| 1019 | 11.221 |
| 1020 | 12.000 |
| 1021 | 12.651 |
| 1022 | 13.888 |
| 1023 | 13.995 |
| 1024 | 14.624 |
| 1025 | 15.467 |
| 1026 | 16.064 |
| 1027 | 16.800 |
| 1028 | 17.547 |
| 1026 | 16.064 |
| 1029 | 18.112 |
| 1030 | 18.507 |
| 1031 | 18.061 |
| 1032 | 19.296 |
| 1033 | 19.669 |
| 1034 | 21.195 |
| 1035 | 21.781 |
| 1036 | 22.325 |
| 1037 | 22.923 |
| 1038 | 23.797 |
| 1039 | 24.480 |
| 1040 | 26.165 |
| 1041 | 27.808 |
| 1042 | 28.213 |
| 1043 | 28.757 |
| 1044 | 29.237 |
| 1045 | 30.496 |
| 1046 | 31.328 |
| 1047 | 33.408 |
| 1048 | 34.848 |
| 1049 | 35.701 |
| 1050 | 35.938 |
| 1051 | 37.760 |
| 1052 | 38.923 |
| 1053 | 39.285 |
| 1054 | 40.107 |
| 1055 | 40.469 |
| 1056 | 41.088 |
| 1057 | 42.165 |
| 1058 | 43.040 |
| 1059 | 43.488 |
| 1060 | 44.629 |
| 1061 | 45.163 |
| 1062 | 47.659 |
| 1063 | 49.461 |

TABLE 26
HPLC analytical data from P. notogenseng essential oil fraction

| Peak | | | | | |
|---|---|---|---|---|---|
| Reference | Retention | Area | Height | Width | Theoretical |
| No. | time (Min) | (mAu · min) | (mAu) | (min) | plate |
| 1010 | 5.163 | 729554 | 28473 | 0.52 | 1577 |
| 1011 | 5.760 | 819443 | 36624 | 0.52 | 1963 |
| 1012 | 6.432 | 1452476 | 51498 | 0.95 | 733 |
| 1013 | 7.424 | 802293 | 24030 | 0.77 | 1487 |
| 1014 | 8.107 | 950323 | 46764 | 0.75 | 1869 |
| 1015 | 8.832 | 165736 | 11151 | 0.27 | 17120 |
| 1016 | 9.141 | 459358 | 24121 | 0.44 | 6906 |
| 1017 | 9.643 | 1464010 | 48140 | 1.11 | 1208 |
| 1018 | 10.635 | 279640 | 9882 | 0.51 | 6958 |
| 1019 | 11.221 | 762472 | 23871 | 0.91 | 2433 |
| 1020 | 12.000 | 139655 | 7655 | 0.32 | 22500 |
| 1021 | 12.651 | 130405 | 49437 | 1.16 | 1903 |
| 1025 | 15.467 | 179407 | 7022 | 0.47 | 17328 |
| 1027 | 16.800 | 2232358 | 80899 | 1.10 | 3732 |
| 1030 | 18.507 | 106150 | 6788 | 0.27 | 75173 |
| 1032 | 19.296 | 136114 | 10101 | 0.23 | 112616 |
| 1033 | 19.669 | 1503880 | 53757 | 1.41 | 3113 |
| 1034 | 21.195 | 334247 | 10696 | 0.79 | 11517 |
| 1035 | 21.781 | 187856 | 8163 | 0.46 | 35872 |
| 1037 | 22.923 | 1315180 | 49311 | 0.80 | 13137 |
| 1038 | 23.797 | 946391 | 31041 | 0.77 | 15282 |
| 1039 | 24.480 | 1218557 | 34747 | 1.62 | 3654 |
| 1040 | 26.165 | 698311 | 21270 | 1.57 | 4444 |
| 1043 | 28.757 | 119531 | 4332 | 0.58 | 39332 |
| 1046 | 31.328 | 277161 | 6243 | 1.14 | 12083 |
| 1048 | 34.848 | 100553 | 2633 | 0.71 | 38544 |
| 1050 | 35.936 | 234739 | 5643 | 0.99 | 21082 |
| 1051 | 37.760 | 128902 | 3483 | 0.68 | 49336 |
| 1054 | 40.107 | 116560 | 6216 | 0.39 | 169212 |
| 1056 | 41.088 | 401550 | 12183 | 1.02 | 25963 |
| 1059 | 43.488 | 438790 | 13497 | 0.97 | 32160 |
| 1060 | 44.629 | 1026165 | 48501 | 0.63 | 80292 |
| 1061 | 45.163 | 2300562 | 75294 | 2.12 | 7261 |

### Example 19

### HPLC Characterization of an Essential Oil Fraction from P. quinquefolius

An essential oil fraction from P. quinquefolius was prepared as described in Example 2. HPLC analysis was carried out as described in Example 18. A typical HPLC chromatogram for an essential oil fraction from P. quinquefolius is given in FIG. 6. The retention times for the designated peaks from the HPLC chromatogram in FIG. 6 are given Table 27. Additional data for representative peaks from the HPLC chromatogram in FIG. 6 are given in Table 28.

TABLE 27
HPLC retention times for P. quinquefolius essential oil fraction

| | Ret Time |
|---|---|
| Peak No. | (Min) |
| 2010 | 5.483 |
| 2011 | 5.877 |
| 2012 | 6.123 |
| 2013 | 6.368 |
| 2014 | 7.029 |
| 2015 | 7.691 |
| 2016 | 8.608 |
| 2017 | 9.099 |
| 2018 | 9.792 |
| 2019 | 10.069 |
| 2010 | 5.483 |
| 2021 | 11.339 |
| 2022 | 11.925 |
| 2023 | 12.853 |
| 2024 | 13.195 |
| 2025 | 13.867 |
| 2026 | 14.624 |
| 2027 | 14.860 |
| 2028 | 15.243 |
| 2029 | 15.669 |
| 2030 | 16.288 |
| 2031 | 16.939 |
| 2032 | 17.269 |
| 2033 | 17.537 |
| 2034 | 18.069 |
| 2035 | 18.613 |
| 2036 | 19.925 |
| 2037 | 21.045 |
| 2038 | 21.536 |
| 2039 | 22.357 |
| 2040 | 22.709 |
| 2041 | 23.179 |
| 2042 | 24.363 |
| 2043 | 24.832 |
| 2044 | 26.112 |
| 2045 | 26.869 |
| 2046 | 27.424 |
| 2047 | 28.053 |
| 2048 | 29.301 |
| 2049 | 30.138 |
| 2050 | 30.319 |
| 2051 | 31.051 |
| 2052 | 31.477 |
| 2053 | 32.096 |
| 2054 | 33.675 |
| 2055 | 34.507 |
| 2056 | 34.773 |
| 2057 | 36.075 |
| 2058 | 36.672 |
| 2059 | 37.739 |
| 2060 | 38.155 |
| 2061 | 38.901 |
| 2062 | 39.328 |
| 2063 | 40.000 |
| 2064 | 40.971 |
| 2065 | 41.472 |
| 2066 | 41.835 |
| 2067 | 42.304 |
| 2068 | 43.509 |
| 2069 | 46.325 |
| 2070 | 46.859 |
| 2072 | 48.021 |

TABLE 28
HPLC data for P. quinquefolius essential oil fraction

| Peak | | | | | |
|---|---|---|---|---|---|
| Reference | Retention | Area | Height | Width | Theoretical |
| No. | time (Min) | (mAu · min) | (mAu) | (min) | plate |
| 2011 | 5.877 | 150013 | 14034 | 0.18 | 17056 |
| 2013 | 6.368 | 430822 | 26350 | 0.43 | 3509 |
| 2017 | 9.099 | 1146303 | 60042 | 0.85 | 1833 |
| 2018 | 9.792 | 67025 | 5204 | 0.22 | 31697 |
| 2020 | 10.571 | 400240 | 13788 | 0.84 | 2534 |
| 2021 | 11.339 | 112153 | 5432 | 0.43 | 11126 |
| 2022 | 11.925 | 763520 | 33956 | 1.08 | 1951 |
| 2023 | 12.853 | 93320 | 5697 | 0.35 | 21577 |
| 2029 | 15.669 | 1290672 | 53585 | 0.80 | 6138 |
| 2031 | 16.639 | 105596 | 5362 | 0.36 | 34180 |
| 2035 | 18.613 | 1203276 | 50697 | 1.25 | 3548 |
| 2036 | 19.925 | 167289 | 5588 | 0.85 | 8792 |
| 2037 | 21.045 | 510534 | 16352 | 0.91 | 8557 |
| 2039 | 22.357 | 217905 | 13254 | 0.34 | 69181 |
| 2040 | 22.709 | 373474 | 19172 | 0.43 | 44625 |
| 2042 | 24.363 | 41101 | 2107 | 0.35 | 77526 |
| 2044 | 26.112 | 6263 | 225 | 0.74 | 19922 |
| 2048 | 29.301 | 48734 | 1562 | 1.02 | 13203 |
| 2053 | 32.096 | 41998 | 1449 | 1.32 | 9460 |
| 2055 | 34.507 | 86605 | 4169 | 0.69 | 40016 |
| 2057 | 36.075 | 16710 | 891 | 0.34 | 180125 |
| 2059 | 37.739 | 45847 | 1617 | 0.61 | 61241 |
| 2063 | 40.000 | 62679 | 1711 | 0.86 | 34613 |
| 2064 | 40.971 | 74440 | 3156 | 0.55 | 88787 |
| 2068 | 43.509 | 1207959 | 29593 | 2.83 | 3782 |
| 2069 | 46.325 | 28406 | 716 | 0.77 | 57912 |

### Example 20

### HPLC Characterization of an Essential Oil Fraction from White Ginseng (P. ginseng)

An essential oil fraction from White Ginseng (P. ginseng) was prepared as described in Example 3. HPLC analysis was carried out as described in Example 18. A typical HPLC chromatogram for an essential oil fraction from White Ginseng (P. ginseng) is given in FIG. 7 The retention times for the designated peaks from the HPLC chromatogram in FIG. 7 are given Table 29. Additional data for representative peaks from the HPLC chromatogram in FIG. 7 are given in Table 30.

TABLE 29
HPLC peak retention times for white ginseng essential oil fraction

| | Ret Time |
|---|---|
| Peak No. | (Min) |
| 3010 | 5.163 |
| 3011 | 5.621 |
| 3012 | 5.920 |
| 3013 | 6.261 |
| 3014 | 6.485 |
| 3015 | 7.051 |
| 3016 | 7.200 |
| 3017 | 7.659 |
| 3018 | 8.085 |
| 3019 | 8.853 |
| 3020 | 9.333 |
| 3021 | 10.069 |
| 3022 | 10.315 |
| 3023 | 10.912 |
| 3024 | 11.925 |
| 3025 | 12.245 |
| 3026 | 13.749 |
| 3027 | 14.635 |
| 3028 | 15.563 |
| 3029 | 16.352 |
| 3030 | 17.141 |
| 3031 | 18.133 |
| 3032 | 18.667 |
| 3033 | 18.869 |
| 3034 | 19.253 |
| 3035 | 20.267 |
| 3036 | 20.971 |
| 3037 | 21.739 |
| 3038 | 22.059 |
| 3039 | 22.699 |
| 3040 | 24.395 |
| 3041 | 25.707 |
| 3042 | 26.208 |
| 3043 | 26.624 |
| 3044 | 27.168 |
| 3045 | 28.341 |
| 3046 | 28.821 |
| 3047 | 29.813 |
| 3048 | 30.549 |
| 3049 | 30.933 |
| 3050 | 31.669 |
| 3051 | 33.045 |
| 3052 | 34.133 |
| 3053 | 34.464 |
| 3054 | 35.339 |
| 3055 | 36.907 |
| 3056 | 37.525 |
| 3057 | 38.005 |
| 3058 | 38.848 |
| 3059 | 40.213 |
| 3060 | 40.821 |
| 3061 | 41.589 |
| 3062 | 41.771 |
| 3063 | 42.400 |
| 3064 | 42.997 |
| 3065 | 45.568 |
| 3066 | 46.165 |

TABLE 30
HPLC data for white ginseng essential oil fraction

| Peak | | | | | |
|---|---|---|---|---|---|
| Reference | Retention | Area | Height | Width | Theoretical |
| No. | time (Min) | (mAu · min) | (mAu) | (min) | plate |
| 3010 | 5.163 | 435461 | 20638 | 0.54 | 1463 |
| 3011 | 5.621 | 218148 | 12460 | 0.36 | 3901 |
| 3014 | 6.485 | 256854 | 12888 | 0.48 | 2921 |
| 3018 | 8.085 | 208736 | 8568 | 0.66 | 2401 |
| 3019 | 8.853 | 120925 | 6129 | 0.45 | 6193 |
| 3020 | 9.333 | 171698 | 4912 | 0.82 | 2073 |
| 3022 | 10.315 | 48577 | 2528 | 0.35 | 13897 |
| 3023 | 10.912 | 213142 | 6700 | 1.11 | 1546 |
| 3024 | 11.925 | 56033 | 2638 | 0.38 | 15757 |
| 3028 | 15.563 | 353363 | 8748 | 0.96 | 4205 |
| 3030 | 17.141 | 65996 | 2690 | 0.54 | 16121 |
| 3032 | 18.667 | 51034 | 3348 | 0.31 | 58016 |
| 3034 | 19.325 | 1047860 | 46490 | 0.99 | 6097 |
| 3036 | 20.971 | 647313 | 14806 | 1.01 | 6898 |
| 3038 | 22.059 | 352675 | 15815 | 0.53 | 27717 |
| 3039 | 22.699 | 480051 | 16849 | 1.32 | 4731 |
| 3040 | 24.395 | 453299 | 17408 | 1.91 | 2610 |
| 3042 | 26.208 | 724 | 66 | 0.23 | 207746 |
| 3046 | 28.821 | 14552 | 483 | 0.89 | 16779 |
| 3050 | 31.669 | 16676 | 602 | 0.87 | 21201 |
| 3053 | 34.464 | 7799 | 496 | 0.29 | 225972 |
| 3055 | 36.907 | 15720 | 818 | 0.33 | 200129 |
| 3056 | 37.525 | 52162 | 1341 | 0.77 | 38000 |
| 3059 | 40.213 | 125450 | 4619 | 0.69 | 54344 |
| 3060 | 40.821 | 224164 | 6508 | 0.84 | 37786 |
| 3064 | 42.997 | 754815 | 9586 | 2.11 | 6644 |
| 3066 | 46.165 | 250921 | 3010 | 1.58 | 13659 |

### Example 21

### HPLC Characterization of an Essential Oil Fraction from Red Ginseng (P. ginseng)

An essential oil fraction from Red Ginseng (P. ginseng) was prepared as described in analysis was carried out as described in Example 18. A typical HPLC chromatogram for an essential oil fraction from Red Ginseng (P. ginseng) is given in FIG. 8. The retention times for the designated peaks from the HPLC chromatogram in FIG. 8 are given Table 31. Additional data for representative peaks from the HPLC chromatogram in FIG. 8 are given in Table 32.

TABLE 29
HPLC peak retention times for white ginseng essential oil fraction

| | Ret Time |
|---|---|
| Peak No. | (Min) |
| 4010 | 5.152 |
| 4011 | 5.611 |
| 4012 | 6.272 |
| 4013 | 6.539 |
| 4014 | 7.072 |
| 4015 | 7.224 |
| 4016 | 7.936 |
| 4017 | 8.277 |
| 4018 | 8.875 |
| 4019 | 9.280 |
| 4020 | 10.091 |
| 4021 | 10.421 |
| 4022 | 10.645 |
| 4023 | 10.923 |
| 4024 | 12.021 |
| 4025 | 12.309 |
| 4026 | 12.853 |
| 4027 | 13.931 |
| 4028 | 14.453 |
| 4029 | 15.147 |
| 4030 | 15.456 |
| 4031 | 16.011 |
| 4032 | 16.395 |
| 4033 | 18.208 |
| 4034 | 18.656 |
| 4035 | 19.307 |
| 4036 | 20.768 |
| 4037 | 21.419 |
| 4038 | 22.101 |
| 4039 | 23.520 |
| 4040 | 24.352 |
| 4041 | 25.888 |
| 4042 | 27.339 |
| 4043 | 28.875 |
| 4044 | 30.603 |
| 4045 | 30.660 |
| 4046 | 31.680 |
| 4047 | 32.981 |
| 4048 | 34.155 |
| 4049 | 35.360 |
| 4050 | 37.963 |
| 4051 | 38.773 |
| 4052 | 41.376 |
| 4053 | 42.784 |
| 4054 | 43.275 |
| 4055 | 43.989 |
| 4056 | 46.251 |
| 4057 | 47.488 |
| 4058 | 47.968 |
| 4059 | 48.619 |
| 4060 | 48.875 |
| 4061 | 49.483 |

TABLE 32
HPLC data for red ginseng essential oil fraction

| Peak | | | | | |
|---|---|---|---|---|---|
| Reference | Retention | Area | Height | Width | Theoretical |
| No. | time (Min) | (mAu · min) | (mAu) | (min) | plate |
| 4010 | 5.152 | 270699 | 11709 | 0.53 | 1512 |
| 4013 | 6.539 | 242062 | 9947 | 0.57 | 2106 |
| 4017 | 8.277 | 38650 | 2745 | 0.26 | 16215 |
| 4018 | 8.875 | 73098 | 3339 | 0.45 | 6223 |
| 4019 | 9.280 | 168194 | 6791 | 0.81 | 2100 |
| 4021 | 10.421 | 46687 | 2493 | 0.33 | 15956 |
| 4022 | 10.645 | 18831 | 2287 | 0.14 | 92503 |
| 4024 | 12.021 | 16362 | 1461 | 0.19 | 64046 |
| 4026 | 12.853 | 22373 | 1248 | 0.31 | 27505 |
| 4030 | 15.456 | 41472 | 2329 | 0.35 | 31202 |
| 4032 | 16.395 | 203394 | 6641 | 1.38 | 2258 |
| 4034 | 18.656 | 54002 | 1903 | 0.54 | 19097 |
| 4035 | 19.307 | 1199447 | 53586 | 1.47 | 2760 |
| 4037 | 21.419 | 142247 | 5661 | 0.63 | 18494 |
| 4038 | 22.101 | 696614 | 25600 | 1.42 | 3876 |
| 4039 | 23.520 | 14330 | 574 | 0.52 | 32733 |
| 4040 | 24.352 | 125606 | 3186 | 1.96 | 2470 |
| 4041 | 25.888 | 2339 | 102 | 0.57 | 33004 |
| 4043 | 28.875 | 1692 | 109 | 0.53 | 47491 |
| 4046 | 31.680 | 9819 | 293 | 0.94 | 18173 |
| 4048 | 34.155 | 8113 | 253 | 0.99 | 19044 |
| 4050 | 37.963 | 15537 | 282 | 1.32 | 13234 |
| 4051 | 38.773 | 35999 | 959 | 1.65 | 8835 |
| 4052 | 41.376 | 79054 | 1607 | 2.59 | 4083 |
| 4054 | 43.275 | 49934 | 1479 | 0.69 | 62936 |
| 4055 | 43.989 | 75015 | 1926 | 1.76 | 9995 |
| 4056 | 46.251 | 5586 | 143 | 1.16 | 25436 |

### Example 22

### HPLC Characterization of Ginsenoside Fraction from P. notoginseng

A ginsenoside fraction from P. notoginseng was prepared as described in Example 9. HPLC analysis was carried using the methods and equipment described in Example 17 with the specific conditions described herein. A ginsenoside fraction sample was diluted 1/10 in HPLC-grade methanol to yield a final concentration of about 1 mg/ml. The sample injection volume was 10 μl. The mobile phase components were as follows: mobile phase component A was phosphate buffer, 0.5% phosphoric acid in water, pH 3.5 (“A”); and, mobile phase component B was acetonitrile (“B”). The concentration gradient elution program used was as follows: the mobile phase composition from initial injection through 20 min comprised on a volume basis 79% and 21%, respectively of A and B; and, a linear gradient from 20 to 60 min with the mobile phase changing from 79% to 58% of A and 21% to 42% of B. The total analysis time was for about 60-70 min, the mobile phase flow rate was 1 ml per min and the column temperature was controlled at 40° C. Peak detection was at 203 nm. A typical HPLC chromatogram for an essential oil fraction from P. notoginseng is given in FIG. 9. The retention times for the designated peaks from the HPLC chromatogram in FIG. 9 are given Table 33. Additional data for representative peaks from the HPLC chromatogram in FIG. 9 are given in Table 34.

TABLE 33
HPLC peak retentions times from P. notoginseng
purified ginsenoside fraction

| | Ret Time |
|---|---|
| Peak No. | (Min) |
| 5013 | 8.331 |
| Rg1 | 9.685 |
| Re | 10.720 |
| 5014 | 12.064 |
| 5015 | 20.011 |
| 5016 | 22.699 |
| 5017 | 25.547 |
| 5018 | 32.555 |
| 5021 | 35.712 |
| Rb1 | 37.173 |
| 5022 | 38.517 |
| Rb2 | 42.091 |
| 5023 | 42.539 |
| Rd | 45.205 |
| 5024 | 47.072 |
| 5025 | 50.967 |
| 5026 | 59.093 |
| 5027 | 60.224 |

TABLE 34
HPLC data from P. notoginseng purified ginsenoside fraction

| Peak Name | Retention | | | | |
|---|---|---|---|---|---|
| or Ref. | time | Area | Height | Width | Theoretical |
| Peak No. | (min) | (mAu · min) | (mAu) | (min) | plate |
| GS Re | 10.283 | 5785 | 3037 | 0.03 | 1692 |
| 5015 | 20.011 | 155246 | 2261 | 3.46 | 535 |
| 5016 | 22.699 | 84147 | 3496 | 2.79 | 1059 |
| 5017 | 25.547 | 65412 | 2102 | 1.35 | 5730 |
| 5018 | 32.555 | 308499 | 14232 | 1.05 | 15381 |
| 10 | 35.712 | 1038366 | 26800 | 2.36 | 3664 |
| GS Rb1 | 37.173 | 84690 | 3550 | 1.06 | 19677 |
| GS Rb2 | 42.091 | 210963 | 3385 | 4.46 | 1425 |
| GS Rd | 45.205 | 90094 | 2658 | 1.97 | 8425 |
| 5026 | 59.093 | 460812 | 8152 | 2.15 | 12087 |
| 5027 | 60.224 | 77037 | 2655 | 0.93 | 67095 |

### Example 23

### HPLC Characterization of Ginsenoside Fraction from P. quinquefolius

An affinity adsorbent purified ginsensode fraction from P. quinquefolius was prepared as described in Example 10. HPLC analysis was carried out as described in Example 22. A typical HPLC chromatogram for a purified ginsenoside fraction from P. quinquefolius is given in FIG. 10. The retention times for the designated peaks from the HPLC chromatogram in FIG. 10 are given Table 35. Additional data for representative peaks from the HPLC chromatogram in FIG. 10 are given in Table 36.

TABLE 35
HPLC peak retention time from P. quinquefolius
purified gensenoside fraction

| | Ret Time |
|---|---|
| Peak No. | (Min) |
| Rg1 | 9.984 |
| Re | 10.432 |
| 6010 | 20.128 |
| 6011 | 21.803 |
| 6012 | 26.048 |
| Rb1 | 37.387 |
| Rc | 39.008 |
| Rb2 | 41.963 |
| Rd | 45.685 |
| 6014 | 49.568 |

TABLE 36
HPLC data from P. quinquefolius purified ginsenoside fraction

| Peak Name | Retention | | | | |
|---|---|---|---|---|---|
| or Ref. | time | Area | Height | Width | Theoretical |
| Peak No. | (min) | (mAu · min) | (mAu) | (min) | plate |
| GS Rg1 | 9.984 | 3081 | 0 | 0.06 | 443023 |
| GS Re | 10.432 | 3347571 | 211041 | 0.61 | 4679 |
| 6010 | 20.128 | 657342 | 4914 | 3.65 | 487 |
| 6011 | 21.803 | 43048 | 2621 | 0.69 | 15975 |
| 6012 | 26.048 | 74199 | 2157 | 1.93 | 2914 |
| GS Rb1 | 37.387 | 5729502 | 291369 | 1.51 | 9809 |
| GS Rc | 39.008 | 662060 | 37836 | 1.80 | 7514 |
| GS Rb2 | 41.963 | 405354 | 8222 | 3.27 | 2635 |
| GS Rd | 45.683 | 1217752 | 66322 | 1.45 | 15882 |

### Example 24

### HPLC Characterization of Ginsenoside Fraction from White Ginseng (P. ginseng)

An affinitty adsorbent purified ginsensode fraction from white ginseng (P. ginseng) was prepared as described in Example 11. HPLC analysis was carried out as described in Example 22. A typical HPLC chromatogram for a purified ginsenoside fraction from white ginseng is given in FIG. 11. The retention times for the designated peaks from the HPLC chromatogram in FIG. 11 are given Table 37. Additional data for representative peaks from the HPLC chromatogram in FIG. 11 are given in Table 38.

TABLE 37
HPLC peak retention times from white
ginseng purified ginsenoside fraction

| | Retention Time |
|---|---|
| Peak Reference No. | (Minutes) |
| 7010 | 6.635 |
| 7011 | 7.861 |
| 7012 | 8.683 |
| 7013 | 9.024 |
| Re | 10.272 |
| 7014 | 11.189 |
| 7016 | 12.971 |
| 7018 | 16.875 |
| 7020 | 20.725 |
| 7023 | 30.528 |
| 7024 | 32.821 |
| 7026 | 35.947 |
| Rb1 | 38.549 |
| Rb2 | 40.427 |
| 7029 | 42.773 |
| 7032 | 47.168 |

TABLE 38
HPLC data from white ginseng purified ginsenoside fraction

| Peak Name | Retention | | | | |
|---|---|---|---|---|---|
| or Ref. | time | Area | Height | Width | Theoretical |
| Peak No. | (min) | (mAu · min) | (mAu) | (min) | plate |
| GS Re | 10.272 | 12756 | 2196 | 0.38 | 11691 |
| 7026 | 35.947 | 47194 | 2900 | 0.77 | 34871 |
| GS Rb1 | 38.549 | 1219221 | 71930 | 2.35 | 4305 |
| GS Rb2 | 40.427 | 708862 | 34792 | 1.53 | 11171 |

### Example 25

### HPLC Characterization of Ginsenoside Fraction from Red Ginseng (P. ginseng)

An affinity adsorbent purified ginsensode fraction from red ginseng (P. ginseng) was prepared as described in Example 12. HPLC analysis was carried out as described in Example 22. A typical HPLC chromatogram for a purified ginsenoside fraction from P. quinquefolius is given in FIG. 12. The retention times for the designated peaks from the HPLC chromatogram in FIG. 12 are given Table 39. Additional data for representative peaks from the HPLC chromatogram in FIG. 10 are given in Table 40.

TABLE 39
HPLC peak retention times from red ginseng
purified ginsenoside fraction

| | Retention Time |
|---|---|
| Peak Reference No. | (Minutes) |
| Rg1 | 9.888 |
| 8015 | 11.424 |
| Rf | 29.280 |
| 8016 | 31.563 |
| 8017 | 34.635 |
| 8019 | 36.373 |
| Rb1 | 37.152 |
| Re | 38.987 |
| Rb2 | 41.312 |
| 8022 | 43.904 |
| Rd | 45.675 |
| 8023 | 48.171 |
| 8024 | 57.109 |
| 8025 | 58.720 |

TABLE 40
HPLC data from red ginseng purified ginsenoside fraction

| Peak Name | Retention | | | | |
|---|---|---|---|---|---|
| or Ref. | time | Area | Height | Width | Theoretical |
| Peak No. | (min) | (mAu · min) | (mAu) | (min) | plate |
| GS Rg1 | 9.0888 | 720045 | 6280 | 0.39 | 8690 |
| GS Rf | 29.280 | 285743 | 13808 | 1.46 | 6435 |
| 8016 | 31.563 | 70436 | 3908 | 0.83 | 23138 |
| 8017 | 34.635 | 128707 | 4700 | 1.27 | 11900 |
| 8019 | 36.373 | 134854 | 5517 | 1.08 | 18148 |
| GS Rb1 | 37.152 | 1208866 | 65256 | 1.77 | 7049 |
| GS Rc | 39.424 | 203364 | 12800 | 0.98 | 25893 |
| GS Rb2 | 41.312 | 804060 | 37500 | 1.50 | 12136 |
| GS Rd | 45.675 | 348382 | 18156 | 1.37 | 17784 |
| 8024 | 57.109 | 188668 | 5775 | 1.71 | 17846 |
| 8025 | 60.128 | 82400 | 3249 | 1.97 | 14905 |

### Example 26

### Gas Chromatograph (“GC”) and Mass Spectroscopy (“MS”) Methods

Gas chromatographic analysis of ginseng extracts and fractions was carried using a Hewlett-Packard Model 5890 gas chromatograph. Analysis was carried out using a XTI-5 capillary column (30 m length×0.25 mm ID, Restek) with a film thickness of 0.25 μm and a flow rate of 1 ml/min for the helium carrier gas. The temperature of gasification was 270° C. The column temperature was programmed as follows: 50-140° C. (held for 15 min) at a rate of 10° C./min, then held at 140° C. for 15 min, followed by 140-260° C. at a rate of 15° C./min, and then held at 260° C. The total run time was 52 min and the sample splitting time was 1:50.

Mass spectroscopy analysis was carried using a Hewlett-Packard Model 5899A mass spectrometer. The ion source temperature was 200° C., and the ion source was EI with an ionization energy of 70 eV. The emission current was 300 mA. The data was collected in full scan mode from m/z 40-600 in 1 s cycles.

### Example 27

### GC/MS Characterization of Essential Oil Fraction from P. notoginseng

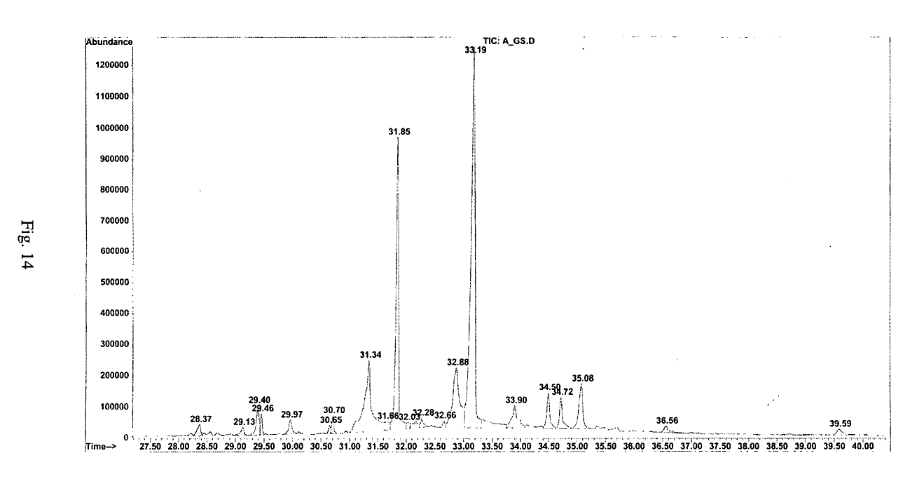

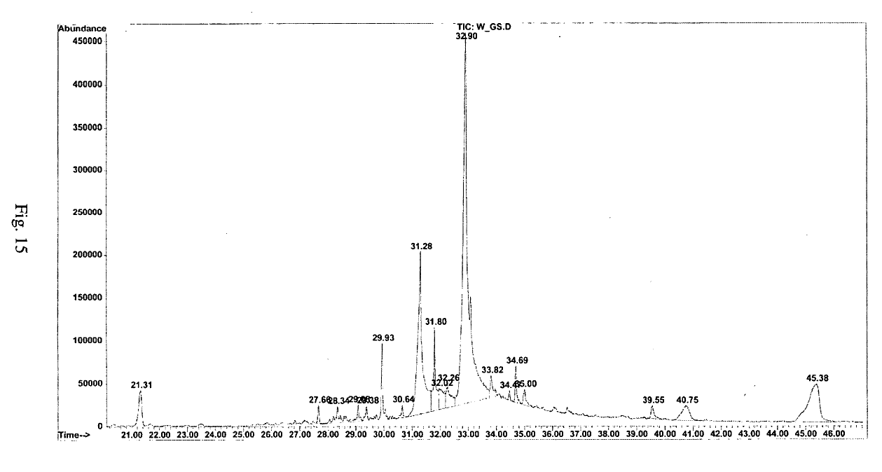

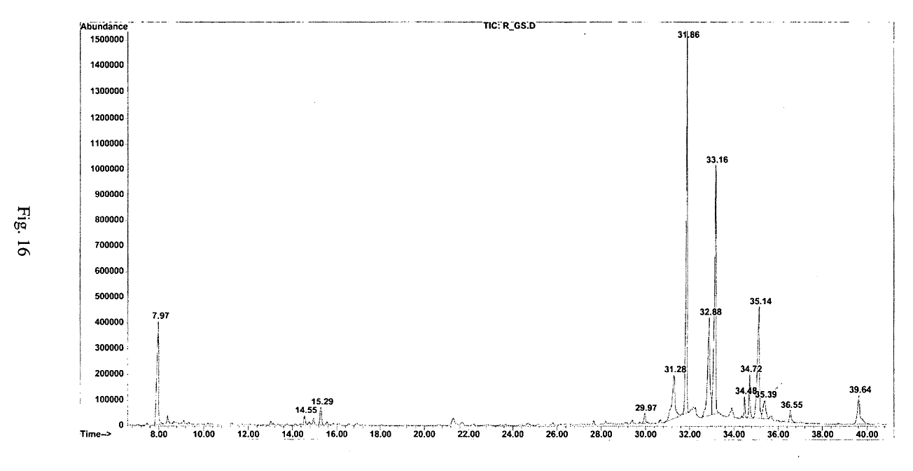

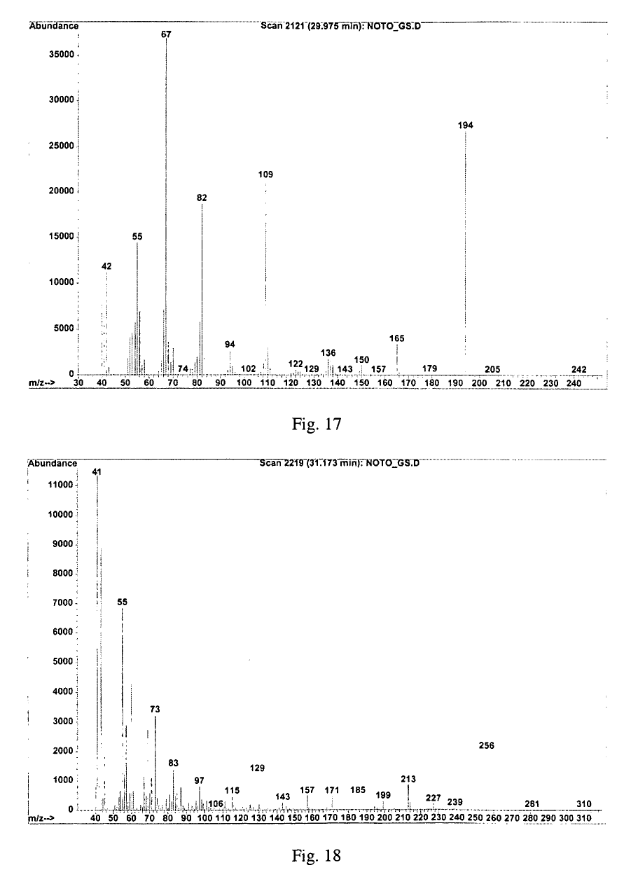

An essential oil fraction from P. notoginseng was prepared as described in Example 1. Gas chromatography analysis was carried out as described in Example 26. An exemplary gas chromatograph of the essential oil fraction from P. notoginseng is shown in FIG. 13. Mass spectral analysis of peaks eluting from the GC was used to help identify the various chemical constituents. Representative mass spectrographs of the essential oil fraction are shown in FIGS. 17, 18, and 19. The MS data (m/z value and abundance) is consistent with the presence the following compounds in the essential oil fraction: (+)-spathulenol (espatulenol), CAS No. 6750-60-3; caffeine, CAS No. 58-08-2; hexadecanoic acid, CAS No. 57-10-3; (−)-caryophyllene oxide, CAS No. 1139-30-6; ethyl heptanoate, CAS No. 106-30-9; trans,trans-octadeca-9,12-dienoic acid methyl ester, CAS No. 2566-97-4; octadec-9-ynoic acid methyl ester, CAS No. 1120-32-7; phenylacetylene, CAS No. 536-74-3; ethylenethiourea, CAS No. 96-45-7; linoleic acid, CAS No. 60-33-3; 4-methyl-pent-2-enoic acid, CAS No. 10321-71-8; 2-methyl-4-nitroimidazole, CAS No. 696-23-1; 9,12-octadecadienal, CAS No. 26537-70-2; mevinphos, CAS No. 7786-34-7; undec-10-ynoic acid, CAS No. 2777-65-3; falcarinol ((Z)-1,9-heptadecadiene-4,6-diyn-3-ol), CAS No. 21852-80-2; and, [1R-(1α,4β,4aα,6β,8aα)]-octahydro-4,8a,9,9-tetramethyl-1,6-methano-1(2H)-naphthol, CAS No. 5986-55-0.

### Example 28

### GC/MS Characterization of Essential Oil Fraction from P. quinquefolius

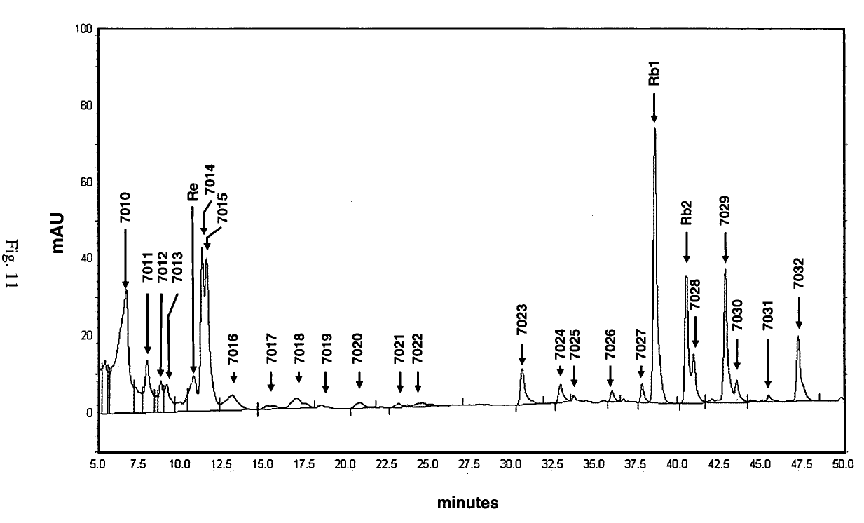

An essential oil fraction from P. quinquefolius was prepared as described in Example 2. Gas chromatography analysis was carried out as described in Example 26. An exemplary gas chromatograph of the essential oil fraction from P. notoginseng is shown in FIG. 14. Mass spectral analysis of peaks eluting from the GC was used to help identify the various chemical constituents. Representative mass spectrographs of the essential oil fraction are shown in FIGS. 20, 21, and 22. The MS data (m/z value and abundance) is consistent with the presence the following compounds in the essential oil fraction: 4,6-diamino-1,3,5-triazin-2(1H)-one, CAS No. 645-92-1; 2,2′-methyliminodiethanol, CAS No. 105-59-9; caffeine, CAS No. 58-08-2; dihydrouracil, 504-07-4; stearic acid (octadecanoic acid), CAS No. 57-11-4; hexadecanoic acid, CAS No. 57-10-3; 4-nitrophenol, CAS No. 100-02-7; linoleic acid, CAS No. 60-33-3; 3-nitrotoluene, CAS No. 99-08-1; 2,3-dihydroxypropyl palmitate, CAS No. 542-44-9; oleic acid, CAS No. 112-80-1; cinnamyl acetate, CAS No. 103-54-8; methyl (9E,12E)-octadeca-9,12-dienoate (methyl linolelaidate), CAS No. 2566-97-4; and, 7-octenoic acid, CAS No. 18719-24-9

### Example 29

### GC/MS Characterization of Essential Oil Fraction from White Ginseng (P. ginseng)

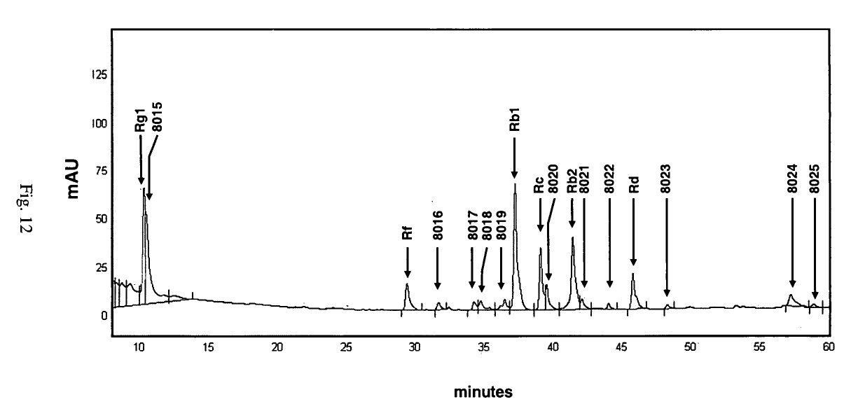

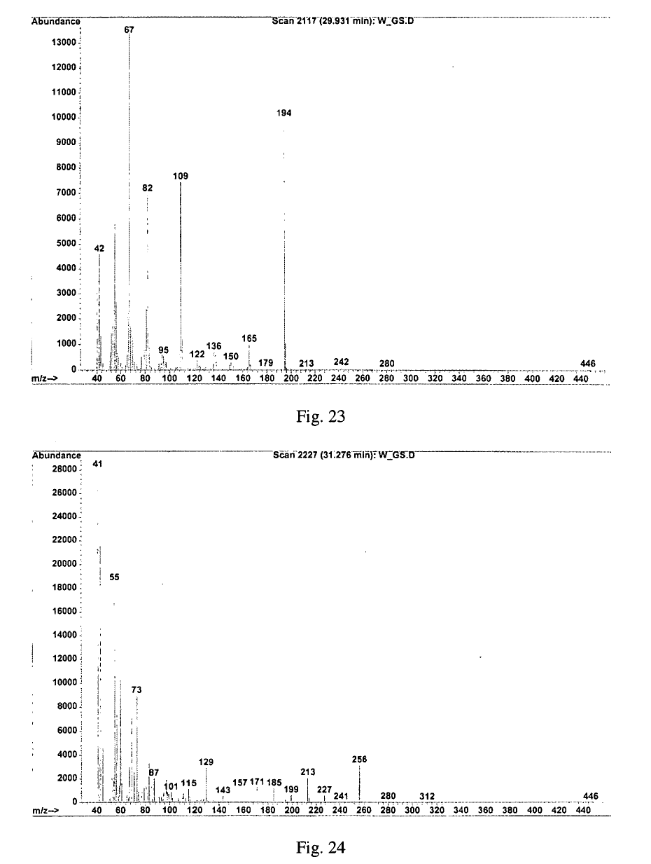

An essential oil fraction from white ginseng (P. ginseng) was prepared as described in Example 3. Gas chromatography analysis was carried out as described in Example 26. An exemplary gas chromatograph of the essential oil fraction from P. notoginseng is shown in FIG. 15. Mass spectral analysis of peaks eluting from the GC was used to help identify the various chemical constituents. Representative mass spectrographs of the essential oil fraction are shown in FIGS. 23, 24, 25, and 26. The MS data (m/z value and abundance) is consistent with the presence the following compounds in the essential oil fraction: (−)-spathulenol, CAS No. 77171-55-2; (+)-spathulenol, CAS No. 6750-60-3; (−)-caryophyllene oxide, CAS No. 1139-30-6; 1-methyl-5-nitro-1H-imidazole, CAS No. 3034-42-2; 2-ethyl-2-methyloxirane, CAS No. 30095-63-7; caffeine, CAS No. 58-08-2; dihydrouracil, CAS No. 504-07-4; hexadecanoic acid, CAS No. 57-10-3; methyl (9E,12E)-octadeca-9,12-dienoate (methyl linolelaidate), CAS No. 2566-97-4; linoleic acid, CAS No. 60-33-3; undec-10-ynoic acid, CAS No. 2777-65-3; phenylacetylene, CAS No. 536-74-3; sinalbin, CAS No. 27299-07-6; stigmasta-5,22-dien-3-β-ol, CAS No. 83-48-7; (3β,24S) -stigmast-5-en-3-ol, CAS No. 83-47-6; stigmast-5-en-3-β-ol, CAS No. 83-46-5; (3β,24ξ)-stigmast-5-en-3-ol, CAS No. 19044-06-5; 4-methyl-1,4-heptadiene, CAS No. 13857-55-1; 9,12-octadecadienal, CAS No. 26537-70-2; 7,8-epoxyoctene, CAS No. 19600-63-6; 4-nonyne, CAS No. 20184-91-2; 2-cyclopenten-1-undecanoic acid, CAS No. 459-67-6; falcarinol ((Z)-1,9-heptadecadiene-4,6-diyn-3-ol), CAS No. 21852-80-2; and, N-methylcaprolactam, CAS No. 2556-73-2.

### Example 30

### GC/MS Characterization of Essential Oil Fraction from Red Ginseng (P. ginseng)

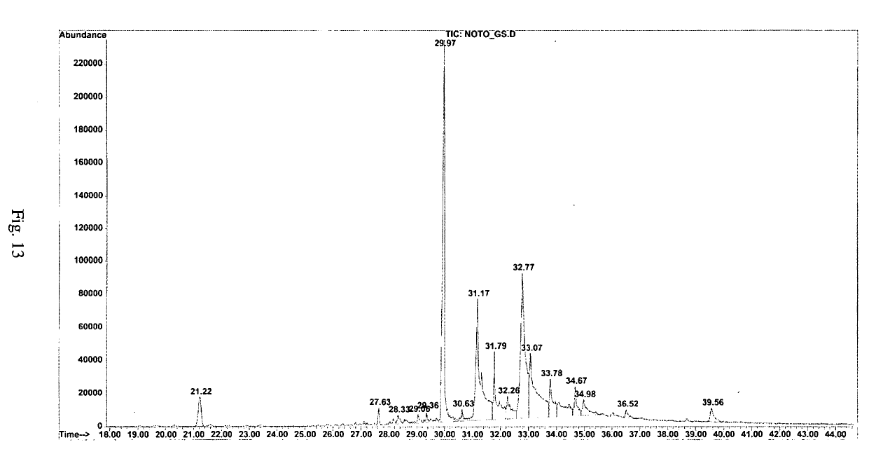

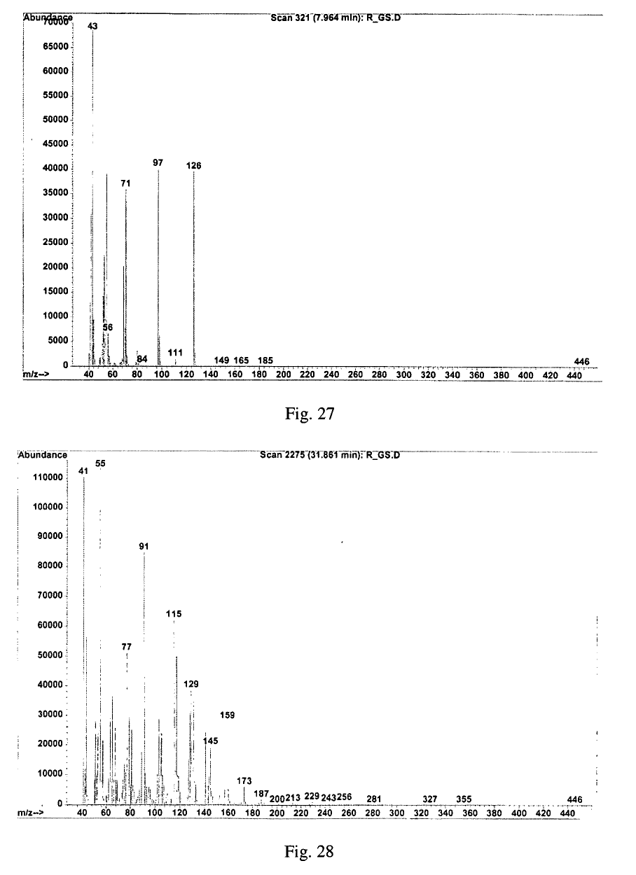

An essential oil fraction from red ginseng (P. ginseng) was prepared as described in Example 3. Gas chromatography analysis was carried out as described in Example 26. An exemplary gas chromatograph of the essential oil fraction from P. notoginseng is shown in FIG. 16. Mass spectral analysis of peaks eluting from the GC was used to help identify the various chemical constituents. Representative mass spectrographs of the essential oil fraction are shown in FIGS. 27, 28, 29, 30, and 31. The MS data (m/z value and abundance) is consistent with the presence the following compounds in the essential oil fraction: 3-hydroxy-2-methyl-4-pyrone, CAS No. 118-71-8; pyrogallol, CAS No. 87-66-1; [1aR-(1aα,7α,7aα,7bα)]-1a,2,3,5,6,7,7a,7b-octahydro-1,1,7,7a-tetramethyl-1H-cyclopropa[a]naphthalene, CAS No. 17334-55-3; [1aR-(1aα,4aα,7α,7aβ,7bα)]-decahydro-1,1,7-trimethyl-4-methylene-1H-cycloprop[e]azulene, CAS No. 489-39-4; caryophyllene, CAS No. 87-44-5; 1R-(1R*,4Z,9S*)]-4,11,11-trimethyl-8-methylenebicyclo[7.2.0]undec-4-ene, CAS No. 118-65-0; caffeine, CAS No. 58-08-2; hexadecanoic acid, CAS No. 57-10-3; 4-methyl-2-phenyl-2-pentenal, CAS No. 26643-91-4; (Z)-9,17-Octadecadienal, CAS No. 56554-35-9; linoleic acid, CAS No. 60-33-3; ethylidenecycloheptane, CAS No. 10494-87-8; Octa-1,7-diyne, CAS No. 871-84-1; 3-(phenylmethyl)sydnone, CAS No. 16844-42-1; phenylacetylene, 536-74-3; diisopropyl adipate, CAS No. 6938-94-9; 2,3-dihydroxypropyl palmitate, CAS No. 542-44-9; 9Z,12Z-octadecadienoic acid (2-linoleoyl glycerol), CAS No. 3443-82-1; and, 3-ethenyl-cyclooctene, CAS No. 2213-60-7.

### Example 31

### Determination of Polysaccharide Concentration in the Panax Species Purified Polysaccharide Fraction

All of the dried polysaccharide extracts from P. notoginseng (Example 13), P. quinquefolius (Example 14), white ginseng (Example 15), and red ginseng (16) were colorless indicating that the tannins and other polyphenolic chemical constiuents were extracted with ethanol during the extraction of the ginsenosides (Step 2). When various amounts of salts (up to 100 mg/2 gm (solution dry mass weight) as well as citric acid and acetic acid were added to the polysaccharide extract solutions, no precipitate was observed indicating that the protein content of the polysaccharide extract fractions were low. Furthermore, dissolving the freezed dry polysaccharide extract fractions in 10 volumes of ethanol revealed that less than 1% of the polysaccharide extract fractions were soluble in ethanol. Therefore, these Panax species olysaccharide extract fractions were highly purified, probably greater than 99%, mixtures of polysaccharides of various molecular weights.

More directly, the amount of carbohydrate in all of the purified polysaccharide fractions were determined using the anthrone colorimetric method. Typically, about 0.18 gram of anthrone (CAS No. 90-44-8, obtained from Sigma-Aldrich), which is also called 9,10-dihydro-9-oxoanthracene, was mixed with about 50 ml of concentrated sulfuric acid, (95.7%, obtained from Fisher). The solution of anthrone and sulfuric acid was shaken and then immersed in an ice water bath. Calibration of the spectrophotometer (Thermo Spectronic 20D+) was accomplished using lactose (CAS No. 63-42-3, obtained from Acros) as a standard. A lactose standard solution (0.05% wt/vol) was prepared by dissolving about 15 mg of lactose in about 30 ml of distilled water. Specific volumes of the lactose standard solutions (typically about 0, 200, 400, 600, and 800 μl) were pipetted into test tubes, and the volume was adjusted to a final volume of 1 ml using distilled water. Anthrone solution (2 ml), prepared as described above, was gradually added to each 1 ml lactose standard samples while the test tube was vigorously shaken. Following mixing, the anthrone—sample solutions were then stored in an ice bath for 30 minutes, followed by warming to room temperature. The absorbance of each sample was determined at 625 nm using distilled water as a blank. The absorbance was plotted versus the concentration of lactose to obtain a standard curve. Samples of the purified polysaccharide fractions (about 10 μl) were diluted to 1 ml with distilled water. To the diluted polysaccharide samples was gradually added about 2 ml of the anthrone reagent with vigorous shaking. Following mixing, the anthrone—polysaccharide solutions were stored for 30 minutes in an ice bath, and then warmed to room temperature. The absorbance at 625 nm was determined for each anthrone—polysaccharide sample solutions. The amount of carbohydrate in each sample was determined using the lactose standard curve prepared as described above. Typically, using this method, it was determined that all of the polysaccharides fraction derived from all of the Panax species studied contained greater than 90% by weight carbohydrate components.

### Example 32

### P. notoginseng Extract Dosage Form Composition

An extract of P. notoginseng was prepared according to the present invention and used to prepare a dosage form composition suitable for tablets, capsules, or powder for addition to water or other solution as a drinkable solution. The dosage form composition was prepared according to the formulation given in Table 41, wherein the amounts given are the amounts per single dosage form.

TABLE 41
P. notoginseng formulation composition

| | | |
|---|---|---|
| Extract of P. notoginseng | 150.0 | mg |
| Essential Oil (2 mg, 1.3% dry weight) | | |
| Total Ginsenosides (38 mg, 25.3% dry weight) | | |
| Polysaccharides (110 mg, 73.3% dry weight) | | |
| Stevioside (Extract of Stevia) | 12.5 | mg |
| Carboxymethylcellulose | 35.5 | mg |
| Lactose | 77.0 | mg |
| TOTAL | 275.0 | mg |

The novel extract of P. notoginseng comprises an essential oil, ginsenosides, and polysaccharides by percentage mass weight greater than that found in the natural rhizome material or convention extraction products. The formulations can be made into any oral dosage form and administered daily or to 15 times per day as needed for the physiological, psychological and medical effects desired (enhanced memory and cognition, relief from chronic fatigue syndrome, enhancement of male erectile function) and medical effects (anti-oxidation, anti-platelet aggregation, cardiovascular and cerebrovascular disease prevention and treatment, anti-hypercholesterolemia, cytoprotection, nervous system protection, neurological degenerative disease such as Alzheimer's and Parkinson's disease prevention and treatment, anti-inflammatory, immune enhancement, anti-viral, pulmonary disease, hepatic protection and diseases, hypoglycemic and anti-diabetes, and cancer prophylaxis and treatment). The dosage composition as provided in Table 41 may be compressed into a tablet, used in a gelcap, or used in a fast-dissolve table.

### Example 33

### P. quinquefolius Extract Dosage Form Composition

An extract of P. quinquefolius was prepared according to the present invention and used to prepare a dosage form composition suitable for tablets, capsules, or powder for addition to water or other solution as a drinkable solution. The dosage form composition was prepared according to the formulation given in Table 42, wherein the amounts given are the amounts per single dosage form.

TABLE 42
P. quinquefolius formulation composition

| | | |
|---|---|---|
| Extract of P. quinquefolius | 150.0 | mg |
| Essential Oil (2.0 mg, 1.3% dry weight) | | |
| Total Ginsenosides (20.0 mg, 13.3% dry weight) | | |
| Polysaccharides (128.0 mg, 85.4% dry weight) | | |
| Vitamin C | 15.0 | mg |
| Sucralose | 35.0 | mg |
| Mung bean powder (10:1) | 50.0 | mg |
| Mocha flavor | 40.0 | mg |
| Flavor (Chocolate, strawberry, mocha, etc.) | 10.0 | mg |
| TOTAL | 300.0 | mg |

Mung Bean Powder 10:1 refers to water content (10 parts Mung Bean to 1 part water). It is used as a binder.

The novel extract composition of P. quinquefolius comprises an essential oil, ginsenosides, and polysaccharide chemical constituents by % mass weight greater than that found in the natural plant material or conventional extraction products. The formulation can be made into any oral dosage form and administered safely up to 15 times per day as needed for the physiological, psychological and medical effects desired (see Example 1, above). The dosage composition as provided in Table 42 may be compressed into a tablet, used in a gelcap, or used in a fast-dissolve table.

### Ezample 34

### White Ginseng (P. ginseng) Extract Dosage Form Composition

An extract of white ginseng (P. ginseng) was prepared according to the present invention and used to prepare a dosage form composition suitable for tablets, capsules, or powder for addition to water or other solution as a drinkable solution. The dosage form composition was prepared according to the formulation given in Table 43, wherein the amounts given are the amounts per single dosage form. Table 43.

TABLE 43
White ginseng formulation composition

| | | |
|---|---|---|
| Extract of White Ginseng (P. ginseng) | 150.0 | mg |
| Essential Oil (5.0 mg, 3.3% dry weight) | | |
| Total Ginsenosides (45.0 mg, 30.0% dry weight) | | |
| Polysaccharides (100.0 mg, 66.7% dry weight) | | |
| Vitamin C | 15.0 | mg |
| Sucralose | 35.0 | mg |
| Mung bean powder (10:1) | 30.0 | mg |
| Flavor (Strawberry) | 60.0 | mg |
| X-base M-500 | 54.0 | mg |
| X-base Xanthan gum | 1.0 | mg |
| TOTAL | 350.0 | mg |

The novel extract composition of White Ginseng (P. ginseng) comprises an essential oil, ginsenosides, and polysaccharides chemical constituents by % mass weight greater than that found in the natural plant material or conventional extraction products. Note also the profile change in the White Ginseng extract composition (the essential oil/ginsenosides ratio in feedstock was 1/6.4 and in extract composition is 1/5; the essential oil/polysaccharides ratio in feedstock was 1/35 and in the extract composition is 1/20; and the ginsenosides/polysaccharides ratios was 1/5.4 and the extract composition is 1/4). The formulation can be made into any oral dosage for and administered safely up to 15 times per day as needed for the physiological, psychological, and medical effects desired (see Example 1, above). The dosage composition as provided in Table 43 may be compressed into a tablet, used in a gelcap, or used in a fast-dissolve table.

Whereas this invention has been described in detail with particular reference to specific embodiments, it will be apparent to those skilled in the art that various modifications and variations can be made in the present invention in light of the above teachings without departing from the scope or spirit of the invention. Other embodiments of the invention will be apparent to those skilled in the art from consideration of the specification and practice of the invention disclosed herein. It is intended that the specification and examples be considered exemplary only, with a true scope and spirit of the invention being indicated by the following claims.

### REFERENCES

U.S. Pat. Nos. 3,464,972; 3,883,425; 3,886,272; 3,901,875; 4,157,894; 5,776,460; 6,432,454;
1. Sticher O. Chemtech 28(4):26, 1998.
2. Tang G and Eisenbrand G. Chinese Drugs of Plant Origin, Springer-Verlag: New York, 1984 Vol. 46:1.
3. Shibata S et al. In: Economic and Medicinal Plant Research; Wagner H et al (eds); Academic Press: London, 1985; Vol. 1:217.
4. Zhang S et al. Planta Med 56:298, 1990.
5. Liu C-X and Xiao P-G. J Ethnopharmacol 36:27, 1992.
6. Sonnenborn U. Dtsch Apoth Ztg 127:433, 1987.
7. Sonnenborn U. Proppert, Y Z Phytother 11:35, 1990.
8. Soldati F and Sticher O. Planta Med 39:348, 1980.
9. Angell M and Kassirer J P. N Engl J Med 339:839, 1998.
10. Hershey M R et al. Am J Clin Nutr 73(6):1101, 2001.
11. Deng H W et al. Biochem Arch 6:359, 1990.
12. Deng H and Zang J. Chin Med J 104:395, 1991.
13. Lee S J et al. Cancer Lett 144(1):39, 1999.
14. Maffei F et al. Planta Med 65(7):614, 1999.
15. Voces J et al. Comp Biochem Physiol C Pharmacol Toxicol Endocrinol 123(2):175, 1999.
16. Chang T O. Arch Intern Med 160:3329, 2000.
17. Lee J Y et al. Life Sci 75f(13):1621, 2004
18. Zhen S Y et al. Zhougguo Zhong Xi Yi Jie He Za Zhi. 24(6):541, 2004.
19. Shao ZH et al. Biochim Biophys Acta. 1670(3):165, 2004.
20. Sui D Y et al. Zhongguo Zhong Yao Za Zhi. 26(6):416, 2001.
21. Caron M F et al. Ann Pharmacother. 36(5):758, 2002
22. Gao Q et al. Planta Med 55:9, 1989.
23. Yamada H and Kiyhara H. Abstr Chin Med 3:104, 1989.
24. Tomoda M et al. Biol Pharm Bull 16:22, 1993.
25. Tomoda M et al. Biol Pharm Bull 17:1287, 1994.
26. Tomoda Metal. Biol Pharm Bull 16:1087, 1993.
27. Liu J et al. Ageing Dev 83:43, 1995.
28. Yamada H et al. Phytother Res 9:264, 1995.
29. Wang M et al. J Pharm Pharmacol. 53(11):1515, 2001.
30. Yoshikawa M et al. Chem Pharm Bull (Tokyo). 49(11):1452, 2001.
31. Shin J Y et al. Immunopharmacol Immunotoxicol. 24(3):469, 2002.
32. Rivera E et al. Vaccine 21(11-12):1149, 2003.
33. Sun H X et al. Acta Pharmacol Sin. 24(11):1150, 2003.
34. McElhaney J E et al. J Am Geriatr Soc. 52(1):13, 2004.
35. Sun X-B et al. J Ethnopharmacol 31:101, 1991.
36. Wen T-C et al. Acta Neuropath 91:15, 1996.
37. Rudakewich M et al. Planta Med. 67(6):533, 2001.
38. Lee T F et al. Planta Med. 67(7):634, 2001.
39. Liao B et al. Exp Neurol. 173(2):224, 2002.
40. Van Kampen J et al. Exp Neurol. 184(1):521, 2003.
41. Kimura Y et al. J Pharm Pharmacol 40:838, 1988.
42. Teng C-M et al. Biochem Biophys Acta 990:315, 1989.
43. Kuo S-C et al. Planta Med 56:164, 1990.
44. Liu T et al. Zhongguo Zhong Yao Za Zhi. 27(8):609, 2002.
45. Nah S-Y et al. Proc Natl Acad Sci 92:8739, 1995.
46. Ahn B-Z and Kim S-I. Arch Pharm 321:61, 1988.
47. Matsunaga H et al. Chem Pharm Bull 37:1279, 1989.
48. Matsunaga H et al. Chem Pharm Bull 38:3480, 1990.
49. Matsunaga H et al. Cancer Chemother Pharmacol 35:291, 1995.
50. Mochizuki M et al. Biol Pharm Bull 18:1197, 1995.
51. Fei X F et al. Acta Pharmacol Sin. 23(4):315, 2002.
52. Yun T K et al. J Korean Med Sci. 16:S6, 2001.
53. Ro J Y et al. Int J Immunopharmacol. 20(11):625, 1998.
54. Cabral de Oliveira A C et al. Comp Biochem Physiol C Toxicol Pharmacol. 30(3):369, 2001.
55. Inoue M et al. Phytomed 6(4):257, 1999.
56. Kim S H and Park K S. Pharmacol Res. 48(5):511, 2003.
57. Sotaniemi E et al. Diabete Care 18(10):1373, 1995.
58. Dey L et al. Phytomedicine 10(6-7):2003.
59. Bae J W and Lee M H. J Ethnopharmacol 91(1):137, 2004.
60. Xie J T et al. Phytomedicine 11(2-3):182, 2004.
61. Gross D et al. Monaldi Arch Chest Dis. 57(5-6):242, 2002.
62. Lin C F et al. Phytother Res. 17(9):1119, 2003.
63. Park E J et al. Basic Clin Pharmacol Toxicol. 94(3):298, 2004.
64. Choi H et al. Int J Impot Res. 7(3):935,1991.
65. Choi H et al. J Urol. 162(4):1508, 1999.
66. Murphy L L and Lee T J. Ann NY Acad Sci. 962:372, 2002.
67. Hong B et al. J Urol. 168(5):2070, 2002.
68. Yamazaki M et al. Biol Pharm Bull. 24(2):1434, 2001.
69. Scholey A B and Kennedy D O. Hum Psychopharmacol. 17(1):35, 2002.
70. Hartz A J et al. Psychol Med. 34(1):51, 2004.
71 Fulder S J et al. Proc 4^{th} Int Ginseng Symposium 4:215-223, 1984.
72. Yun Y S et al. Plant Med 59:521, 1993.
73. Kim K-H et al. Planta Med 64:110, 1998.
74. Lee Y-S et al. Anticancer Res 17:323, 1997.

What is claimed is:

1. A method for extracting a Panax species to produce a Panax extract composition comprising, sequentially extracting a Panax species plant material to yield an essential oil fraction, a ginsenoside fraction and a polysaccharide fraction, wherein the essential oil fraction is derived by extracting plant feedstock material by supercritical carbon dioxide extraction, the ginsenoside fraction is extracted from the remainder of the essential oil extraction by hydroalcoholic extraction, and the polysaccharide fraction is derived by hot water extraction of the remainder of the ginsenoside extraction.

2. The method of claim 1, wherein ginsenoside extraction comprises,
a) contacting a remainder of a feedstock material from an extraction of an essential oil extraction by supercritical carbon dioxide with a hydroalcoholic mixture for a time sufficient to extract ginsenosides;
b) passing an aqueous solution of extracted ginsenosides from the hydroalcoholic mixture through an adsorbent resin column wherein the ginsenosides are adsorbed; and
c) eluting the ginsenosides from adsorbent resin.

3. A Panax species extract composition, comprising, an essential oil fraction composition greater than 0.5% by mass weight of the total weight, a ginsenoside fraction composition and a polysaccharide fraction composition.

4. The Panax extract composition of claim 3, wherein the essential oil composition comprises the fractionation profile FIG. 5 for P. notoginseng or FIG. 6 for P. quinquifolius or FIG. 7 for P. ginseng (white ginseng) or FIG. 8 for P. ginseng (red ginseng).

5. The Panax extract composition of claim 3, wherein the ginsenoside composition comprises the fractionation profile of FIG. 9 for P. notoginseng or FIG. 10 for P. quinquefolius or FIG. 11 for P. ginseng (white ginseng) or FIG. 12 for P. ginseng (red ginseng).

6. The Panax extract composition of claim 3, wherein the essential oil composition and the ginsenoside composition comprises the fractionation profiles of FIG. 5 and FIG. 9 for P. notoginseng, FIG. 6 and FIG. 10 for P. quinquefolius, FIG. 7 and FIG. 11 for P. ginseng (white ginseng), and FIG. 8 and FIG. 12 for P.ginseng (red ginseng), respectively.

7. The Panax extract composition of claim 3, wherein the essential oil composition comprises (+)-spathulenol (spathulenol, espatulenol); caffeine; hexadecanoic acid; (−)-caryophyllene oxide; ethyl heptanoate; trans,trans-octadeca-9,12-dienoic acid methyl ester; octadec-9-ynoic acid methyl ester; phenylacetylene; ethylenethiourea; linoleic acid; 4-methyl-pent-2-enoic acid; 2-methyl-4-nitroimidazole; 9,12-octadecadienal; mevinphos; undec-10-ynoic acid; falcarinol ((Z)-1,9-heptadecadiene-4,6-diyn-3-ol); [1R-(1α,4β,4aα,6β,8aα)]-octahydro-4,8a,9,9tetramethyl-1,6-methano-1(2H)-naphthol; 4,6-diamino-1,3,5-triazin-2(1H)-one; 2,2′-methyliminodiethanol; dihydrouracil; stearic acid (octadecanoic acid); 4-nitrophenol; 3-nitrotoluene,; 2,3-dihydroxypropyl palmitate; oleic acid; cinnamyl acetate; 7-octenoic acid; (−)-spathulenol,; 1-methyl-5-nitro-1H-imidazole; 2-ethyl-2-methyloxirane; methyl (9E,12E)-octadeca-9,12-dienoate; sinalbin, stigmasta-5,22-dien-3-β-ol; (3β,24S)-stigmast-5-en-3-ol; stigmast-5-en-3-β-ol; (3β,24ξ)-stigmast-5-en-3-ol; 4-methyl-1,4-heptadiene; 9,12-octadecadienal; 7,8-epoxyoctene, 4-nonyne; 2-cyclopenten-1-undecanoic acid; 3-hydroxy-2-methyl-4-pyrone; pyrogallol; [1aR-(1aα,7α,7aα,7bα)]-1a,2,3,5,6,7,7a,7b-octahydro-1,1,7,7a-tetramethyl-1H-cyclopropa[a]naphthalene; [1aR-(1aα,4aα,7α,7aβ,7bα)]-decahydro-1,1,7-trimethyl-4-methylene-1H-cycloprop[e]azulene; caryophyllene; 1R-(1R*,4Z,9S*)]-4,11,11-trimethyl-8-methylenebicyclo[7.2.0]undec-4-ene; 4-methyl-2-phenyl-2-pentenal; (Z)-9,17-Octadecadienal; ethylidenecycloheptane, 8; octa-1,7-diyne; 3-(phenylmethyl)sydnone; diisopropyl adipate; 2,3-dihydroxypropyl palmitate; 9Z,12Z-octadecadienoic acid (2-linoleoyl glycerol); and, 3-ethenyl-cyclooctene.

8. The Panax extract composition of claim 3, wherein the composition comprises greater than 0.5% wt essential oil, greater than 12.0% wt ginsenoside, and greater than 50% polysaccharide.

9. The Panax extract composition of claim 8, wherein the essential oil composition comprises the fractionation profile FIG. 5 for P. notoginseng or FIG. 6 for P. quinquifolius or FIG. 7 for P. ginseng (white ginseng) or FIG. 8 for P. ginseng (red ginseng).

10. The Panax extract composition of claim 8, wherein the ginsenoside composition comprises the fractionation profile of FIG. 9 for P. notoginseng or FIG. 10 for P. quinquefolius or FIG. 11 for P. ginseng (white ginseng) or FIG. 12 for P. ginseng (red ginseng).

11. The Panax extract composition of claim 8, wherein the essential oil composition and the ginsenoside composition comprises the fractionation profiles of FIG. 5 and FIG. 9 for P. notoginseng, FIG. 6 and FIG. 10 for P. quinquefolius, FIG. 7 and FIG. 11 for P. ginseng (white ginseng), and FIG. 8 and FIG. 12 for P.ginseng (red ginseng), respectively.

12. The Panax species extract composition of claim 3, wherein the composition comprises a ginsenoside composition or a polysaccharide composition in a percent weight concentration that is more or less than the percent weight concentration found in native Panax species plant material.

13. The composition of claim 12, wherein the ginsenoside composition is in a percent weight concentration that is less than or more than the percent weight concentration found in native Panax species plant material.

14. The composition of claim 12, wherein the ginsenoside composition and the polysaccharide concentraction is in a percent weight concentration that is more than the percent weight concentration found in native Panax species plant material.

15. The composition of claim 12, wherein the polysaccharide composition is in a percent weight concentration that is less than or more than the percent weight concentration found in native Panax species plant material.

16. The composition of claim 12, wherein the essential oil composition is from 0.001 to 0.5 or from 1.5 to 200 times the percent weight concentration of native Panax species plant material.

17. The composition of claim 12, wherein the ginsenoside composition is from 0.01 to 0.5 or from 1.5 to 100 times the percent weight concentration of native Panax species plant material.

18. The composition of claim 12, wherein the polysaccharide composition is from 0.1 to 0.5 or from 1.5 to 6 times the percent weight concentration of native Panax species plant material.

19. A method for treating and preventing disease in humans, comprising, administering oral delivery compositions to humans comprising effective amounts of compositions of extracts of Panax species comprising fractions of essential oils, ginsenosides and polysaccharides, for anti-oxidant activity, cardiovascular disease prevention and treatment, cerebrovascular disease prevention and treatment, neurological protection, anti-dementia, anti-neurological degenerative disease, Alzheimerís, Parkinsonís disease, anti-hypercholesterolemia, anti-platelet aggregation, immune enhancement, anti-viral, hypoglycemic, diabetes therapy, pulmonary disease prevention, hepatic disease, anti-inflammatory conditions, enhancement of male erectile capacity, enhancement of memory and cognition, and treatment of chronic fatigue syndromes.

20. The method of claim 19, wherein the composition comprises an essential oil fraction composition greater than 0.5% by mass weight of the total weight, a ginsenoside fraction composition and a polysaccharide fraction composition.
